(19) United States
(12) Patent Application Publication
(10) Pub. No.: US 2024/0327457 A1
(43) Pub. Date: Oct. 3, 2024

(54) LIPOPEPTIDES, NANOPARTICLES AND METHODS OF USE

(71) Applicant: Wisconsin Alumni Research Foundation, Madison, WI (US)

(72) Inventors: Shaoqin Gong, Middleton, WI (US); Min Zhu, Madison, WI (US); Xianghui Xu, Changsha (CN)

(73) Assignee: Wisconsin Alumni Research Foundation, Madison, WI (US)

(21) Appl. No.: 18/434,662

(22) Filed: Feb. 6, 2024

Related U.S. Application Data
(60) Provisional application No. 63/443,675, filed on Feb. 6, 2023.

(51) Int. Cl. C07K 5/02; A61K 9/51; A61K 31/7105; A61K 31/711; C07K 1/107

(52) U.S. Cl. CPC C07K 5/0215; A61K 9/5123; A61K 31/7105; A61K 31/711; C07K 1/1077

(57) ABSTRACT

The present technology provides disulfide-containing lipopeptides of Formula I. Such lipids may be incorporated into lipid nanoparticles for delivery of nucleic acids, nucleotides (e.g., NAD⁺ or NADH), polypeptides (including proteins), and complexes of proteins and nucleic acids, e.g., Cas9 RNP. The present technology also provides methods for delivering the nanoparticles to a cell and methods of treating a condition or disorder using the lipid nanoparticles.

### CROSS-REFERENCE TO RELATED APPLICATION

The present application claims benefit of U.S. Provisional Patent Application No. 63/443,675, filed on Feb. 6, 2023, which is hereby incorporated by reference in its entirety.

### FIELD

The present technology relates generally to lipopeptides and disulfide-containing lipopeptides. The lipopeptides are ionizable and may be incorporated into lipid nanoparticles (e.g., liposome or solid nanoparticles) suitable for the delivery of nucleotides (e.g., NAD⁺, NADH) nucleic acids, proteins, mixtures of nucleic acids (e.g., mRNA and sgRNA, or mRNA and crRNA), or mixtures of protein and nucleic acid(s) (e.g., protein and sgRNA, protein and crRNA) to cells. Methods of preparing and using the lipid nanoparticles are also provided.

### BACKGROUND

Safe and efficient delivery of biologics including nucleic acids, proteins, gene-editing systems (e.g., CRISPR genome editors), epigenetic-editing systems and other biomacromolecules to target cells for therapeutic purposes remains a challenge. Nucleic acids, including DNA, mRNA, nucleotides (e.g., NAD⁺ or NADH), antisense oligonucleotide (ASO), and small interfering RNA (siRNA) are widely used for gene therapy. CRISPR genome editing is a powerful technique capable of gene deletion, insertion, correction and other alterations; thus CRISPR genome editing can potentially be used to treat the root causes of many diseases. CRISPR genome editors can be delivered in various forms including pDNA encoding CasX (e.g., X=9, 12, 13, 14, Φ) protein and gRNA (e.g., sgRNA, crRNA, or tracrRNA+crRNA), CasX mRNA/gRNA, CasX protein/gRNA, ribonucleoproteins (RNPs), and those in combination of single-stranded oligonucleotide DNA (ssODN) (e.g., RNP+ssODN for gene correction). Other genome editors also include base editors, prime editors, and more. Epigenetic editing (e.g., using deactivated CasX proteins (e.g., dCas9)) and mitochondrial gene editing can also lead to new gene therapy. Under physiological conditions, naked biologics (such as nucleic acids, proteins, and genome editors) are prone to enzymatic degradation. Moreover, the delivery efficiency is negligible due to the lack of cellular uptake and endosomal escape capability. In addition, efficient delivery of CRISPR genome editors, such as RNP+ssODN, is hindered by its heterogenous charges and complicated structures. To address such issues, non-viral nanovectors have been investigated for the delivery of biomacromolecules. Nonetheless, current state-of-the-art non-viral nanovectors often suffer from low payload encapsulation content, low efficiency and high cytotoxicity among other issues.

### SUMMARY OF THE INVENTION

As disclosed herein, the present technology provides new lipopeptides, including disulfide-containing lipopeptides that can form lipid nanoparticles (LNP) such as lipoplexes or solid nanoparticles which safely and efficiently deliver biomolecules into cells, particularly animal cells, e.g., mammalian cells. In various aspects and embodiments the present LNP technology provides one or more: (1) high loading content and loading efficiency, while maintaining the payload activity, (2) versatile surface chemistry (e.g., ligand conjugation) to facilitate the payload delivery to target cells, (3) excellent biocompatibility, (4) efficient endo/lysosomal escape capability, and (5) rapid payload release in the target cells, leading to high editing efficiency and good biocompatibility. They are also easy to handle, store and transport.

The present technology provides a disulfide-containing lipopeptide that may be used to form nanoparticles such as lipoplexes or solid LNPs, or coat other nanoparticles such as silica nanoparticles (SNP). Thus, in one aspect the present technology provides a compound of Formula I,

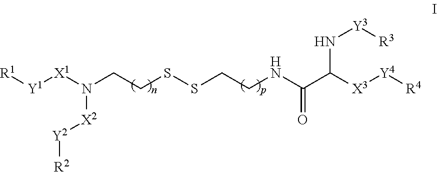

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y², are each independently absent, C(O)O, or C(O)NH;
Y³ is absent, C(O), C(O)O, or C(O)NH;
Y⁴ is C(O)O, C(O)NH, NHC(O)O, or NHC(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid residue, peptide, or isopeptide; and further provided that if one of R³ and R⁴ is absent, the other is present; and
n and p are each independently 1, 2, 3, 4, or 5.

In one embodiment, the present technology provides a compound of Formula I that is a compound of Formula IA,

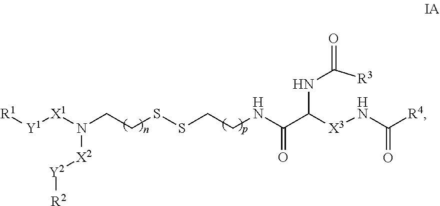

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
n and p are each independently 1, 2, 3, 4, or 5.

In any embodiments, the compound of Formula I (or IA) is a compound of Formula II,

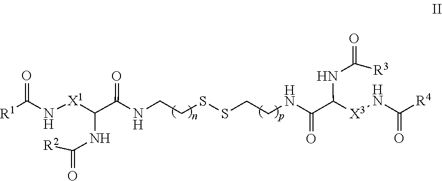

In any embodiments, the compound of Formula I (or IA) is a compound of Formula III,

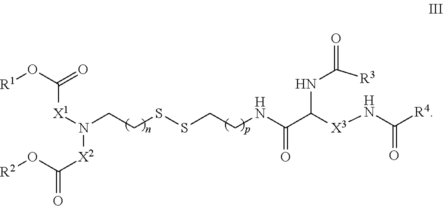

In any embodiments, the present compounds may include 1, 2, or 3 ionizable functional groups. In any embodiments, each ionizable functional group may be independently selected from NH₂, NHR, NR₂, guanidine, imidazole, or amidine, wherein each R is independently an unsubstituted C₁₋₆ alkyl, phenyl, or benzyl group. In any embodiments, at least one ionizable functional group is guanidine.

In another aspect, the present technology provides a nanoparticle comprising any of the disulfide-containing lipopeptides disclosed herein, including but not limited to compounds of Formulas I, II, and III. In any embodiments, the nanoparticle may further include a PEG-lipid, e.g., PEG-DSPE and/or PEG-DMG. In any embodiments, the nanoparticles may further include a structural lipid, e.g., cholesterol and/or β-sitosterol. In any embodiments, the nanoparticles may further include a phospholipid, e.g., DSPC and/or DOPE.

In any embodiments of the present technology, the nanoparticles may include a payload selected from nucleic acids (e.g., DNA, RNA, ASO, mRNA, siRNA, transfer RNA (tRNA), tracrRNA, crRNA, sgRNA, NAD⁺, NADH, etc.), polypeptides, mixture of nucleic acids, mixture of nucleic acids and proteins, and any types of genome editors including CRISPR genome editors in the form of DNA, mRNA, CasX (e.g., X=9, 12, 13, 14, Φ), mRNA/gRNA (e.g., sgRNA, crRNA, or tracrRNA+crRNA), CasX protein/gRNA (e.g., sgRNA, crRNA, or tracrRNA+crRNA) ribonucleoprotein complex (RNP), RNP+ssODN, base editors, primer editors etc.) and TALEN for genome editing in nucleus and/or mitochondria, and any types of epigenetic editors (e.g., dCas9-LSD1/sgRNA, dCas9-TET1/sgRNA etc.)

In another aspect, the present technology includes a method of delivering a payload into an animal cell, e.g., a mammalian cell, the method comprising exposing the animal cell to a nanoparticle described herein.

In another aspect, the present technology includes a method of treating a condition or disorder in a subject that may be ameliorated by a payload selected from RNA, DNA, a nucleotide (e.g., NAD⁺ or NADH), a polypeptide or an RNP, the method comprising administering to the subject an effective amount of a nanoparticle as described herein.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIGS. 1A-1C show characterization data for illustrative embodiments of LNPs, i.e., a lipoplex and RNP-lipoplex or RNP/ssODN-lipoplex nanocomplexes. FIG. 1A shows the hydrodynamic diameters of empty liposomal particles (ELP), RNP-lipoplex, and RNP/ssODN-lipoplex tested by DLS. FIG. 1B shows zeta-potentials of ELP, RNP-lipoplex, and RNP/ssODN-lipoplex (n=3). FIG. 1C shows a TEM image of an RNP-lipoplex. Scale bar: 300 nm.

FIGS. 2A-2F show in vitro studies of the transfection or editing efficiency of an illustrative embodiment of the LNP and biologics complexes where said complexes are lipoplexes. FIG. 2A shows the optimization of the ELP and RNP weight ratio for the RNP-lipoplex nanocoplexes in GFP-expressing HEK 293 cells. (n=3). FIG. 2B shows optimization of the ELP and mRNA weight ratio for the mRNA-lipoplex nanocomplexes in HEK 293 cells. (n=3). FIG. 2C shows optimization of the ELP and DNA weight ratio for the DNA-lipoplex nanocomplexes in HEK 293 cells. (n=3). FIG. 2D shows optimization of the ELP and mRNA weight ratio for the mRNA-lipoplex nanocomplexes in NIH 3T3 cells. (n=3). FIG. 2E shows optimization of the ELP and mRNA weight ratio for the mRNA-lipoplex nanocomplexes in Raw 264.7 cells. (n=3). FIG. 2F shows optimization of the ELP and DNA weight ratio for the DNA-lipoplex nanocoplexes in B78 melanoma cells. (n=3). Statistical significance was calculated via one-way ANOVA with a Tukey post hoc test. *P<0.05. **P<0.01. ***P<0.001. ****P<0.0001. ns, not significant.

FIG. 3 shows agarose gel electrophoresis of an illustrative embodiment of an RNP-lipoplex nanocomplexes at different LNP/RNP weight ratios.

FIG. 4 shows results of cell viability study of an illustrative embodiment of DNA-lipoplex in HEK 293 cells via an MTT assay (n=3). Statistical significance was calculated via one-way ANOVA with a Tukey post hoc test. **P<0.01. ns, no significance.

FIG. 5 shows effects of GSH concentration in the cell culture medium on the editing efficiency of an illustrative embodiment of RNP-lipoplex. Statistical significance was calculated via one-way ANOVA with a Tukey post hoc test. ****P<0.0001. ns, no significance.

FIG. 6 shows intracellular trafficking study of an illustrative embodiment of an RNP-lipoplex at different time points after treatments. The Cas9 RNP was labeled with Atto 550-gRNA (4^{th} column). Cells were stained with LysoTracker Green DND-26 (3^{rd} column) and Hoechst 33342 (2^{nd} column) for endosomes/lysosomes and nuclei, respectively. Scale bar: 25 μm.

FIG. 7A-7E Genome editing mediated by an illustrative embodiment of an RNP-lipoplex in Ai14 mice. FIG. 7A is a schematic of the tdTomato locus in the Ai14 mouse strain. A STOP cassette that consists of three SV40 polyA sequences prevents downstream expression of tdTomato (top). tdTomato expression can be induced after removal of two SV40 polyA sequences using CRISPR-Cas9 ribonucleoprotein containing the sgRNA that targets SV40 PolyA (bottom). FIG. 7B is a schematic illustration of intramuscular injections on Ai14 mice. FIG. 7C shows tdTomato expression in tibialis anterior muscles imaged by IVIS. FIG. 7D shows quantification of tdTomato fluorescence intensity (n=3). Statistical significance was calculated via the t-test. *P<0.05. FIG. 7E shows immunofluorescence staining of tibialis anterior muscle sections from the PBS and RNP-lipoplex injected mice. Muscle sections were stained with anti-RFP antibody for tdTomato and DAPI for nuclei. Scale bar: 100 μm for 20×, 50 μm for 40×.

FIGS. 8A-8D. Genome editing mediated by an illustrative embodiment of RNP/ssODN-lipoplex on mdx mice. FIG. 8A is a schematic illustration of intramuscular injections on mdx mice. FIG. 8B shows results of the four-limb hanging time assay of the PBS injected mdx mice (n=7), RNP/ssODN-lipoplex injected mdx mice (n=9), and untreated wild-type mice (n=5). Data were acquired 28 days after injections. FIG. 8C shows total indel frequency of the PBS or RNP/ssODN-lipoplex treated tibialis anterior muscles of mdx mice measured by sanger sequencing (n=3). FIG. 8D shows immunogenicity of the PBS or RNP/ssODN-lipoplex treated tibialis anterior muscles of mdx mice. Expression levels of cytokines were quantified by RT-qPCR (n=4). Statistical significance was calculated via one-way ANOVA with a Tukey post hoc test (8C) or by the t-test (8D). **P<0.01. ***P<0.001. ns, not significant.

FIGS. 9A-9B show a four-limb hanging time assay of the PBS injected mdx mice (n=7), RNP/ssODN-lipoplex-injected mdx mice (n=9), and untreated wild-type mice (n=5). Data was acquired 14 days (FIG. 9A) and 21 days (FIG. 9B) after injections. Statistical significance was calculated via one-way ANOVA with a Tukey post hoc test. *P<0.05. **P<0.01.

FIG. 10 shows Immunostaining of dystrophin in the tibialis anterior muscle sections from the PBS and RNP/ssODN-lipoplex treated mdx mice and untreated wild-type mice at different distances from the tendon. Muscle sections were stained with anti-dystrophin antibody for dystrophin and DAPI for nuclei. Scale bar: 100 m.

FIGS. 11A-11B show H&E Staining (FIG. 11A) and Masson's staining (FIG. 11B) for the histology analysis of the tibialis anterior muscle sections from the PBS or RNP/ssODN-lipoplex treated mdx mice and untreated wild-type mice. Scale bar: 100 μm.

FIGS. 12A-12C show in vitro studies of the transfection of an illustrative embodiment of the mRNA-loaded LNP in NIH 3T3 cells. FIG. 12A shows the results (percentage of GFP-positive cells) from a screening of the tail structures of the lipopeptides (n=3). FIG. 12B shows results from a study of the disulfide bond function of the lipopeptides (n=3). FIG. 12C shows the screening results for the head structures of the lipopeptides. Statistical significance was calculated via one-way ANOVA with a Tukey post hoc test. ****P<0.0001. NS, not significant.

FIG. 13 shows in vitro studies of the transfection of an illustrative embodiment of four-lipid LNP formulations in NIH 3T3 cells. Statistical significance was calculated via one-way ANOVA with a Tukey post hoc test. ****P<0.0001.

FIG. 14 shows in vitro studies of the transfection of an illustrative example of mRNA-loaded SNP with or without lipopeptide coating in NIH 3T3 cells (n=2). Statistical significance was calculated via one-way ANOVA with a Tukey post hoc test. **P<0.01.

FIG. 15. Schematic illustration of fabrication process of lipopeptide-based lipoplexes and solid LNPs.

FIGS. 16A-16D show optimization of the LNP formulations including the molar ratio of the lipopeptide AC18:1-SS-K(R)R for GFP-mRNA or RFP-DNA transfection in NIH 3T3 cells (n=3). FIG. 16A shows quantification of the percentage of GFP positive cells. FIG. 16B shows quantification of the mean fluorescence intensity of the GFP signal. FIG. 16C shows quantification of the percentage of RFP positive cells. FIG. 16D shows quantification of the mean fluorescence intensity of the RFP signal. Statistical significance was calculated via one-way ANOVA with a Tukey post hoc test. * P<0.05. **P<0.01. ***P<0.001. ns, no significance.

FIGS. 17A-17D show optimization of the LNP formulations including the molar ratio of the lipopeptide AC18:1-SS-K(R)R for Cas9-mRNA/sgRNA or RNP editing in GFP-HEK 293 cells (n=3). The figures show quantification of the percentage of GFP negative cells and the mean fluorescence intensity of the GFP signal for Cas9-mRNA/sgRNA delivery (FIGS. 17A and 17B) and RNP delivery (FIGS. 17C and 17D). Statistical significance was calculated via one-way ANOVA with a Tukey post hoc test. * P<0.05. **P<0.01. ***P<0.001. ****P<0.0001. ns, no significance.

FIGS. 18A-18B show optimization of the mRNA buffer pH for the fabrication of the LNPs based on the lipopeptide AC18:1-SS-K(R)R with various formulations for the delivery of GFP-mRNA in NIH 3T3 cells (n=3). Two days after the treatment, the GFP fluorescence was measured by flow cytometry to assay the transfection efficiency. FIG. 18A shows quantification of the percentage of GFP positive cells. FIG. 18B shows quantification of the mean fluorescence intensity (MFI) of the GFP signal. Statistical significance was calculated via one-way ANOVA with a Tukey post hoc test. *P<0.05. ***P<0.001. ****P<0.0001. ns, no significance.

FIGS. 19A-19B show comparison of the transfection efficiency between 98.5% AC18:1-SS-K(R)R LNP with Dlin-MC3-DMA in NIH 3T3 cells (n=3) (FIG. 19A), and quantification of the mean fluorescence intensity (MFI) of the GFP signal (FIG. 19B). Statistical significance was calculated via one-way ANOVA with a Tukey post hoc test. *P<0.05. **P<0.01. ****P<0.0001.

FIGS. 20A-20B show stability study of 98.5% AC18:1-SS-K(R)R LNP (n=3). Two days after the treatment in NIH 3T3 cells, the gain of GFP fluorescence was measured by flow cytometry to assay the transfection efficiency. FIG. 20A shows quantification of the percentage of GFP positive cells. FIG. 20B shows quantification of the mean fluorescence intensity (MFI) of the GFP signal. Statistical significance was calculated via one-way ANOVA with a Tukey post hoc test. ns, no significance.

FIGS. 21A-21D show stability study of lyophilized 98.5% AC18:1-SS-K(R)R LNP (n=3). Two days after the treatment, the GFP fluorescence was measured by flow cytometry to assay the transfection efficiency. FIG. 21A shows quantification of the percentage of GFP positive cells. FIG. 21B shows quantification of the mean fluorescence intensity (MFI) of the GFP signal. FIG. 21C is a heat map of the percentage of GFP positive cells and FIG. 21D shows the MFI in NIH 3T3 cells treated by lyophilized LNPs with different amounts of sucrose. Statistical significance was calculated via one-way ANOVA with a Tukey post hoc test. **P<0.01. ****P<0.0001. ns, no significance. In each figure, the treatment conditions as shown from left to right are: UT, Lipo 2000, 1×PBS fresh LNP, 0.5×PBS fresh LNP, 0.25×PBS fresh LNP, Lyo 0.75 mg/mL 5% sucrose, Lyo 1.5 mg/mL 5% sucrose, Lyo 3 mg/mL 5% sucrose, Lyo 0.75 mg/mL 10% sucrose, and Lyo 1.5 mg/mL 10% sucrose.

FIGS. 22A-22D show confocal microscopy images of RPE florets from Ai14 mice treated with (FIG. 22A) PBS, (FIG. 22B) 98.5% AC18:1-SS-K(R)R LNP (0.75 mg/mL), (FIG. 22C) 90.5% AC18:1-SS-K(R)R/8% PS LNP (0.75 mg/mL) and (FIG. 22D) Lipoplex (0.5 mg/mL). The RPE florets were harvested seven days after treatment for Cre mRNA delivery. The tdTomato signal in the RPE florets was visualized by CLSM. The experiments were repeated in quadruplicate, and representative images at two magnifications are shown here.

FIGS. 23A-23B show confocal microscopy images of RPE florets from Ai14 mice treated with (FIG. 23A) PBS and (FIG. 23B) 50% AC18:1-SS-K(R)R/8% PS (0.75 mg/mL RNP). Ai14 mouse model greatly underreports the editing efficiency of CRISPR genome editors (e.g., Cas9 RNP or Cas9 mRNA/sgRNA). The RPE florets were harvested two weeks after treatment. The tdTomato signal in the RPE florets was visualized by CLSM. The experiments were repeated in quadruplicate, and representative images at two magnifications are shown here. Note: The Ai14 transgenic mouse model underreports the CRISPR genome editing efficiency.

FIGS. 24A-24C show representative confocal microscopy images of retinal sections for Cre mRNA delivery showing tdTomato, stained with visual arrestin (rods, cones, and outer segments) and DAPI following treatments with (FIG. 24A) PBS, (FIG. 24B) 98.5% AC18:1-SS-K(R)R LNP, and (FIG. 24C) 90.5% AC18:1-SS-K(R)R-8% PS Cre mRNA delivery in Ai14 mice. Pink arrows indicated tdTomato expression in the RPE layer.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In some embodiments, the amide is —NR⁷¹C(O)—(C₁₋₅ alkyl) and the group is termed “alkanoylamino.”

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, aryl aralkyl, heterocyclyl or heterocyclylalkyl group as defined herein. It will be understood that amidines may exist in protonated forms in certain aqueous solutions or mixtures and are examples of charged functional groups herein.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In some embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “amino acid” as used herein refers to alpha-amino acids and includes proteinogenic amino acids (e.g., glycine, leucine, phenylalanine, histidine, arginine, lysine, and the like) as well as non-proteinogenic amino acids (e.g., ornithine, D-arginine, ethylglycine, and the like). An “amino acid residue” as this term is used herein refers to an amino acid covalently linked to one or more amino acids via a peptide and/or isopeptide bond(s).

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3^{rd} Edition, 1999). Which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O⁻. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures, and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R⁸⁵ are independently a positive counterion, e.g., Na⁺, K⁺, ammonium, etc. As such, it will be understood that phosphates are examples of charged functional groups herein.

The term “pyridinyl” refers to a pyridine group or a salt thereof. A pyridinyl may be protonated in certain aqueous solutions or mixtures, and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In some embodiments the sulfide is an alkylthio group, —S-alkyl. R⁸³ includes H or, when the sulfonyl is ionized (i.e., as a sulfonate), a positive counterion, e.g., Na⁺, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

As used herein, “ionizable functional group” refers to a functional group that is partially or completely protonated at physiological pH and therefore is positively charged.

An “isopeptide bond” as used herein refers to an amide bond between an amino/carboxyl group in a sidechain of an amino acid residue and the carboxyl/alpha-amino group of another amino acid residue. For example an amide bond between the 6-amino group of lysine and the carboxyl group of argininie is an isopeptide bond.

A “peptide bond” as used herein refers to an amide bond between the carboxyl of one amino acid residue and the alpha-amino group of another amino acid residue.

As used herein, “ribonucleoprotein” or “RNP” refers to a complex between an RNA-binding protein and RNA in which the RNA binds specifically (as opposed to non-specific binding) to the protein. Examples of ribonucleoproteins include CRISPR-associated proteins, e.g., Cas9, Cas12, Cas13, Cas14 and CasΦ.

As used herein, “Cas9” and “Cas9 polypeptide” refer to the Cas9 proteins and variants thereof that, when complexed with RNA (i.e., sgRNA, or crRNA and tracrRNA), have nuclease activity. Likewise, “Cas12” refers to the Cas12 proteins and variants thereof, that, when complexed with crRNA, have nuclease activity. “Cas13” refers to the Cas13 proteins and variants thereof, that have nuclease activity when complexed with RNA (i.e., crRNA). “Cas14” refers to the Cas14 proteins and variants thereof that, when complexed with RNA (i.e., sgRNA, or crRNA and tracrRNA), have nuclease activity. “CasΦ” refers to the CasΦ proteins and variants thereof having nuclease activity, when complexed with crRNA. The variants may include deletions or additions, such as, e.g., addition of one, two, or more nuclear localization sequences (such as from JoeSV40 and others known in the art), e.g., 1, 2, 3, 4, 5, 6, 7, 8, 9, 10 such sequences or a range between and including any two of the foregoing values.

In any embodiments the Cas9 polypeptide is a Cas9 protein found in a type II CRISPR-associated system. Suitable Cas9 polypeptides that may be used in the present technology include, but are not limited to Cas9 protein from Streptococcus pyogenes (SpCas9), F. novicida (FnCas9), S. aureus (SaCas9), S. thermophiles (St1Cas9), N. meningitidis (NmeCas9), and variants thereof. In any embodiments, the Cas9 polypeptide is a wild-type Cas9, a nickase, or comprises a nuclease inactivated (dCas9) protein.

In any embodiments the Cas12 polypeptide is a Cas12 protein found in a type V CRISPR-associated system. Suitable Cas12 polypeptides that may be used in the present technology include, but are not limited to Cas12 protein from Lachnospiraceae bacterium MA2020 (LbCas12a), Acidaminococcus sp. BV3L6 (AsCas12a), Bacillus hisaishi (BhCas12b), and variants thereof. In any embodiments, the Cas12 polypeptide is a wild-type Cas12, a nickase, or comprises a nuclease inactivated (dCas12) protein.

In any embodiments the Cas13 polypeptide is a Cas13 protein found in a type VI CRISPR-associated system. Suitable Cas13 polypeptides that may be used in the present technology include, but are not limited to Cas13 protein from Leptotrichia wadei (LwaCas13a), Prevotella sp. P5-125 (PspCas13b), Ruminococcus flavefaciens (RfxCas13d), and variants thereof. In any embodiments, the Cas13 polypeptide is a wild-type Cas13, a nickase, or comprises a nuclease inactivated (dCas13) protein.

In any embodiments the Cas14 polypeptide is a Cas14 protein found in a type II CRISPR-associated system. Suitable Cas14 polypeptides that may be used in the present technology include, but are not limited to Cas14 protein from uncultivated archaea, and variants thereof. In any embodiments, the Cas14 polypeptide is a wild-type Cas14, a nickase, or comprises a nuclease inactivated (dCas14) protein.

In any embodiments the CasΦ polypeptide is a CasΦ protein found in a type II CRISPR-associated system. Suitable CasΦ polypeptides that may be used in the present technology include, but are not limited to CasΦ protein from biggiephage clade and variants thereof. In any embodiments, the CasΦ polypeptide is a wild-type CasΦ, a nickase, or comprises a nuclease inactivated (dCasΦ) protein.

Cas9, Cas12, Cas13, Cas14, and CasΦ polypeptides also include fusion proteins containing such Cas9, Cas12, Cas13, Cas14, and CasΦ proteins and variants thereof. The fused proteins may include those that modify the epigenome or control transcriptional activity. In any embodiments, the Cas9 polypeptide is a fusion protein comprising dCas9. In any embodiments, the Cas12 polypeptide is a fusion protein comprising dCas12. In any embodiments, the Cas13 polypeptide is a fusion protein comprising dCas13. In any embodiments, the Cas14 polypeptide is a fusion protein comprising dCas14. In any embodiments, the CasΦ polypeptide is a fusion protein comprising dCasΦ. In any embodiments, the fusion protein comprises a transcriptional activator (e.g., VP64), a transcriptional repressor (e.g., KRAB, SID) a nuclease domain (e.g., FokI), base editor (e.g., adenine base editors, ABE), a recombinase domain (e.g., Hin, Gin, or Tn3), a deaminase (e.g., a cytidine deaminase or an adenosine deaminase) or an epigenetic modifier domain (e.g., TET1, p300).

In any embodiments, Cas9, Cas12, Cas13, Cas14, and CasΦ includes variants with at least 85% sequence identity, at least 90% sequence identity, at least 95% sequence identity, or even 96%, 97%, 98%, or 99% sequence identity to the wild type Cas9, Cas12, Cas13, Cas14, or CasΦ, respectively. Accordingly, a wide variety of CasX (e.g., X=9, 12, 13, 14, Φ) RNPs or CasX and guide RNA (e.g., sgRNA, crRNA, or tracrRNA+crRNA) encoded plasmids, or CasX encoded mRNA with guide RNA (e.g., sgRNA, crRNA, or tracrRNA+crRNA) may be used as formation of the present LNPs is not sequence dependent so long as the biomeluecules can be encapsulated by the LNP and/or electrostatically bound to the LNP of the present technology. Other suitable Cas systems may be found in Karvelis, G. et al. “Harnessing the natural diversity and in vitro evolution of Cas9 to expand the genome editing toolbox,” Current Opinion in Microbiology 37: 88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein in their entirety. Other suitable Cas12 proteins may be found in Makarova, Kira S., et al. “Evolutionary classification of CRISPR-Cas systems: a burst of class 2 and derived variants.” Nature Reviews Microbiology 18.2 (2020): 67-83; Strecker, Jonathan, et al. “Engineering of CRISPR-Cas12b for human genome editing.” Nature Comm. 10.1 (2019): 1-8; and Yan, Winston X., et al. “Functionally diverse type V CRISPR-Cas systems.” Science 363.6422 (2019): 88-91, each of which is incorporated by reference herein in their entirety. Other suitable Cas13 proteins may be found in O'Connell, Mitchell R. “Molecular mechanisms of RNA targeting by Cas13-containing type VI CRISPR-Cas systems.” J. Mol. Biol. 431.1 (2019): 66-87, each of which is incorporated by reference herein in their entirety.

“Molecular weight” as used herein with respect to polymers refers to number-average molecular weights (Mₙ) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

A “cell penetrating peptide” (CPP), also referred to as a “protein transduction domain” (PTD), a “membrane translocating sequence,” and a “Trojan peptide”, refers to a short peptide (e.g., from 4 to about 40 amino acids) that has the ability to translocate across a cellular membrane to gain access to the interior of a cell and to carry into the cells a variety of covalently and noncovalently conjugated cargoes, including the present nanoparticles, including with their payloads. CPPs are typically highly cationic and rich in arginine and lysine amino acids. Examples of such peptides include TAT cell penetrating peptide (GRKKRRQRRRPQ); MAP (KLALKLALKALKAALKLA); Penetratin or Antenapedia PTD (RQIKWFQNRRMKWKK); Penetratin-Arg: (RQIRIWFQNRRMRWRR); antitrypsin (358-374): (CSIPPEVKFNKPFVYLI); Temporin L: (FVQWFSKFLGRIL-NH2); Maurocalcine: GDC(acm) (LPHLKLC); pVEC (Cadherin-5): (LLIILRRRIRKQAHAHSK); Calcitonin: (LGTYTQDFNKFHTFPQTAIGVGAP); Neurturin: (GAAEAAARVYDLGLRRLRQRRRLRRERVRA); Penetratin: (RQIKIWFQNRRMKWKKGG); TAT-HA2 Fusion Peptide: (RRRQRRKKRGGDIMGEWGNEIFGAIAGFLG); TAT (47-57) Y(GRKKRRQRRR); SynB1 (RGGRLSYSRRRFSTSTGR); SynB3 (RRLSYSRRRF); PTD-4 (PIRRRKKLRRL); PTD-5 (RRQRRTSKLMKR); FHV Coat-(35-49) (RRRRNRTRRNRRRVR); BMV Gag-(7-25) (KMTRAQRRAAARRNRWTAR); HTLV-II Rex-(4-16) (TRRQRTRRARRNR); HIV-1 Tat (48-60) or D-Tat (GRKKRRQRRRPPQ); R9-Tat (GRRRRRRRRRPPQ); Transportan (GWTLNSAGYLLGKINLKALAALAKKIL chimera); SBP or Human P1 (MGLGLHLLVLAAALQGAWSQPKKKRKV); FBP (GALFLGWLGAAGSTMGAWSQPKKKRKV); MPG (ac-GALFLGFLGAAGSTMGAWSQPKKKRKV-cya (wherein cya is cysteamine)); MPG(ΔNLS) (ac-GALFLGFLGAAGSTMGAWSQPKSKRKV-cya); Pep-1 or Pep-1-Cysteamine (ac-KETWWETWWTEWSQPKKKRKV-cya); Pep-2 (ac-KETWFETWFTEWSQPKKKRKV-cya); Periodic sequences, Polyarginines (RxN (4<N<17) chimera); Polylysines (KxN (4<N<17) chimera); (Raca)6R; (Rabu)6R; (RG)6R; (RM)6R; (RT)6R; (RS)6R; R10; (RA)6R; and R7.

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR© dyes (e.g., ALEXA FLUOR© 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

The phrase “lipid nanoparticle” (LNP) refers to a particle with a sub-micron hydrodynamic diameter (as measured by DLS) and comprising one or more of the herein-disclosed lipopeptides, including disulfide-containing lipopeptides and/or lipopeptides without disulfides, and optionally PEG-lipids, and/or structural lipids, and/or phospholipids. The LNPs may be liposomes, e.g., a liposome containing or comprising a payload (referred to herein as a lipoplex) or a solid LNP lacking a lipid bilayer. The present LNPs are capable of binding and/or complexing and/or encapsulating, and/or otherwise carrying and delivering nucleotides, nucleic acids and/or gene-editing systems to cells.

The phrase “PEG-lipid” refers to a molecule that comprises a polyethylene glycol group covalently bound to a lipid group, e.g., a fatty acid or ester of a fatty acid such as a glyceryl mono- or di-ester of a fatty acid. Examples of the PEG-lipid include PEG-DSPE, PEG-DOPS, PEG-DMG, or the like.

The phrase “phospholipid” refers to a molecule that comprises a phosphate group covalently bound to lipid tails derived from fatty acids. Examples of the phospholipid include DSPC, and/or DOPE and/or the like.

The phrase “structural lipid” refers to the cholesterol or its analogues, which contains a central sterol nucleus made of four hydrocarbon rings, a hydroxyl group, and a linear or branched hydrocarbon tail. In any embodiments, structural lipid may be cholesterol and/or β-sitosterol, and/or the like.

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. Examples of such ligands include GE11 peptide, anti-EGFR nanobody, cRGD ((cyclo (RGDfC)), KE108 peptide, octreotide, glucose, all-trans-retinoic acid (ATRA), RVG peptide (YTIWMPENPRPGTPCDIFTNSRGKRASNG), mannose, galactose, rabies virus glycoprotein (RVG) peptide, mannitol, folic acid, prostate-specific membrane antigen (PSMA) aptamer, TRC105, a human/murine chimeric IgGI monoclonal antibody, cholera toxin B (CTB), hyaluronic acid, and N-acetylgalactosamine (GalNAc). Additional examples of such ligands include Rituximab, Trastuzumab, Bevacizumab, Alemtuzumab, Panitumumab, RGD, DARPins, RNA aptamers, DNA aptamers, analogs of folic acid and other folate receptor-binding molecules, lectins, other vitamins, amino acids, peptide ligands identified from library screens, tumor-specific peptides, tumor-specific aptamers, tumor-specific carbohydrates, tumor-specific monoclonal or polyclonal antibodies, Fab or scFv (i.e., a single chain variable region) fragments of antibodies such as, for example, an Fab fragment of an antibody directed to EphA2 or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selecting, steroid hormones, Arg-Gly-Asp containing peptides, microtubule-associated sequence (MTAS), various galectins, δ-opioid receptor ligands, cholecystokinin A receptor ligands, ligands specific for angiotensin AT1 or AT2 receptors, peroxisome proliferator-activated receptor γ ligands, β-lactam antibiotics, small organic molecules including antimicrobial drugs, and other molecules that bind specifically to a receptor preferentially expressed on the surface of targeted cells or on an infectious organism, or fragments of any of these molecules. In certain embodiments, hybrid targeting ligands may be presented on the LNP. For example, the RVG peptide and glucose can be presented on the LNP simultaneously.

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include EGFR, αᵥβ₃ integrin, somatostatin receptor, folate receptor, prostate-specific membrane antigen, CD105, mannose receptor, estrogen receptor, and GM1 ganglioside.

In one aspect, the present technology provides a compound of Formula I,

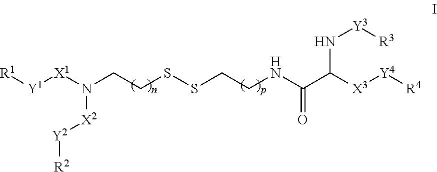

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently absent, C(O)O, or C(O)NH;
Y³ is absent, C(O), C(O)O, or C(O)NH;
Y⁴ is C(O)O, or C(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid, peptide, or isopeptide; and further provided that if one of R³ and R⁴ is absent, the other is present; and
n and p are each independently 1, 2, 3, 4, or 5.

In any embodiments of the compounds disclosed herein (including but not limited to compounds of Formula I or any of those below), R³ and R⁴ are independently selected from an amino acid, a peptide or isopeptide comprising 2, 3, or 4 amino acid residues. In any embodiments, Y³ is absent and R³ is an amino acid residue bearing one or two ionizable functional groups, e.g., amine and guanideine. In any embodiments Y³ is absent and R³ is arginine (linked via a peptide bond to its carbonyl group). In any embodiments, R³ is absent and R⁴ is present. In any embodiments R⁴ is present and R⁴ is absent. In any embodiments, R³ and R⁴ are present and independently selected from an amino acid residue, a peptide or isopeptide comprising 2-10 amino acid residues, or a C₁₋₁₂ alkyl group, each of which is optionally substituted with 1, 2, or 3 ionizable functional groups. In any embodiments, at least one of R³ and R⁴ is substituted with an ionizable functional group.

In some embodiments, the present technology provides a compound of Formula IA,

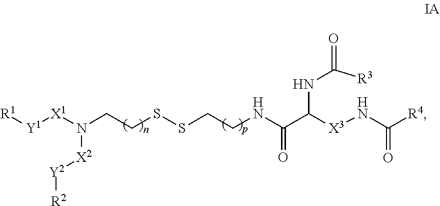

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
n and p are each independently 1, 2, 3, 4, or 5.

In any embodiments, the compound of Formula I may be a compound of Formula II,

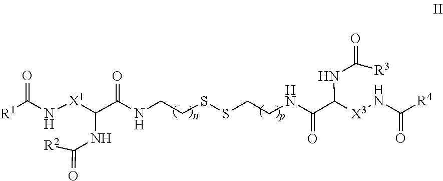

wherein the variables X¹, X³, R¹, R², R³, R⁴, n and p may have any of the values disclosed herein, e.g., as disclosed above in Formula I.

In any embodiments, the compound of Formula II may be a compound of Formula IIA,

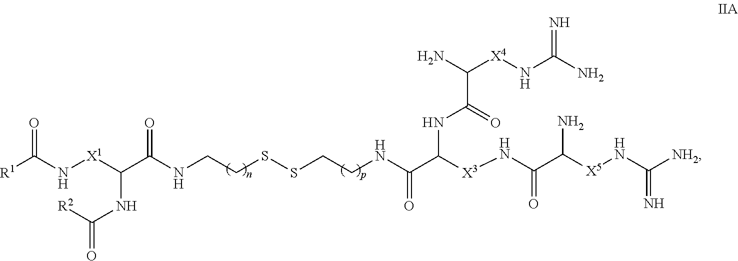

wherein
X⁴ and X⁵ are independently selected from C₁₋₆ alkylene or C₂₋₆ alkenylene groups and the remaining variables may have any of the values disclosed herein, including but not limited to the those disclosed for Formula I above.

In any embodiments, the compound of Formula I may be a compound of Formula III,

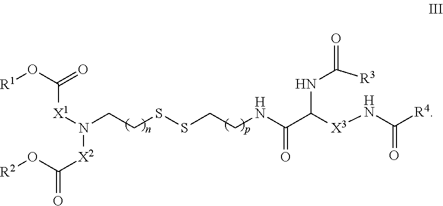

In any embodiments of the compounds disclosed herein (including but not limited to compounds of Formulas I, IA, II, IIA, III, IV, IVA, and IVB), X¹ and X² are independently selected from unsubstituted C₁₋₄ alkylene or C₂₋₄ alkenylene. In any embodiments, X¹ and X² are each butylene.

In any embodiments of the compounds disclosed herein (including but not limited to compounds of Formulas I, IA, II, IIA, III, IV, IVA, and IVB), R¹ and R² may be independently selected from unsubstituted C₁₂₋₂₄ alkyl or C₁₂₋₂₄ alkenyl groups. In any embodiments, R¹ and R² may be independently selected from unsubstituted C₁₆₋₂₀ alkyl or C₁₆₋₂₀ alkenyl groups. In any embodiments, R¹ and R² are independently a C₁₈ alkyl group or a C₁₈ alkenyl group having 1 or 2 carbon-carbon double bonds.

In any embodiments of the compounds disclosed herein (including but not limited to compounds of Formulas I, IA, II, IIA, III, IV, IVA, and IVB), R³ and R⁴ may be independently selected from C₂₋₄ alkyl groups substituted with 1, 2, or 3 ionizable functional groups. In any embodiments, R³ and R⁴ may be substituted with 1 or 2 ionizable functional groups. The ionizable functional groups may be independently selected from NH₂, NHR, NR₂, guanidine, imidazole, or amidine, wherein each R is independently an unsubstituted C₁₋₆ alkyl, phenyl, or benzyl group. In any embodiments, the ionizable functional group may be guanidine or amidine. In any embodiments, at least one ionizable functional group of R³ and/or R⁴ is guanidine. In any embodiments, R³ and R⁴ may be each substituted with an NH₂ group and a guanidine group.

In any embodiments of the compounds disclosed herein (including but not limited to compounds of Formulas I, II, IIA, and III), the compound is selected from the group consisting of

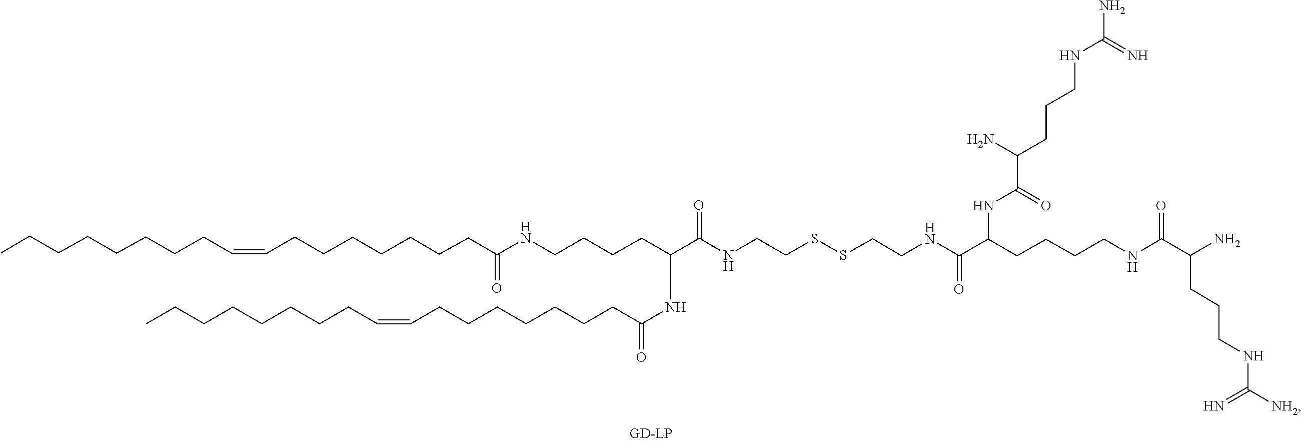 [SMILES: CCCCCCCC/C=C\CCCCCCCC(=O)NCCCCC(NC(=O)CCCCCCC/C=C\CCCCCCCC)C(=O)NCCSSCCNC(=O)C(CCCCNC(=O)C(N)CCCNC(=N)N)NC(=O)C(N)CCCNC(=N)N]

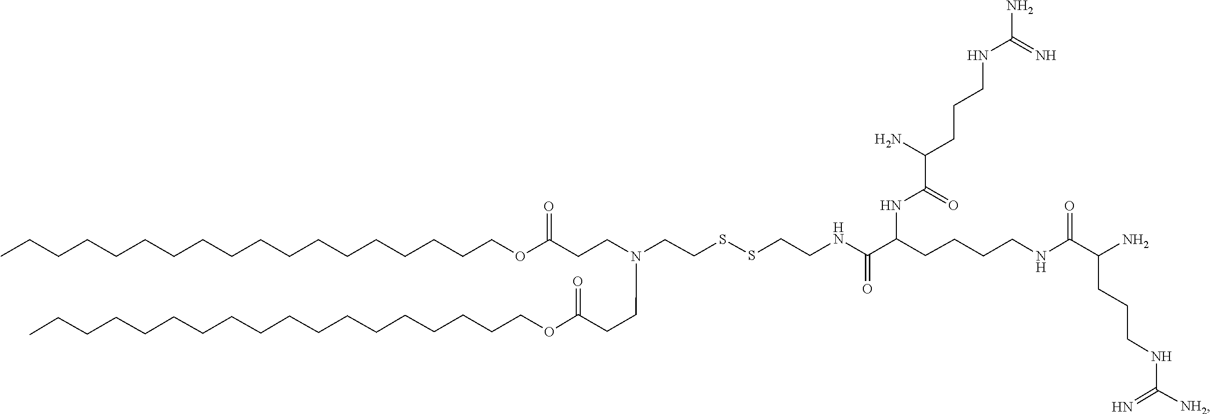 [SMILES: CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC]

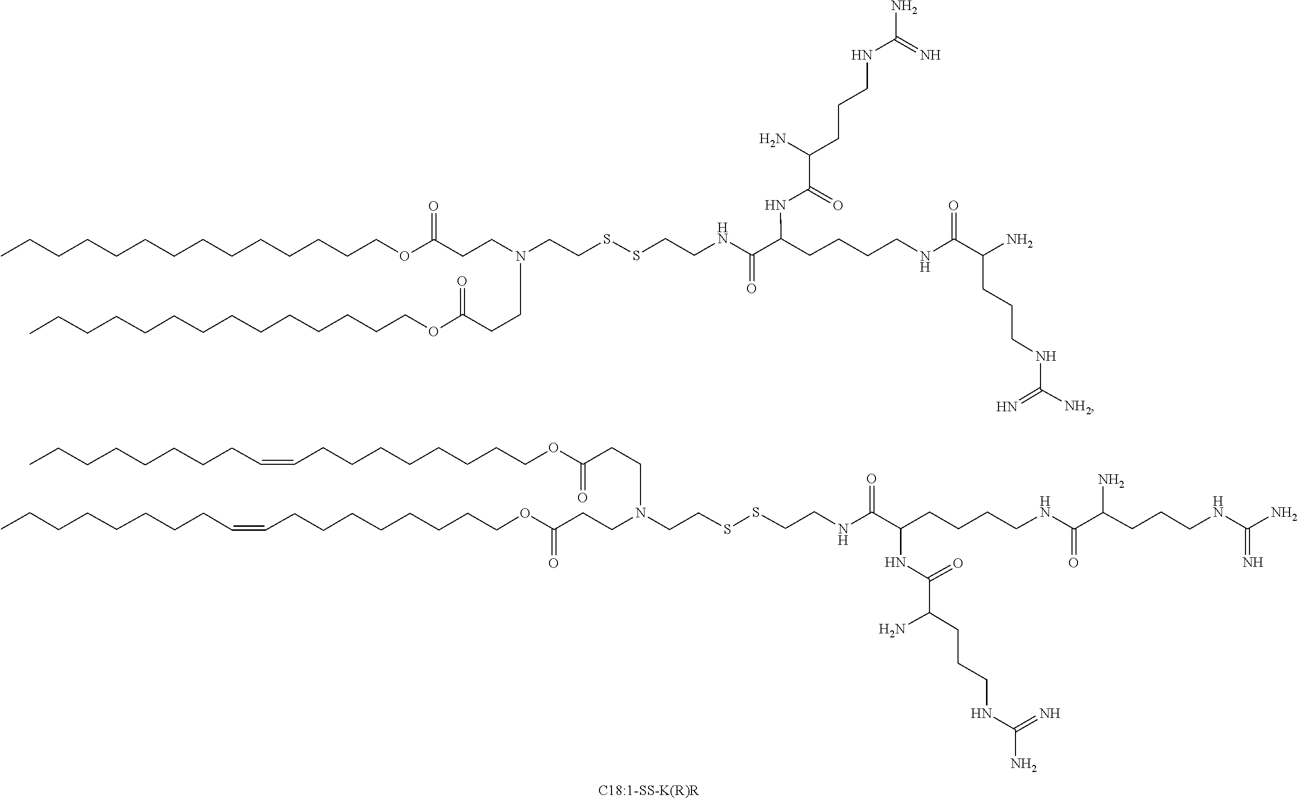 [SMILES: CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC ; CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC]

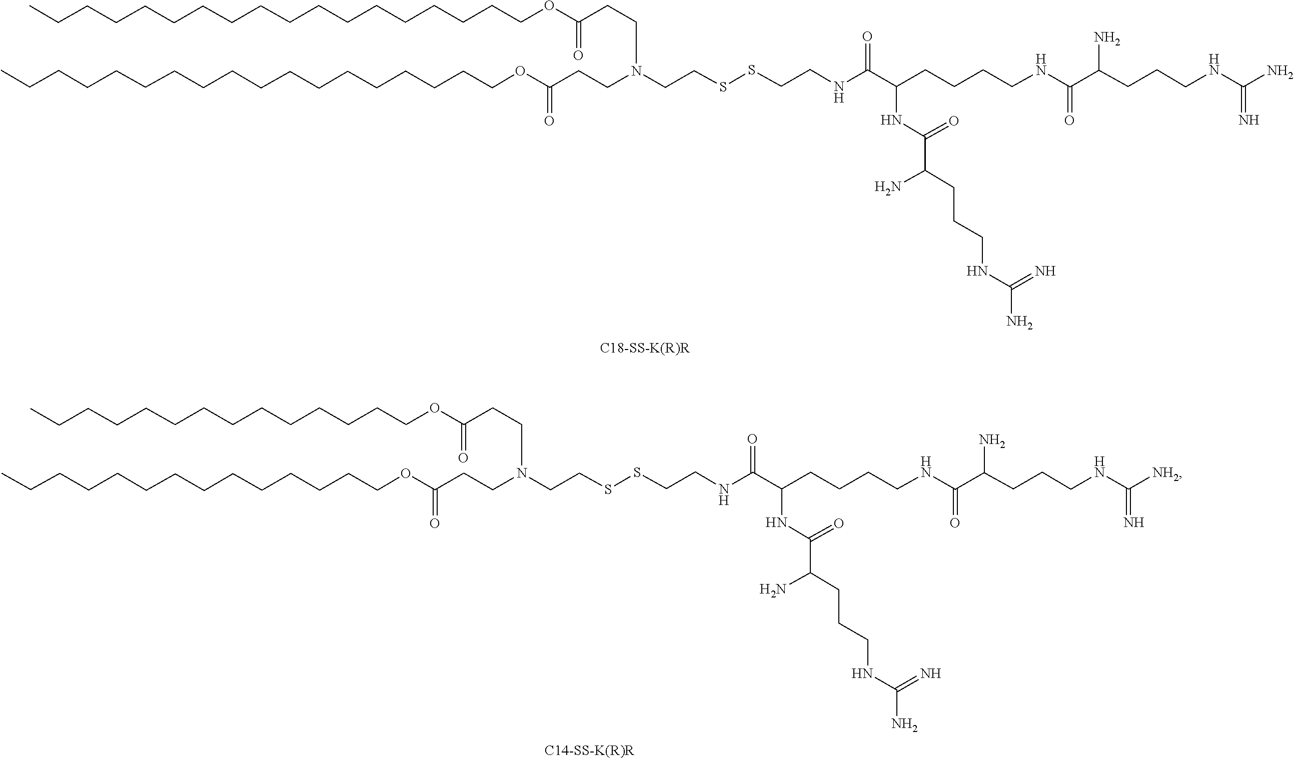 [SMILES: CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC ; CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC ; "C18-SS-K(R)R" ; "C14-SS-K(R)R"]

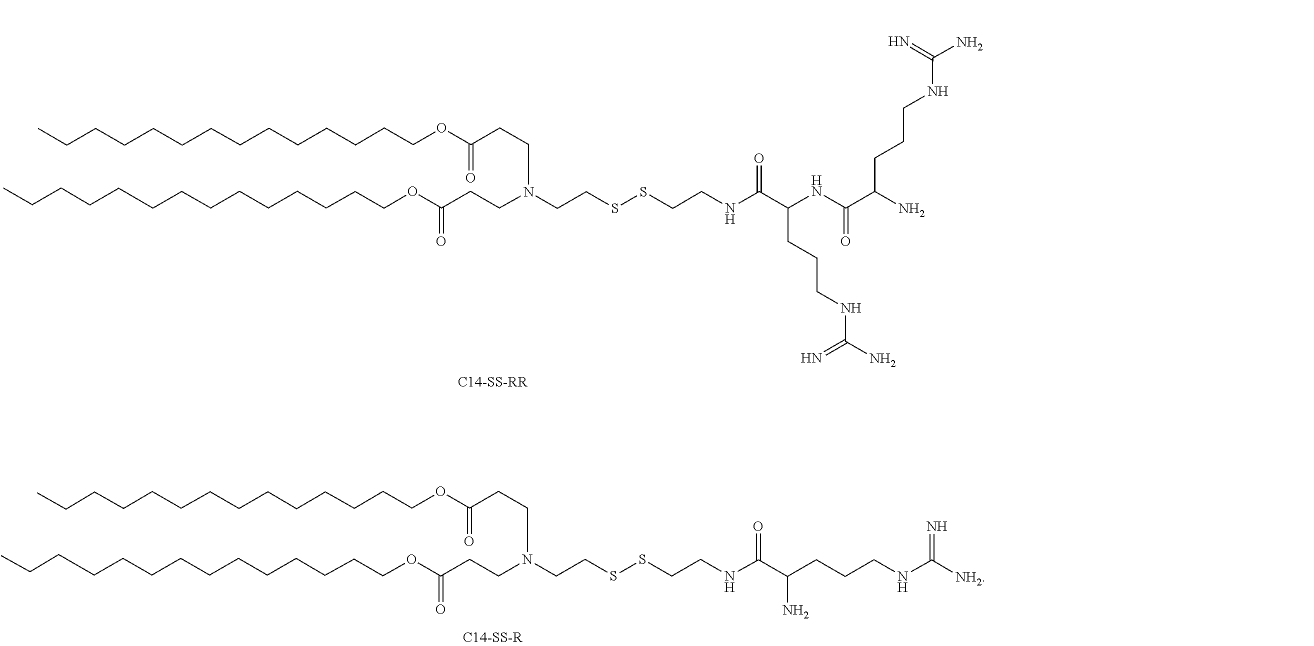 [SMILES: CCCCCCCCCCCCCCCOC(=O)CCN(CCSSCCNC(=O)C(N)CCCNC(=N)N)CCC(=O)OCCCCCCCCCCCCCC ; CCCCCCCCCCCCCCCOC(=O)CCN(CCSSCCNC(=O)C(CCCNC(=N)N)NC(=O)C(N)CCCNC(=N)N)CCC(=O)OCCCCCCCCCCCCCC ; "C14-SS-RR"]

In another aspect, the present technology provides a compound of Formula IV,

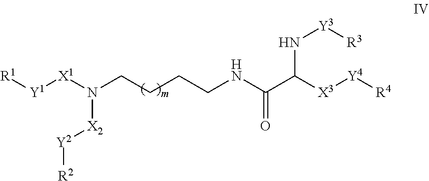

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently absent, C(O)O, or C(O)NH;
Y³ is absent, C(O), C(O)O, or C(O)NH;
Y⁴ is C(O)O, or C(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid, peptide, or isopeptide; and further provided that if one of R³ and R⁴ is absent, the other is present; and
m is 1, 2, 3, 4, 5, 6, 7, 8, 9, 10 or 11.

In any embodiments of the compounds disclosed herein (including but not limited to compounds of Formula IV), R³ and R⁴ are independently selected from an amino acid, a peptide or isopeptide comprising 2, 3, or 4 amino acid residues. In any embodiments, Y³ is absent and R³ is an amino acid residue bearing one or two ionizable functional groups, e.g., amine and guanideine. In any embodiments Y³ is absent and R³ is arginine (linked via a peptide bond to its carbonyl group). In any embodiments, R³ is absent and R⁴ is present. In any embodiments R⁴ is present and R⁴ is absent. In any embodiments, R³ and R⁴ are present and independently selected from an amino acid residue, a peptide or isopeptide comprising 2-10 amino acid residues, or a C₁₋₁₂ alkyl group, each of which is optionally substituted with 1, 2, or 3 ionizable functional groups. In any embodiments, at least one of R³ and R⁴ is substituted with an ionizable functional group. In any embodiments, m is 1, 2, 3, 4, or 5. In any embodiments, m is 1, 2 or 3.

In some embodiments, the present technology provides a compound of Formula IVA,

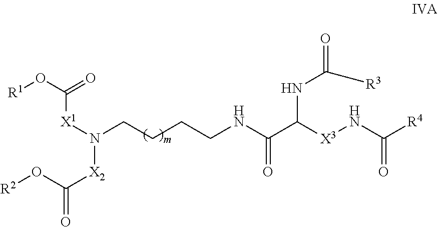

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
m is 1, 2, 3, 4, 5, 6, 7, 8, 9, 10 or 11.

In some embodiments, the present technology provides a compound of Formula IVB,

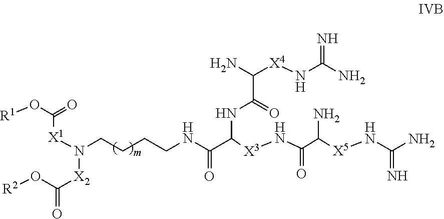

wherein
X⁴ and X⁵ are independently selected from C₁₋₆ alkylene or C₂₋₆ alkenylene groups and the remaining variables may have any of the values disclosed herein, including but not limited to the those disclosed for Formula IV above.

In any embodiments of the compounds disclosed herein (including but not limited to compounds of Formulas IV, IVA, and IVB), the compound is selected from the group consisting of

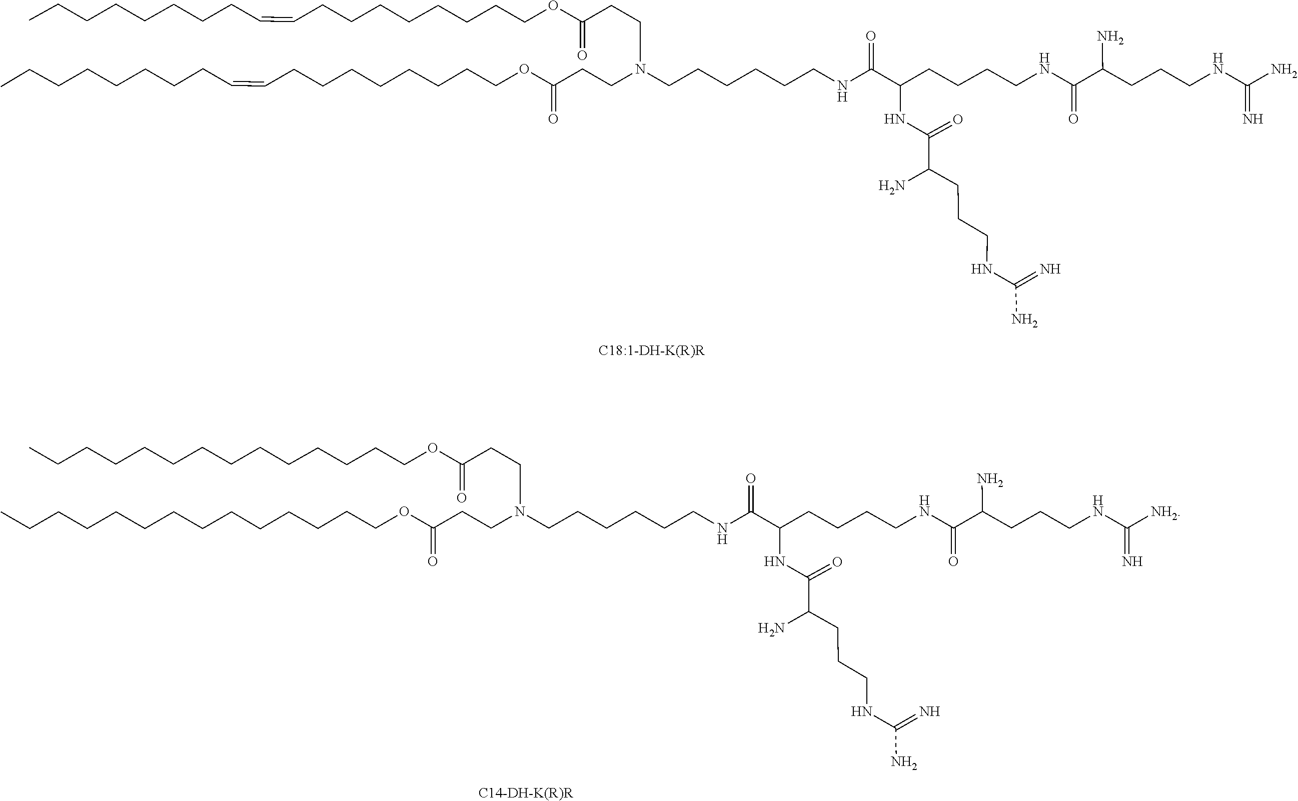

In still another aspect, the present technology provides lipid nanoparticles (LNP) including any of the compounds (e.g., lipopeptides of any one of Formula I, II, IIA, and/or III) disclosed herein. The LNP may be a lipoplex, a solid LNP, or the like. The nanoparticle may include at least 50 mol % of the lipids making up the nanoparticle, such as 50 mol % to 99.5 mol % of the lipids. For example, the nanoparticle may include of of about 50, 55, 60, 65, 70, 75, 80, 85, 90, 91, 92, 93, 94, 95, 95.5, 96, 96.5, 97, 97.5, 98, 98.5, 99, or 99.5 mol % or a range between and including any of the forgoing. For example, the LNP (e.g., lipoplex or solid LNP) may include 80 mol % to 99 mol % of a compound (e.g., a lipopeptide of Formula I) disclosed herein. In any embodiments the LNP may include about 98.5 mol % of a compound disclosed herein such as a compound of Formula I.

In any embodiments, the LNP (including lipoplex or solid LNP) may further include a PEG-lipid. In any embodiments, the PEG-lipid may be selected from any suitable PEG-lipid such as, but not limited to In any embodiments, the PEG-lipid may be 1,2-distearoyl-sn-glycero-3-phosphoethanolamine-N-[carbonyl-methoxy(polyethylene glycol)] (PEG-DSPE), 1,2-dioleoyl-sn-glycero-3-phospho-L-serine-[carbonyl-methoxy(polyethylene glycol)] (PEG-DOPS), 1,2-dipalmitoylphosphatidylcholine-[carbonyl-methoxy(polyethylene glycol)] (PEG-DPPC), and the like. In any embodiments, the LNP may further include PEG-DSPE and/or PEG-DMG. The weight ratio of lipopeptide (e.g., of any one of Formula I, II, IIA, and/or III) to PEG-lipid may be from 100:1 to 1:1. For example, the weight ratio may be any of 100:1, 99:1, 98:1, 95:1, 90:1, 85:1, 80:1, 75:1, 70:1, 65:1, 60:1, 50:1, 45:1, 40:1, 35:1, 30:1, 25:1, 23:1, 22:1, 21:1, 20:1, 19:1, 18:1, 17:1, 16:1, 15:1, 12:1, 10:1, 9:1, 5:1, 4:1, 3:1, 2:1, or 1:1 or a range between and including any two of the foregoing values such as 50:1 to 2:1, or 25:1 to 5:1, or 24:1 to 9:1.

The PEG group in a PEG lipid is a hydrophilic polymer comprising repeating ethylene oxide subunits. The PEG polymeric chains (i.e., PEG groups) may be attached directly or through a linker to the lipid group. Each PEG terminates in one of various groups that, e.g., may be selected from a targeting ligand, OH, O—(C₁₋₆)alkyl, NH₂, COOH, maleimide, CPP, biotin or a dye. In some embodiments the PEG terminates in OH, NH₂, or O—(C₁₋₆)alkyl, and in still others the PEG terminates in in an OC₁₋₃ alkyl group. In still other embodiments, the PEG terminates in a targeting ligand. The targeting ligand may be selected from the group consisting of a cofactor, carbohydrate, peptide, antibody, nanobody, or aptamer. In other embodiments, the targeting ligand is selected from the group consisting of folic acid, mannose, GE11, cRGD, KE108, octreotide, TAT cell penetrating peptide, PSMA aptamer, TRC105, 7D12 nanobody, all-trans retinoic acid (ATRA), 11-cis-retinal (11cRal), CTB, and N-acetylgalactosamine (GalNAc).

Typically, each PEG group in a PEG lipid has 11 to 115 repeat units or a molecular weight of about 500 to about 5,000 Da. Suitable molecular weights for each PEG group of the PEG-lipid include about 500, about 1,000, about 1,500, about 2,000, about 2,500, about 3,000, about 4,000, about 5,0000 Da, or a range between and including any two of the foregoing values (e.g., about 500 to about 3,500 Da or about 1,500 to about 2,500 Da). In any embodiments, the PEG group has a molecular weight of about 2,000 Da.

In any embodiments, the LNP may further include a structural lipid, e.g., cholesterol and/or β-sitosterol.

In any embodiments, the LNP may further include a phospholipid. In any embodiments, the phospholipid may be DSPC (1,2-distearoyl-sn-glycero-3-phosphocholine), DSPE (1,2-distearoyl-sn-glycero-3-phosphocholine), DPPC (1,2-dipalmitoylphosphatidyl-choline), DOPS (1,2-dioleoyl-sn-glycero-3-phospho-L-serine), DOPE (1,2-dioleoyl-sn-glycero-3-phosphoethanolamine) or a mixture of any two or more thereof. In any embodiments, the phospholipid may be DSPC, DOPS and/or DOPE.

In any embodiments, the present LNP may include a payload to be delivered into a cell. The payload may be any suitable biomacromolecule, including any described herein, and is typically for therapeutic use. The biomacromolecule may be encapsulated by the LNP and/or electrostatically bound to the LNP. For example, the payload may be selected from DNA, RNA, a nucleotide (e.g., NAD⁺ or NADH), a polypeptide (e.g., an enzyme or other proteins), a ribonucleoprotein complex (RNP), or a combination of any two or more thereof. In any embodiments, the payload may be selected from the group consisting of RNP, plasmid DNA (pDNA), single-stranded donor oligonucleotide (ssODN), antisense oligonucleotides (ASO), NAD⁺, NADH, complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof. In any embodiments, the payload may be Cas9 RNP, Cas9 RNP+ssODN, where ssODN serves as a repair template, RNP+donor DNA up to 2 kb, and other Cas9-based protein/nucleic acid complexes. It will be appreciated that with the present nanoparticles, Cas9 or RNP need not be conjugated to any repair template as either may simply be mixed with the desired polynucleic acid instead during the nanoparticle formation process. NLS peptides may be used to direct biomacromolecules to the nucleus if desired. For example, polynucleic acids as described herein as well as proteins such as Cas9 or RNP+donor DNA complexes may be covalently tagged (i.e., conjugated) with NLS peptides using techniques well known in the art. Likewise, the biomolecule (e.g., biomacromolecule) may be tagged with a mitochondrial localization signal peptide to direct the biomolecule to the mitochongdria if desired. In any embodiments, the payload may be a mixture of RNAs, e.g., an mRNA for a Cas polypeptide and an sgRNA. Thus, in some embodiments, the payload is a mixture of Cas9 mRNA and an sgRNA, or a Cas12 mRNA and an sgRNA, or a Cas13 mRNA and an sgRNA, or a Cas14 mRNA and an sgRNA, or a CasΦ mRNA and an sgRNA. In any embodiments, they payload may be a plasmid encoding a Cas polypeptide and an sgRNA, e.g., a plasmid encoding Cas9, Cas12, Cas13, Cas14, or CasΦ and an sgRNA. In any embodiments, the payload may be selected from a plasmid or mRNA of transcription activator-like effector nucleases (TALEN).

The present LNPs may have a payload loading content of from about 1 wt % to about 30 wt %, e.g., about 1 wt %, about 5 wt %, about 10 wt %, about 12 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 30 wt % or a range between and including any two of the foregoing values. Thus, in any embodiments, the loading content of the LNP may be, e.g., from about 5 wt % to about 25 wt %, about 10 wt % to about 25 wt %, or about 15 or 18 wt % to about 22 or 24 wt %. Loading efficiency of the present LNPs with payloads described herein is high. In any embodiments, the loading efficiency may be greater than 80%, greater than 85%, or even greater than 90%, e.g., 80%, 85%, 90%, 95%, 99% or a range between and including any two of the foregoing values.

In any embodiments, the biomolecule (e.g., biomacromolecule) may be tagged with an imaging agent, e.g., a dye as described herein. Alternatively, an imaging agent may be incorporated into a lipid in the LNP. The imaging agent (e.g., dye) may be attached to a lipid in the LNP via covalent bonds. By way of a non-limiting example, the bonds may be amide bonds, N—C bonds, imino bonds and the like.

In any embodiments, the present compositions and may further include one or more of a targeting ligand, a CPP, biotin, or an imaging agent attached to the surface of the LNP. The targeting ligands and other groups to be attached typically have a reactive group such as an electrophile or active ester or the like which can react with, e.g., a nucleophilic group on the PEG-lipid such as, but not limited to amino groups. Other amide-bond forming methods or click chemistry may be used join the targeting ligand, CPP, biotin or imaging agent to the nanoparticle.

The present LNPs may be roughly sphere-shaped or may have a more elongated shape. Nevertheless, the “average diameter” of the present LNPs means the average hydrodynamic diameter and ranges from about 50 nm to about 800 nm. Thus, the present LNPs may have an average hydrodynamic diameter of 50, 75, 100, 125, 150, 175, 200, 225, 250, 300, 350, 400, 450, 500, 600, 700, or 800 nm or a range between and including any two of the foregoing values. In any embodiments herein, the LNPs may have an average hydrodynamic diameter of 100 to 800 nm or even 100 nm to 500 nm. When filled or complexed with a payload, the average hydrodynamic diameter is typically a littler larger then the unfilled/uncomplexed LNP, e.g., 10-100 nm larger.

In the present technology, the surface of the LNPs may also be charged (measured as zeta potential), so long as the net charge is not too great, e.g., −40 mV to +40 mV, preferably from −30 mV to +30 mV. Nanoparticle surface potential may be measured by DLS in an applied electric field at any suitable voltage (e.g., 40 V; the measured surface potential will be independent of the exact voltage used) at 0.1 mg/mL, pH 7.4, 25° C. Examples of the surface potential of the present LNPs include −40, −30, −25, −20, −15, −10, −5, +5, +15, +20, +25, +30, or +40 mV, or a range between and including any two of the foregoing values. Thus, e.g., the surface potential may be, e.g., −20 to +20 mV, −10 to +10, or −5 to +5 mV. In any embodiments, the zeta potential of the LNP filled/complexed with a payload, is closer to neutral than the empty/unfilled/uncomplexed LNP, e.g., about +20 to about +30 mV for LNP filled/complexed to RNP or about +15 mV to about +22 mV for filled/complexed to RNP/ssODN versus about +35 to about +40 for empty/uncomplexed LNP.

The disulfide-containing lipopeptides of the present technology may also be used to improve the efficiency and efficacy of other nanoparticles, including inorganic nanoparticles (e.g., gold nanoparticles², iron oxide nanoparticle²⁴, silica nanoparticles²⁵, and the like), organic nanoparticles (e.g., polymeric nanoparticles made of polyester^{26, 27}, polyamides^{28, 29}, polyanhydrides^{30, 31}, and the like), metal-organic frameworks (e.g., ZIF-8 nanoparticles³² and the like). In some embodiments, a compound of the present technology (e.g., a compound of Formula I) coats an exterior surface of an inorganic nanoparticle, e.g., those found in any of WO 2021/236629, US 2022-0105202, U.S. Pat. No. 10,821,082, WO 2015/075942, US 2018/0185281, the contents of each of which are incorporated by reference in their entirety herein and for all purposes. In any embodiments, the inorganic nanoparticle is a silica nanoparticle (SNP). In any embodiments, the SNP comprises crosslinked polysiloxanes comprising siloxy subunits having one or more of the structures of Formulas IV, IVA, or IVB:

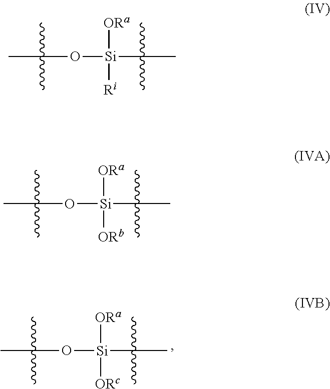

wherein
R¹ at each occurrence is independently selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group, or two R¹ groups, together with the N atom to which they are attached, form a pyrrolidine or piperidine ring;
R^{a} and R^{b} at each occurrence in the polysiloxane are independently selected from a bond to a Si of another polysiloxane chain or C₁₋₆ alkyl groups, and
R^{c} is selected from C₂₋₆ alkenyl groups.

In any embodiments of present technology herein, the SNPs comprise crosslinked polysiloxanes comprising siloxy subunits having the structure of Formula I. In any embodiments the SNPs comprise crosslinked polysiloxanes comprising siloxy subunits having the structure of Formula II. In any embodiments the SNPs comprise crosslinked polysiloxanes comprising siloxy subunits having the structure of Formula III. In any embodiments the SNPs comprise crosslinked polysiloxanes comprising siloxy subunits having the structures of Formulas IV and IVA, or Formulas IVA and IVB, or Formulas IV and IVB, or Formulas IV, IVA, and IVB.

In any embodiments of present technology herein, the crosslinks between polysiloxanes in the SNP comprise disulfide linkages. In any embodiments, the exterior surface of the SNP comprises the silica network. In any embodiments, the exterior surface of the SNP comprises surface-modifying groups attached to and surrounding the silica network, wherein the surface-modifying groups comprise polyethylene glycol (PEG), polysarcosine, polyzwitterion, polycation, polyanion, or combinations of two or more thereof.

In any embodiments, the polysiloxanes of SNPs comprising the plurality of siloxy subunits having the structure (IVA)

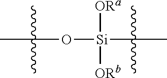

may include a first portion of siloxy subunits wherein R^{a} and R^{b} are independently selected from C₁₋₆ alkyl groups, and a second portion of siloxy subunits wherein one of R^{a} and R^{b} is independently selected from C₁₋₆ alkyl groups at each occurrence, and one of R^{a} and R^{b} is a bond to a Si of another polysiloxane chain. In any embodiments, the polysiloxanes comprising the plurality of siloxy subunits having the structure (IVA), may include a portion of the siloxy subunits wherein each of R^{a} and R^{b} is a bond to a Si of another polysiloxane chain. It will be appreciated that when R^{a} or R^{b} is a bond to a Si of another polysiloxane chain, the siloxysubunit is branched, forming a crosslink to another polysiloxane chain. In any embodiments, the plurality of siloxy subunits may be derived from tetraethoxysilane, i.e., these monomers are precursors which polymerize to form the siloxy subunits.

In any embodiment of any of the SNPs herein, including any siloxy subunits disclosed herein, such as, but not limited to siloxy subunits of structures (IV), (IVA), or (IVB), R^{a} at each occurrence may be independently selected from a bond to a Si of another polysiloxane chain or a C₁₋₆ alkyl group.

In any embodiment of the present SNPs including a siloxy subunit of structure (IV), Rⁱ at each occurrence may independently be selected from the group consisting of C₁₋₁₂ alkyl and C₂₋₁₂ alkenyl groups, optionally substituted with a substituent selected from the group consisting of halogen and NR¹², wherein each occurrence of R¹ is independently selected from H or a C₁₋₁₂ alkyl group.

In any embodiments, Rⁱ at each occurrence may be independently selected from the group consisting of C₁₋₁₂ alkyl and C₂₋₁₂ alkenyl groups, optionally substituted with a substituent selected from the group consisting of halogen and NR¹², wherein the two R¹ groups, together with the N atom to which they are attached, form a pyrrolidine or piperidine ring. In any embodiments, Rⁱ at each occurrence may be a C₁₋₄ alkyl group, optionally substituted with a halogen or NR¹² substituent. In any embodiments, Rⁱ at each occurrence may be a C₂₋₄ alkenyl group. In any embodiments, Rⁱ at each occurrence may be independently selected from methyl, propyl, 3-chloropropyl, 3-aminopropyl, 3-dimethylaminopropyl, and vinyl. In any embodiments, the siloxy subunits of structure (I) may be derived from one or more of triethoxymethylsilane, triethoxypropylsilane, triethoxy-3-chloropropylsilane, triethoxy-3-aminopropylsilane, triethoxy-3-dimethylaminopropylsilane, triethoxyoctylsilane, or triethoxyvinylsilane.

SNPs of the present technology are multifunctional. The SNPs may include weakly basic groups, disulfide linkages, and/or surface-modifying groups. In any embodiments, the weakly basic groups are absent. In any embodiments in which the weakly basic groups are present, they may include heteroaryl groups having a pka of about 4.5 to about 7.2, e.g., about 4.5, about 5, about 5.5, about 6, about 6.3, about 6.5, about 6.7, about 7, about 7.2 or a range between and including any two of the foregoing values. For example, the weakly basic groups may include imidazolyl, pyridinyl, picolinyl, lutidinyl, indolinyl, tetrahydroquinolinyl, or quinolinyl groups or a combination of two or more of the foregoing groups. In any embodiments, the weakly basic groups may include an imidazolyl group and/or pyridinyl group. In any embodiments, each weakly basic group is attached to a siloxy subunit and includes one of the following Formulas A, B, or C:

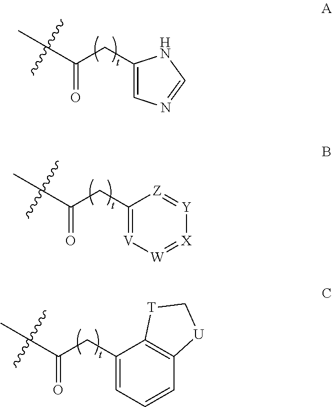

wherein
t at each occurrence is independently 0, 1, 2 or 3
one of T and U is NH and the other is CH₂;
one of V, W, X, Y, Z is N and the rest are selected from CH or CCH₃.

In any embodiments, the polysiloxanes may include siloxy subunits having the structures (IVC) or (IVD),

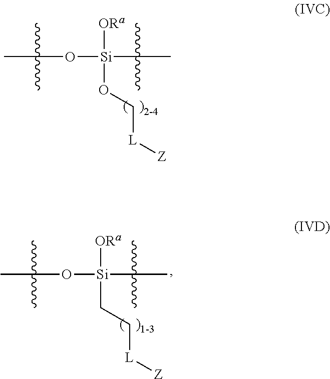

wherein
R^{a} at each occurrence is independently selected from C₁₋₆ alkyl groups or a bond to a Si of another polysiloxane chain;
L is a bond or is a linking group selected from —C(O)NH—, —O—, —NH—, —C(O)—, or —C(O)O; and
Z is at each occurrence independently a picolinyl, lutidinyl, indolinyl, tetrahydroquinolinyl, quinolinyl, imidazolyl, or pyridinyl group.

In any embodiments, the polysiloxanes may include siloxy subunits having the structure (IVD). In any embodiments, L may be —C(O)NH—. In any embodiments, Z may be imidazolyl. In any embodiments, the weakly basic groups may, e.g., comprise a siloxy subunit derived from N-(3-(triethoxysilyl)propyl)-1H-imidazole-2-carboxamide (TESPIC).

In any embodiments of the SNPs herein, the polysiloxanes may further include silyloxy subunits having structure (IVE):

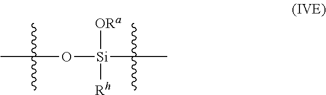

wherein
R^{a} at each occurrence is independently selected from a bond to a Si of another polysiloxane chain or a C₁₋₁₂ alkyl group; and
R^{h} at each occurrence is a moiety comprising a weakly basic group.

In any embodiments, the moiety of R^{h} may have a molecular weight of up to 300 Da and comprise any of the weakly basic groups disclosed herein. For example, the weakly basic group of R^{h} may be selected from imidazolyl, pyridinyl, tetrahydroquinolinyl, or indolinyl groups, or a combination any two or more thereof. In any embodiments, R^{h} at each occurrence may independently have the structure —(CH₂)ₙ-L-Z, wherein L is a bond or is a linking group selected from —C(O)NH—, —O—, —NH—, —C(O)—, or —C(O)O; Z is at each occurrence is independently a picolinyl, lutidinyl, indolinyl, tetrahydroquinolinyl, quinolinyl, imidazolyl, or pyridinyl group; and n is 0, 1, 2, 3, or 4. In any embodiments, R^{h} may have the structure (D):

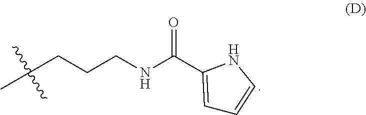 [SMILES: *CCCNC(=O)c1ccc[nH]1]

The polysiloxanes that make up the silica network are crosslinked, not only by siloxy linkages, but including by disulfide linkages. For example, the polysiloxanes may include a plurality of crosslinking siloxy subunits having the structure (V)

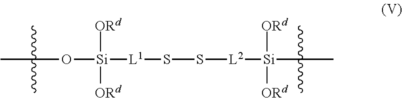

wherein L¹ and L² at each occurrence in the polysiloxanes are independently a C₁₋₆ alkylene group; R^{d} at each occurrence in the polysiloxanes is the same or different and is independently selected from a bond to another polysiloxane chain or C₁₋₆ alkyl groups. In any embodiments R^{d} may be the same at each occurrence, e.g., ethyl. In any embodiments, each of L¹ and L² may be ethylene, propylene, or butylene at each occurrence. In any embodiments, each of L¹ and L² may be propylene, at each occurrence. The disulfide bonds are sensitive to the levels of glutathione (GSH) naturally found in cells. While not wishing to be bound by theory, when SNPs enter a cell, the GSH in the cell is believed to reduce the disulfide bonds in the silica network, causing the silica network to fall apart and release any encapsulated or associated biomolecule (e.g., biomacromolecule) into the cytosol of the cell.

SNPs of the present technology include an exterior surface comprising surface-modifying groups attached to and surrounding the silica network. The surface-modifying groups may comprise polyethylene glycol (PEG), polysarcosine, polyzwitterion, polycation, polyanion, or combinations of two or more thereof. In any embodiments, the surface-modifying groups may comprise polyethylene glycol (PEG), polysarcosine, polyzwitterion or combinations of two or more of thereof, or PEG, polysarcosine, polycation, polyanion, polyzwitterion or combinations of two or more of thereof. In any embodiments, the surface-modifying groups may include PEG and/or polysarcosine. The surface-modifying groups may further be conjugated to one or more of targeting ligands, biotin, CPP, imaging agents, or dyes.

PEG is a hydrophilic polymer comprising repeating ethylene oxide subunits and may be used as a surface-modifying group of the present SNPs. The PEG polymeric chains may be attached directly or through a linker to the polysiloxanes of the silica network. Each PEG terminates in one of various groups that, e.g., may be selected from a targeting ligand, OH, O—(C₁₋₆)alkyl, NH₂, CPP, biotin or a dye. In some embodiments the PEG terminates in OH or O—(C₁₋₆)alkyl, and in still others the PEG terminates in in an OC₁₋₃ alkyl group. In still other embodiments, the PEG terminates in a targeting ligand. The targeting ligand may be selected from the group consisting of a cofactor, carbohydrate, peptide, antibody, nanobody, or aptamer. For example, the targeting ligand maybe selected from the group consisting of glucose, RVG peptide, folic acid, mannose, GE11, cRGD, KE108, octreotide, PSMA aptamer, TRC105, 7D12 nanobody, all-trans retinoic acid (ATRA), 11-cis-retinal (11cRal), CTB, N-acetylgalactosamine (GalNAc) and combinations of two or more thereof. In other embodiments, the targeting ligand is selected from the group consisting of folic acid, mannose, GE11, cRGD, KE108, octreotide, TAT cell penetrating peptide, PSMA aptamer, TRC105, 7D12 nanobody, all-trans retinoic acid (ATRA), 11-cis-retinal (11cRal), CTB, and N-acetylgalactosamine (GalNAc). In any embodiments, the targeting ligand is selected from glucose, RVG peptide, or both.

In any embodiments of the SNP, the polysiloxanes may comprise a plurality of siloxy subunits having the structure (VI):

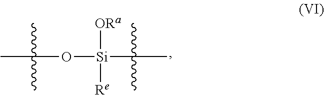

wherein R^{a} at each occurrence is selected from a bond to Si from another polysiloxane chain or a C₁₋₆ alkyl group, and R^{e} at each occurrence is surface-modifying group, optionally including a C₁₋₆ linker group connecting the surface-modifying group to the Si atom to which R^{e} is attached. In certain embodiments, the C₁₋₆ linker group is present and connected to the surface-modifying group directly or via an amine, ether, amide, ester, urethane, urea, imine, or sulfide group. For example, the C₁-6 linker group may be —NHC(O)NH—(C₂₋₅ alkylene)-, —NHC(O)—(C₂₋₅ alkylene)-, —C(O)NH—(C₂₋₅ alkylene)-, —NH—(C₂₋₅ alkylene)-, —O—(C₂₋₅ alkylene)-, —S—(C₂₋₅ alkylene)-, —OC(O)NH—(C₂₋₅ alkylene)-, or —NHC(O)O—(C₂₋₅ alkylene)-. In any embodiments, the surface-modifying groups may comprise PEG attached to a siloxy subunit having the structure (VII):

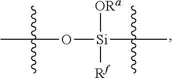

wherein R^{a} at each occurrence is selected from a bond to Si from another polysiloxane chain or a C₁₋₆ alkyl group, and R^{f} has the structure (E1) or (E2):

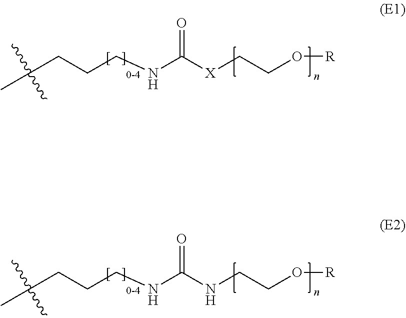

wherein X is O, NH, or CH₂O, and R is selected from the group consisting of H, a C₁₋₆ alkyl, targeting ligand, a cell-penetrating peptide (CPP), and an imaging agent. In any embodiments, the silica network may comprise two or more (e.g., 2, 3, 4, or 5) different siloxy subunits of structure (VII). For example in some embodiments, the silica network comprises siloxy subunits of structure (VII) wherein X is NH and R is a C₁₋₆ alkyl and siloxy subunits of structure (VII) wherein X is NH and R is a targeting ligand. In any embodiments, two or more distinct targeting ligands (e.g., glucose and RVG peptide) may be used (i.e., on two different siloxy subunits of structure (VII). In any embodiments, the surface-modifying groups may comprise PEG attached to a siloxy subunit having the structure, —O—Si(R^{g})₂—, wherein R^{g} at each occurrence is independently selected from OR^{a} or R^{f} as defined herein.

In the present technology, the surface of the SNPs may be charged due to the presence of surface-modifying groups that include ionizable functional groups on the SNP surface and/or in the SNP surface layer, provided the net charge is as described herein. For example, in any embodiments, the polysiloxanes of the silica network may comprise a plurality of siloxy subunits having the structure (VIII):

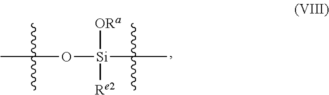

wherein R^{a} at each occurrence in the polysiloxane is a bond to Si from another polysiloxane chain or a C₁₋₆ alkyl group, and R^{e2} at each occurrence is a C₁₋₁₂ alkyl group, e.g., a C₁₋₆ alkyl group, substituted with a charged functional group. The charged functional groups may include positively and/or negatively charged functional groups, or ionizable functional groups that provide positively and/or negatively charged groups.

In any embodiments, the surface-modifying groups may include positively charged functional groups. In any embodiments, the positively charged functional groups may include an ionizable group selected from amine, amidine, guanidine, pyridinyl or combinations of two or more thereof. For example, R^{e2} may be an amino-(C₂₋₄ alkyl) group such as an amino propyl group. The surface-modifying groups may also include a cationic polymer or CPP. For example, the cationic polymer may be selected from the group consisting of polyethyleneimine (PEI), polylysine, polyarginine, and polyamidoamine (PAMAM). In any embodiments, the CPP may be selected from any of those disclosed herein.

In any embodiments, the surface-modifying groups may include negatively charged groups. In any embodiments, the negatively charged groups may include ionizable functional groups selected from carboxyl, sulfonyl, sulfate, phosphate, or combinations thereof. In any embodiments, R^{o} may be a carboxyl-(C₂₋₄ alkyl) group. The surface-modifying groups may also include an anionic polymer. In any embodiments, the anionic polymer may be selected from the group consisting of poly(glutamic acid) and poly(acrylic acid).

In any embodiments, the surface-modifying groups may include positively charged functional groups and negatively charged groups, i.e., a polyzwitterion. The polyzwitterion may include any combination of the positively and negatively charged groups disclosed herein. In any embodiments, the surface-modifying group may be a polyzwitterion selected from poly(carboxybetaine methacrylate) (PCBMA), poly(sulfobetaine methacrylate) (PSBMA), poly(2-methacryloyloxyethyl phosphorylcholine) (PMPC), and combinations of two or more thereof.

In any embodiments of any of the aspects of the present technology (including but not limited to the first, second or third aspects), the silica network of the SNPs comprises siloxy subunits having structure (I). In some such embodiments, the siloxy subunits having structure (IV) comprise 1 to 80 mol % of the silica network, including for example, 1 mol %, 2 mol %, 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, or 80 mol % or a range between and including any two of the foregoing values. Additional examples of the present SNPs may therefore include from 10 mol % to 70 mol % or from 20 mol % to 60 mol %. In some embodiments of the first, second or third aspects of the present technology, no siloxy subunits of structures (II) or (III) are included in the silica network of the SNPs.

In any embodiments of any of the aspects of the present SNPs, the siloxy subunits having structures (IVA) or (IVB) comprise 1 to 80 mol % of the silica network, including for example, 1 mol %, 2 mol %, 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, or 80 mol % or a range between and including any two of the foregoing values.

In any embodiments of any of the aspects of the present SNPs, the molar percentage of disulfide-containing crosslinker (e.g., having the structure (V)) to the total siloxy subunits may range from 20 mol % to 99 mol %, including for example, 20 mol %, 30 mol %, 40 mol %, 50 mol %, 60 mol %, 70 mol %, 80 mol %, 85%, 90%, 95%, 96%, 97%, 98%, 99%, or a range between and including any two of the foregoing values, e.g., 20 mol % to 80 mol % or 20 mol % to 90 mol %. The molar ratio of siloxy subunits bearing weakly basic groups (e.g., having any of structures (IVA), (IVB), or (IVC)) to total siloxy subunits of the silica network may range from 0 mol % to 40 mol %, e.g., 0 mol %, 5 mol %, 10 mol %, 15 mol %, 20 mol %, 30 mol %, 40 mol % or a range between and including any two of the foregoing values. The molar ratio of siloxy subunits bearing surface-modifying groups (e.g., having structure IVA or IVB) to the total organosilica precursors may range from 10 mol % to 50 mol %, e.g., 10 mol %, 15 mol %, 20 mol %, 30 mol %, 40 mol %, or 50 mol % or a range between and including any two of the foregoing values.

In the present technology, the surface of the SNPs may also be charged (measured as zeta potential), so long as the net charge is not too great, e.g., −45 mV to +45 mV, preferably from −30 mV to +30 mV. Nanoparticle surface potential may be measured by DLS in an applied electric field at any suitable voltage (e.g., 40 V; the measured surface potential will be independent of the exact voltage used) at 0.1 mg/mL, pH 7.4, 25° C. Examples of the surface potential of the present SNPs include −45, −30, −25, −20, −15, −10, −5, +5, +15, +20, +25, +30, or +45 mV, or a range between and including any two of the foregoing values. Thus, e.g., the surface potential may be, e.g., −20 to +20 mV, −10 to +10, or −5 to +5 mV. In any embodiments, where the surface of the SNP bears charged functional groups, the net charge is or is about 0 mV, e.g., due to a polyzwitterion with an equal number of positively and negatively charged groups.

In another aspect, the present technology provides methods of delivering a payload to a target cell, e.g., a mammalian cell, for any suitable purpose, e.g., gene editing, gene silencing, therapy, etc. The methods include exposing the mammalian cell to an effective amount of any of the herein-described LNP. By an effective amount is meant an amount sufficient to produce a detectable or measurable amount of infiltration of the LNP into the target cell and/or produce a detectable or measurable effect in said cell. In any embodiments of the methods, the mammalian cell may be selected from a kidney cell, fibroblast cell, monocyte/macrophage cell, or melanoma cell. The methods include both in vitro and in vivo methods. For example, the methods may include exposing an effective amount of any of the herein-described LNP to tissue culture. In any embodiments, the cell may be exposed to the LNP via any rout of administration described herein. In any embodiments, the payload is any of those described herein, including but not limited to RNP, plasmid DNA (pDNA), single-stranded donor oligonucleotide (ssODN), antisense oligonucleotides (ASO), NAD⁺, NADH, complementary DNA (cDNA), messenger RNA (mRNA), small interfering RNA (siRNA), microRNA (miRNA), short hairpin RNA (shRNA), guide RNA (gRNA) (including, e.g., trans-activating CRISPR RNA (tracrRNA)/CRISPR RNA (crRNA) complex, tracrRNA, crRNA, single guide RNA (sgRNA)), transfer RNA (tRNA), ribozymes, and combinations of two or more thereof. In any embodiments, the payload is Cas9 RNP or Cas9 RNP+ssODN. In any embodiments, the payload is a Cas9 RNP, Cas12 RNP, Cas13 RNP, Cas14 RNP, or CasΦ RNP, each optionally in combination with an ssODN. In any embodiments, the payload is Cas9 RNP or Cas9 RNP+ssODN. In any embodiments, the payload is mRNA for a Cas polypeptide and a gRNA (as disclosed herein). In any embodiments, the payload is Cas9 mRNA, Cas12 mRNA, Cas13 mRNA, Cas14 mRNA, or CasΦ mRNA, each optionally in combination with an sgRNA, or the payload is a combination of any two or more thereof.

In another aspect, the present technology provides methods of treating a condition or disorder in a subject that may be ameliorated by any of the types of payloads disclosed herein. In any embodiments, the methods include administering to the subject an effective amount of a LNP as as disclosed herein, i.e., a therapeutically effective amount to ameliorate or cure the condition or disorder. For example, the methods may include administering any of the herein-described LNPs to a subject in need thereof (i.e., a subject in need of the biomolecule (e.g., biomacromolecule) to be delivered by the nanoparticle). As used herein, a “subject” is a mammal, such as a cat, dog, rodent, cow, pig or primate. In some embodiments, the subject is a human. In some embodiments, the payload is any of those described herein, including but not limited to RNP, pDNA, ssODN, ASO, NAD⁺, NADH cDNA, mRNA, siRNA, miRNA, shRNA, gRNA (including, e.g., tracrRNA, crRNA, tracrRNA/crRNA complex, and sgRNA), tRNA, ribozymes, and combinations of two or more thereof. In any embodiments, the payload is a Cas9 RNP, Cas12 RNP, Cas13 RNP, Cas14 RNP, or CasΦ RNP, each optionally in combination with an ssODN, In any embodiments, the payload is Cas9 RNP or Cas9 RNP+ssODN. In any embodiments, the payload is mRNA for a Cas polypeptide and a gRNA (as disclosed herein). In any embodiments, the payload is Cas9 mRNA, Cas12 mRNA, Cas13 mRNA, Cas14 mRNA, or CasΦ mRNA, each optionally in combination with a gRNA (as disclosed herein), or the payload is a combination of any two or more thereof.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not degrade the LNPs described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol),

Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present LNPs (loaded with a biomolecule) to the patient and may include 0.1, 0.2, 0.3, 0.4, 0.5, 0.75, 1.0, 1.5, 2.0, 2.5, 3.0, 4.0, 5.0, 6.0, 7.0, 8.0, 9.0, 10, 11, 12, 13, 14, 15 mg/kg or a range between and including any two of the forgoing values such as 0.1 to 15 mg/kg. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### Materials and General Procedures

Materials and Instrumentation. Cystamine dihydrochloride, L-lysine methyl ester dihydrochloride (H-Lys-OMe·2HCl), Nα-Boc-Nω-(2,2,4,6,7-pentamethyl-dihydrobenzo furan-5-sulfonyl)-L-arginine (Boc-Arg(Pbf)-OH), N,N-diisopropylethylamine (DIEA), triethylamine (TEA), Cholesterol and Di-tert-butyl dicarbonate (Boc anhydride) were purchased from Chem Impex International; Boc-Arg(Pbf)-OH also purchased from Alfa Aesar. H-Arg(Pbf)-OMe hydrochloride and Boc-cystamine were purchased from Asta Tech. Tetradecyl acrylate and stearyl acrylate were purchased from TCI America. Acryloyl chloride, boc-hexanediamine, sucrose, sodium chloride (NaCl), sodium hydroxide (NaOH), hydrochloric acid (HCl, 37% in H₂O), sodium bicarbonate (NaHCO₃), dimethylformamide (DMF), and trifluoroacetic acid (TFA) were purchased from Sigma Aldrich. 1-Ethyl-3-(3-dimethylaminopropyl) carbodiimide (EDC), 1-hydroxybenzotriazole hydrate (HOBT), hexafluorophosphate benzotriazole tetramethyl uronium (HBTU), and magnesium sulfate (MgSO₄) were purchased from Oakwood Chemical. Oleic acid was purchased from TCI America. Oleyl alcohol, methanol (MeOH), ethanol (EtOH), dichloromethane (DCM), ethyl acetate, hexane, tetrahydrofuran (THF), chloroform, and solvents for purification were purchased from Fisher Chemical, and ultrapure water was supplied by Milli-Q system (MilliporeSigma). 1,2-Distearoyl-sn-glycero-3-phosphoethanolamine-N-[carbonyl-methoxy(polyethylene glycol)-2000] (DSPE-PEG2000), 1,2-dioleoyl-sn-glycero-3-phospho-L-serine (DOPS), and 1,2-distearoyl-sn-glycero-3-phosphocholine (DSPC) were purchased from Avanti Polar Lipids. 1,2-dimyristoyl-rac-glycero-3-methoxypolyethylene glycol-2000 (DMG-PEG2k) and 24α-Ethyl cholesterol were purchased from Cayman Chemical. Slide-A-Lyzer Mini Dialysis Units, Molecular weight cutoff (MWCO) 20 kDa, were purchased from Fisher Scientific. sNLS-Cas9-sNLS nuclease was purchased from Aldevron. The sgRNAs, ssODN and all the primers for sequencing and RT-qPCR were customized from Integrated DNA Technologies. mCherry-mRNA, GFP mRNA, Cas9 mRNA, and Cre mRNA were purchased from TriLink BioTechnologies. 1M Citrate buffer (pH 3), 1M Tris-HCl buffer (pH 7.5), cell culture media and supplements were purchased from Thermo Fisher Scientific. The cell viability assay reagent, 3-(4,5-dimethylthiazol-2-yl)-2,5-diphenyltetrazolium bromide (MTT) was purchased from Thermo Fisher Scientific. All antibodies for immunostaining and Masson's Trichrome Stain Kit were purchased from Abcam. Hematoxylin and eosin reagents were purchased from VWR. Q5 High-Fidelity 2× Master Mix (#M0492) and PCR & DNA Cleanup Kit (#T1030) were purchased from New England Biolabs. RNA extraction kit was purchased from Thermo Fisher Scientific. All the reagents for RT-qPCR were purchased from Bio-Rad Laboratories. Nuclear magnetic resonance (NMR) spectroscopy was performed on an Avance 400 (Bruker Corporation, USA).

Cell culture. HEK 293 cells and GFP-HEK 293 cells were cultured in a 6-well culture plate (Falcon) with the Dulbecco's Modified Eagle Medium supplemented with 10% (v/v) fetal bovine serum (Thermo Fisher Scientific) and 1% (v/v) Penicillin-Streptomycin (Thermo Fisher Scientific). Cells were kept in an incubator (Thermo Fisher Scientific) at 37° C. with 5% carbon dioxide atmosphere at 100% humidity. NIH 3T3 cells (a mouse embryonic fibroblast cell line) were purchased from ATCC (USA) and cultured with DMEM medium (Gibco, USA) with 10% (v/v) fetal bovine serum (FBS, Gibco, USA) and 1% (v/v) penicillin-streptomycin (Gibco, USA)

In vitro gene editing study. GFP-HEK 293 cells were seeded in a 96-well culture plate (Falcon) at the density of 5,000 cells per well 24 h before treatment. Cas9 nuclease was complexed with GFP-sgRNA (Table 1, molar ratio of Cas9:sgRNA=1:1) designed for GFP knock-down to form RNP. The RNP solution was mixed with different amounts of Empty-LNP to form RNP-LNP with various LNP and RNP weight ratios. The solutions were further diluted by PBS and the final RNP concentration in each formulation was 30 ng/L. The final solution (10 μL) was added to each well. The dose of RNP was kept as 300 ng/well. Commercially available transfection reagent, Lipofectamine 2000 was used as a control, and payload-Lipofectamine 2000 complexes were prepared following manufacturer's guidance. 0.5 μL Lipofectamine 2000 (Thermo Fisher Scientific) and 300 ng RNP were each diluted to 5 μL with Opti-MEM medium (Thermo Fisher Scientific), respectively. Then, these two solutions were combined and incubated for 5 min. This L solution was then added to each well. To study the GSH responsive behavior, the normal culture medium was replaced the culture medium with a GSH concentration ranging from 0 to 10 mM before adding the RNP-LNP. Cells were harvested and resuspended in 500 μL PBS 96 hours post-treatment. The editing efficiency was analyzed by flow cytometry by quantifying the ratio of GFP-negative cells (Attune NxT, Thermo Fisher Scientific) with FlowJo.

TABLE 1
Protospacers of sgRNAs (5′ to 3′).

| | |
|---|---|
| GFP | GCACGGGCAGCTTGCCGG |
| SV40 PolyA (Ai14) | AAGTAAAACCTCTACAAATG |
| DMD | TCTTTGAAAGAGCAATAAAA |

Gel electrophoresis study. Gels were cast using 2% w/v agarose solution in tris-acetate-ethylenediaminetetraacetic acid buffer containing SYBR Safe Green Gel Stain. RNP-LNPs with different weight ratios were prepared as previously described and were loaded with an equivalent dosage of 3 μg RNP per well in the gel. Electrophoresis was conducted with a voltage at 110 V for 30 minutes.

Intracellular trafficking study. 1 μL Atto550 tracrRNA (200 μM) was mixed with 1 μL crRNA (200 μM) and incubated at room temperature for 5 minutes. 3.2 μL Cas9 protein (10 mg/mL) was added to the above mixture and incubated for another 5 minutes to form ATTO-labeled RNP. The RNP-LNP was formed with the same method described previously. HEK 293 cells were treated with RNP-LNP at a dose of 300 ng/well. At certain time points (i.e., 2, 4 and 6 h), cells were washed with 1×DPBS three times and treated with Hoechst 33342 (10 μg/mL) for 10 minutes to stain the nuclei. Subsequently, cells were washed with 1×DPBS three times and treated with LysoTracker Green DND-26 (10 μM) to stain endosomes/lysosomes. Cells were imaged using an AR1 confocal microscope.

In vitro DNA plasmid and mRNA transfection study. HEK 293, NIH 3T3, RAW 264.7 or B78 cells were seeded in a 96-well culture plate (Falcon) at the density of 10,000 cells per well 24 h before treatment. Empty-LNP solution was mixed with RFP-plasmid or RFP-mRNA at different weight ratios by using the same preparation procedures for RNP-LNP. The dose of RFP-plasmid or RFP-mRNA was fixed at 200 ng/well. For lipofection, 0.5 μL Lipofectamine 2000 and 200 ng FRP-plasmid or RFP-mRNA were used for each well. The preparation method was similar as described above. After 48 hours, the cells were harvested with 0.25% trypsin-EDTA (Gibco, USA) and resuspended with 300 μL of DMEM. The mRNA transfection efficiencies were obtained with a flow cytometer and analyzed with Flow Jo 7.6. For in vitro transfection of the additional lipopeptide formulations, NIH 3T3 cells were transfected with GFP-mRNA-loaded LNPs with a mRNA dosage of 200 ng/well. For lipofection, 0.5 μL Lipofectamine 2000 and 200 ng GFP-mRNA were used for each well. Transfection efficiency was analyzed 48 h post-treatment as described above. For in vitro transfection of mRNA-loaded SNP with lipopepetide coating, NIH 3T3 cells were transfected with GFP-mRNA-loaded SNP (prepared as described in WO2021/236629) with or without lipopeptide coating at an mRNA dosage of 200 ng/well. An untreated group was used as the negative control group. Transfection efficiency was analyzed 48 h post-treatment as described above.

Cell viability assay. HEK 293 cells were seeded in a 96-well culture plate at the density of 10,000 cells/well 24 h before treatment. Cells were treated with 200 ng per well of DNA, which was complexed with either Lipofectamine 2000 (5 mg/L) or LNP with a concentration ranging from 5 mg/L to 20 mg/L. 48 h after treatment, the media was removed and media containing 500 μg/mL MTT (Thermo Fisher, USA) was added and incubated for 4 h. Then the media was aspirated carefully, and the purple precipitate was dissolved in 150 μL of DMSO. The absorbance at 560 nm was measured with a microplate reader (GloMax® Multi Detection System, Promega, USA). The cell viability was calculated by comparing the absorbance of treatment groups with the control groups.

Animals. All animal experiments were approved by the UW-Madison Institutional Animal Care and Use Committee. All the mice were obtained from The Jackson Laboratory. B6.Cg-Gt(ROSA)26Sor^{tm14(CAG-tdTomato)Hze}/J mice (Ai14 mice, #007914) of 4 weeks old were used to test in vivo gene editing efficiency of RNP-LNP. Male C57BL/10ScSn-Dm^{dmx}/J mice (mdx mice, #001801) were specially fed (LabDiet® 5K52) and used to test RNP/ssODN-LNP targeting the DMD gene. C57BL/6J mice (#000664) were used as a positive control without treatment.

In vivo gene editing in Ai14 mice. Cas9 nuclease was complexed with Ai14-sgRNA (Table 1, molar ratio of Cas9:sgRNA=1:1) designed for the excision of SV40 polyA blocks to form RNP. RNP-LNP (w/w=4:1) was prepared as described above. 40 μL RNP-LNP (RNP concentration 0.6 μg/L) and 40 μL PBS was intramuscularly injected into the tibialis anterior muscle of Ai14 mice (n=3) on the left leg and right leg, respectively. After 14 days, mice were euthanized, and tibialis anterior muscles were isolated and imaged by IVIS. Thereafter the muscles were saved at −80° C. for further analysis.

In vivo gene editing in mdx mice. Cas9 nuclease was complexed with DMD-sgRNA targeting exon 23 in the DMD gene (Table 1) and further mixed with ssODN (Table 2, molar ratio of Cas9:sgRNA:ssODN=1:1:1). RNP/ssODN-LNP was prepared as described above. Mdx mice were intramuscularly injected with PBS (negative control, n=8) or RNP/ssODN-LNP (containing 0.6 μg/L RNP and 0.18 μg/L ssODN, n=9) in tibialis anterior muscles (20 μL), gastrocnemius muscles (20 μL) and triceps brachii muscles (40 μL). C57BL/6 wild type mice (n=5) without injection were used as the positive control. The hanging time of the mice was tested weekly to monitor their muscle strength. After 35 days, mice were euthanized, and the injected muscles were isolated. Muscles were collected either for section or frozen by liquid nitrogen and stored at −80° C. for further analysis.

TABLE 2
Sequences of ssODN (5′ to 3′).

| | |
|---|---|
| DMD | TGATATGAATGAAACTCATCAAATATGCGTGTTAGTGT |
| | AAATGAACTTCTATTTAATTTTGAGGCTCTGCAAAGTTC |
| | TTTAAAGGAGCAGCAGAATGGCTTCAACTATCTGAGTG |
| | ACACTGTGAAGGAGATGGCCAAGAAAGCACCTTCAGAA |
| | ATATGCCAGAAATATCTGTCAGAATTT |

Hanging time assay. Three weeks after injection, four-limb hanging time assays were performed weekly for RNP/ssODN-LNP or PBS-treated mdx mice and wild-type mice. In brief, a grid was placed 25 cm above a cage to challenge mice and soft bedding was placed at the bottom for protection. The time of a mouse hanging on the grid before falling was recorded. The test ended when a mouse can hang over 600 s and the hanging time was recorded as 600 s. Tests were repeated 3 times for representative hanging time.

Ex vivo imaging assay. tdTomato expression level in tibialis anterior muscles of Ai14 mice was measured by in vivo imaging system (IVIS) by using the excitation wavelength of 554 nm and emission wavelength at 581 nm.

Muscle tissue section. The muscles were fixed in 4% paraformaldehyde (PFA) at 4° C. for 24 h, then switched to PBS solution containing 30% sucrose at 4° C. for 72 h. Thereafter, the tissues were embedded in Tissue-Tek® Optimal Cutting Temperature (OCT) Compound (Sakura Finetek, USA), and frozen in dry ice. The blocks were sectioned using a cryostat machine (CM1900, Leica Biosystems, USA) with the thickness of 5 m and mounted on microscope slides.

Immunostaining of muscle sections. For Immunostaining of tdTomato, the slices were first washed with PBS to remove OCT and incubated in a blocking solution (10% goat serum and 0.3% Triton X-100 in PBS) at room temperature for 1 h. The slices were then incubated with a rabbit anti-RFP primary antibody (ab152123, 1:1000, Abcam, USA) for 1 h followed by incubation with a secondary antibody (ab150080, 1:1000, Abcam, USA) for another hour at room temperature. The slides were washed with PBS for three times and were subsequently stained with DAPI for 5 minutes at room temperature followed by washing in PBS for three times. Thereafter, the slides were covered with microscope cover glasses. For immunostaining of dystrophin, the muscle sections went through the same procedures by using a dystrophin primary antibody (sc-47760, 1:100, Santa Cruz Biotechnology) and a secondary antibody (ab150080, 1:1000, Abcam, USA).

Masson' staining. Masson Staining was conducted by using a Trichrome Stain Kit (ab150686, Abcam, USA) following the manufacturer's instruction. Muscle sections were washed with PBS to remove OCT and re-fixed in Bouin's solution at 60° C. for 1 h and washed with running tap water. The slices were incubated in the mixture of Hematoxylin solution A&B for 5 min and washed with running tap water. Then the slices were stained with Biebrich Scarlet-Acid Fuchsin Solution for 5 min and differentiated by phosphotungstic phosphomolybdic acid for 10 min following incubation with Aniline Blue for up to 15 min and washing with DI water. Thereafter slices were dehydrated by 95% ethanol, 100% ethanol, and 100% Xylene (5 s for each) and finally mounted with optical resin glue and observed under an optical microscope.

H&E staining. H&E staining was performed by using the hematoxylin and eosin reagents following the manufacture's instruction. Muscle sections were washed with PBS to remove OCT and incubated in hematoxylin solution (#3801579, VWR) for 2 min and then differentiated in 1% hydrochloric alcohol solution for 10-15 s to stain the nuclei. Then, the slices were further incubated with eosin solution (#3801619, VWR) for 30 s to stain the cell cytosol. Washing with DI water was applied after each step. Slices were dehydrated in 100% alcohol for 10 min twice and incubated in 100% Xylene for 10 min twice and finally mounted by optical resin glue and observed under an optical microscope.

Sanger sequencing. To prepare the samples for Sanger sequencing, genomic DNA was extracted, amplified, and purified with commercially available kits following the manufacturers' protocols. Briefly, genomic DNA extraction was performed on skeletal muscles from RNP/ssODN-LNP-treated and PBS-treated mdx mice with Monarch® Genomic DNA Purification Kit (#T3010, New England Biolabs). The DNA of the interested region was amplified by PCR with designed primers (Table 3) and Q5® High-Fidelity 2× Master Mix (#M0492, New England Biolabs). The PCR products were purified with Monarch@ PCR & DNA Cleanup Kit (#T1030, New England Biolabs). For each sample, 20 ng of the above PCR product was mixed with 25 pmol forward primer (Table 3) and diluted to 15 μL by ultrapure water. The samples were sequenced by GENEWIZ and analyzed by SnapGene Viewer v5.3 and TIDE web tool (http://shinyapps.datacurators.nl/tide/).

TABLE 3
Sequences of primers (5′ to 3′) for PCR an
RT-qPCR.

| | |
|---|---|
| DMD | Forward: |
| | GAGAAACTTCTGTGATGTGAGGACATATAAAG |
| | Reverse: |
| | CAATATCTTTGAAGGACTCTGGGTAAAATATC |
| GAPDH | Forward: TGAGGCCGGTGCTGAGTATGTCG |
| | Reverse: CCACAGTCTTCTGGGTGGCAGTG |
| IFN-γ | Forward: CAGCAACAGCAAGGCGAAAAAGG |
| | Reverse: TTTCCGCTTCCTGAGGCTGGAT |
| IL-1α | Forward: CGAAGACTACAGTTCTGCCATT |
| | Reverse: GACGTTTCAGAGGTTCTCAGAG |
| IL-1 β | Forward: TGGACCTTCCAGGATGAGGACA |
| | Reverse: GTTCATCTCGGAGCCTGTAGTG |
| IL-6 | Forward: CTGCAAGAGACTTCCATCCAG |
| | Reverse: AGTGGTATAGACAGGTCTGTTGG |
| IL-9 | Forward: TCCACCGTCAAAATGCAGCTGC |
| | Reverse: CCGATGGAAAACAGGCAAGAGTC |
| IL-10 | Forward: CGGGAAGACAATAACTGCACCC |
| | Reverse: CGGTTAGCAGTATGTTGTCCAGC |
| TNF-α | Forward: GGTGCCTATGTCTCAGCCTCTT |
| | Reverse: GCCATAGAACTGATGAGAGGGAG |
| NF-κB | Forward: CTGGCAGCTCTTCTCAAAGC |
| | Reverse: TCCAGGTCATAGAGAGGCTCA |

In vivo immunogenicity assay. The skeletal muscles from RNP/ssODN-LNP or PBS-treated mdx mice were stored in RNAlater solution (Thermo Fisher Scientific) at −20° C. before RNA extraction. The muscles were lysed and homogenized in TRIzol™ Reagent (Thermo Fisher Scientific) and phase separation was conducted by adding chloroform to the mixtures. RNA in the aqueous phase was precipitated with isopropanol, washed with 75% ethanol at 4° C., and resuspended in ultrapure water following the manufacturer's protocol. cDNA was synthesized using iScript™ Reverse Transcription Supermix via PCR (Bio-Rad Laboratories, Inc.) following the manufacturer's protocol. Quantitative RT-PCR (CFX96, Bio-Rad Laboratories, Inc.) was performed with 10 μL samples containing 5 L Taq Universal SYBR Green Supermix (Bio-Rad Laboratories, Inc.), 10 ng cDNA templates, and 0.5 μM customized primers (Table 3)

Statistical Analysis. Data were presented as the mean±standard deviation (SD). Comparisons among groups were analyzed by the t-test or one-way ANOVA with Tukey's post hoc test. The value of P<0.05 was considered as significantly different (*P<0.05, **P<0.01, ***P<0.001, ****P<0.0001).

### Example 1—Preparation of Lipopeptides with Disulfide Linkages

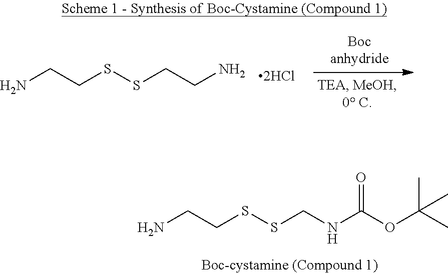

Synthesis of Boc-cystamine (Compound 1). Cystamine dihydrochloride (1.03 g, 4.4 mmol) was added to a round bottom flask and dissolved in 20 mL MeOH. TEA (2.2 mL, 16 mmol) was added to the above solution under stirring and the mixture was cooled down to 0° C. in an ice bath. Boc anhydride (1.02 g, 4.4 mmol) dissolved in 10 mL MeOH was added dropwise to the flask and the reaction was proceeded at 0° C. for 45 minutes under stirring. The solvent was removed by rotary evaporation. The product was dissolved in 30 mL DCM and washed with saturated NaHCO₃ solution. The organic layer was collected and dried by anhydrous MgSO₄ overnight, filtered, and concentrated by rotary evaporation. The product was further purified by column chromatography (MeOH/DCM=1:15) on silica gel to obtain Compound 1. ¹H NMR (400 MHz, CDCl₃): δ 1.45 (s, 9H, Boc group); δ 2.77 (t, 4H, NH₂CH₂CH₂SSCH₂CH₂NH—); δ 3.02 (t, 2H, NH₂CH₂CH₂SSCH₂CH₂NH—); δ 3.45 (t, 2H, NH₂CH₂CH₂SSCH₂CH₂NH—).

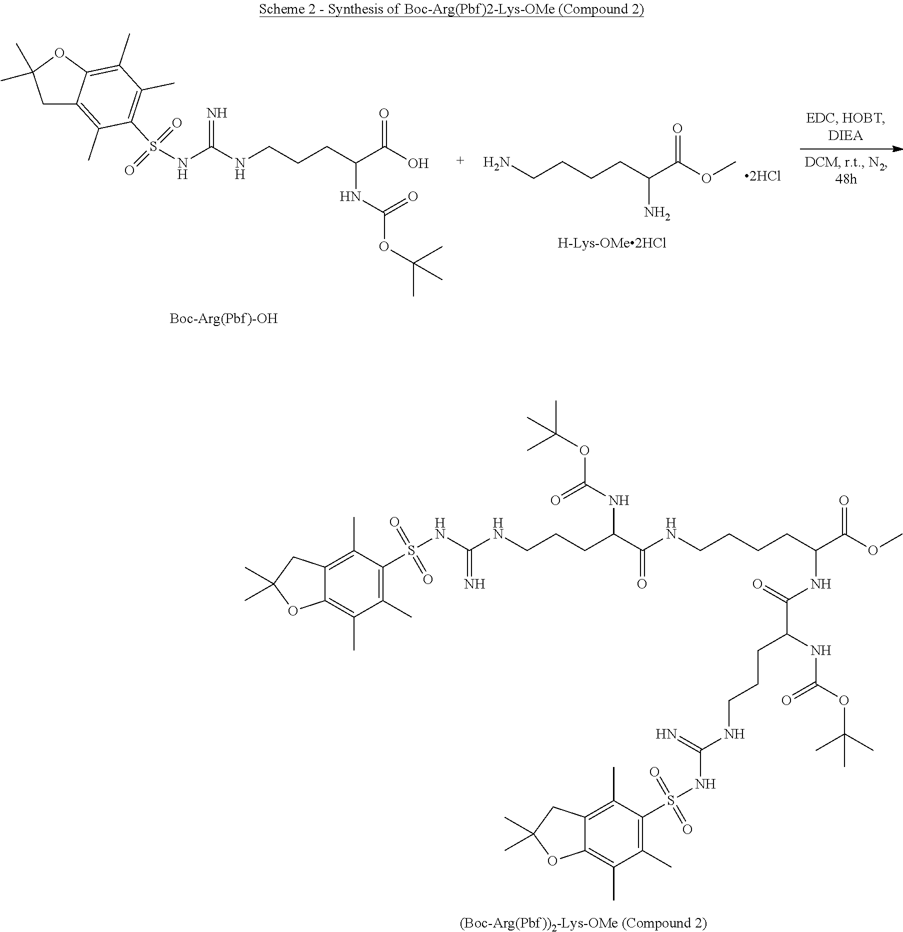

Synthesis of (Boc-Arg(Pbf))₂-Lys-OMe (Compound 2). H-Lys-OMe·2HCl (0.35 g, 1.5 mmol), Boc-Arg(Pbf)-OH (2 g, 3.8 mmol), EDC (1.16 g, 6.0 mmol) and HOBT (0.51 g, 3.8 mmol) were added to a round bottom flask. 30 mL DCM was added into above flask in nitrogen atmosphere and then DIEA (5.3 mL, 30 mmol) was slowly injected into the flask. The reaction was kept at room temperature for 48 hours with magnetic stirring. The solvent was removed by rotary evaporation. The resulting product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The product was further purified by column chromatography (MeOH/ethyl acetate/DCM=1:2:6) to give Compound 2. ¹H NMR (400 MHz, CDCl₃): δ 1.38 (s, 18H, Boc group); δ 1.46 (s, 12H, Pbf group); δ 1.53-1.92 (m, 14H, —CH₂CH₂CH₂C(NH—)CO—, —CH₂CH₂C(NH—)CO—); δ 2.09 (s, 6H, Pbf group); δ 2.50 (s, 6H, Pbf group); δ 2.57 (s, 6H, Pbf group); δ 2.95 (s, 4H, Pbf group); δ 3.09-3.37 (m, 6H, —CH₂NHC(NH)NH, —CH₂NHCO—); δ 3.70 (s, 3H, methyl easter group); δ 4.15-4.47 (m, 3H, —CONHCH(CH₂—)CO—).

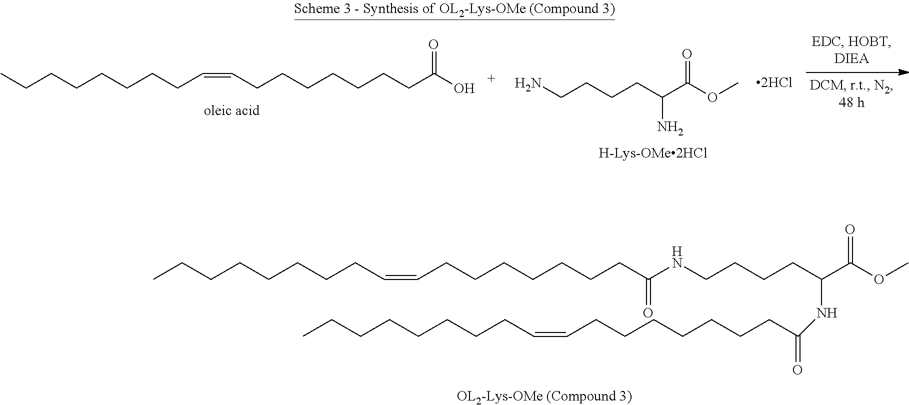 [SMILES: CCCCCCCC/C=C\CCCCCCCC(=O)O ; COC(=O)C(N)CCCCN ; CCCCCCCC/C=C\CCCCCCCC(=O)NCCCCC(NC(=O)CCCCCCC/C=C\CCCCCCCC)C(=O)OC]

Synthesis of OL₂-Lys-OMe (Compound 3). H-Lys-OMe·2HCl (0.35 g, 1.5 mmol), EDC (1.16 g, 6.0 mmol) and HOBT (0.51 g, 3.8 mmol) were added to a round bottom flask. Oleic acid (1.45 mL, 4.6 mmol) in 30 mL chloroform was added into the above flask in a nitrogen atmosphere and then DIEA (5.3 mL, 30 mmol) was added dropwise. The reaction was kept at room temperature for 48 hours with magnetic stirring. The solvent was removed by rotary evaporation. The resulting product was dissolved in 30 mL DCM and washed with saturated NaCl solution, 1 M HCl solution, and saturated NaHCO₃ solution, sequentially. The organic layer was collected and dried over anhydrous MgSO₄ overnight, filtered, and concentrated by rotary evaporation. The product was further purified by column chromatography (ethyl acetate/hexane from 1:5 to 1:1) on silica gel to give compound 3. ¹H NMR (400 MHz, CDCl₃): δ 0.88 (t, 6H, terminal methyl group); δ 1.22-1.37 (m, 42H, CH₃(CH₂)₆CH₂CH═CHCH₂(CH₂)₄CH₂—, —CONHCH₂CH₂CH₂CH₂—); δ 1.47-1.89 (m, 8H, —CONHCH₂CH₂CH₂CH₂—, —NHCOCH₂CH₂—); δ 2.01 (q, 8H, CH₃(CH₂)₆CH₂CH═CHCH₂(CH₂)₄CH₂—); δ 2.16 (t, 2H, —NHCOCH₂CH₂—); δ 2.23 (t, 2H, —NHCOCH₂CH₂—); δ 3.23 (t, 2H, —CONHCH₂CH₂CH₂CH₂—); δ 3.74 (s, 3H, methyl easter group); δ 4.59 (t, 1H, —CONHCH(CH₂—)CO—); δ 5.34 (dt, 4H, CH₃(CH₂)₆CH₂CH═CHCH₂(CH₂)₄CH₂—).

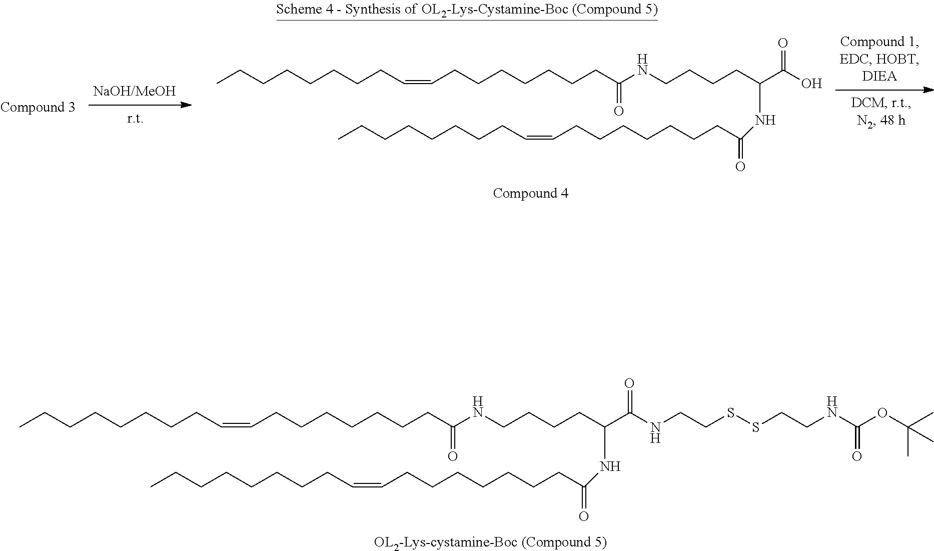 [SMILES: CCCCCCCC/C=C\CCCCCCCC(=O)NCCCCC(NC(=O)CCCCCCC/C=C\CCCCCCCC)C(=O)O ; CCCCCCCC/C=C\CCCCCCCC(=O)NCCCCC(NC(=O)CCCCCCC/C=C\CCCCCCCC)C(=O)NCCSSCCNC(=O)OC(C)(C)C]

Synthesis of Compound 4. Conjugation of the linker to the hydrophobic tail. To deprotect the carboxyl group, Compound 3 (1 g, 1.45 mmol) was dissolved in 20 mL 1 M NaOH MeOH solution at room temperature under stirring. The reaction was monitored by thin-layer chromatography (TLC). After the spot of compound 3 disappeared from the TLC plate, the solvent was evaporated and then the crude product was re-dissolved in 20 mL water. HCl solution (1M, 20 mL) was added dropwise into the water solution until pH=3. The product was extracted by DCM, dried by MgSO₄ overnight, and concentrated by rotary evaporation. Compound 4 was obtained without further purification.

Synthesis of OL₂-Lys-Cystamine-Boc (Compound 5). Compound 4 (0.8 g, 1.2 mmol) and Compound 1 (300 mg, 1.2 mmol) were dissolved in 20 mL DCM. The DCM solution was injected into a round bottom flask containing EDC (0.45 g, 2.4 mmol) and HOBT (0.32 g, 2.4 mmol) in nitrogen atmosphere. DIEA (1.9 mL, 12 mmol) was slowly injected into the flask. The reaction was proceeded at room temperature for 48 hours with magnetic stirring. The solvent was removed by rotary evaporation. The resulting product was dissolved in 30 mL DCM and washed with saturated NaCl solution, 1 M HCl solution, and saturated NaHCO₃ solution, sequentially. The organic layer was dried over anhydrous MgSO₄ overnight, filtered, and concentrated by rotary evaporation. The final product was further purified by column chromatography (ethyl acetate/chloroform from 1:5 to 1:1) on silica gel to give Compound 5. ¹H NMR (400 MHz, CDCl₃): δ 0.88 (t, 6H, terminal methyl group); δ 1.22-1.37 (m, 42H, CH₃(CH₂)₆CH₂CH═CHCH₂(CH₂)₄CH₂—, —CONHCH₂CH₂CH₂CH₂—); δ 1.45 (s, 9H, Boc group); δ 1.47-1.89 (m, 8H, —CONHCH₂CH₂CH₂CH₂—, —NHCOCH₂CH₂—); δ 2.01 (q, 8H, CH₃(CH₂)₆CH₂CH═CHCH₂(CH₂)₄CH₂—); δ 2.15 (t, 2H, —NHCOCH₂CH₂—); δ 2.23 (t, 2H, —NHCOCH₂CH₂—); δ 2.70-3.63 (m, 10H, —NHCH₂CH₂SSCH₂CH₂NH—, —CONHCH₂CH₂CH₂CH₂—); δ 4.42 (t, 1H, —CONHCH(CH₂—)CO—); δ 5.34 (dt, 4H, CH3(CH₂)₆CH₂CH═CHCH₂(CH₂)₄CH₂—).

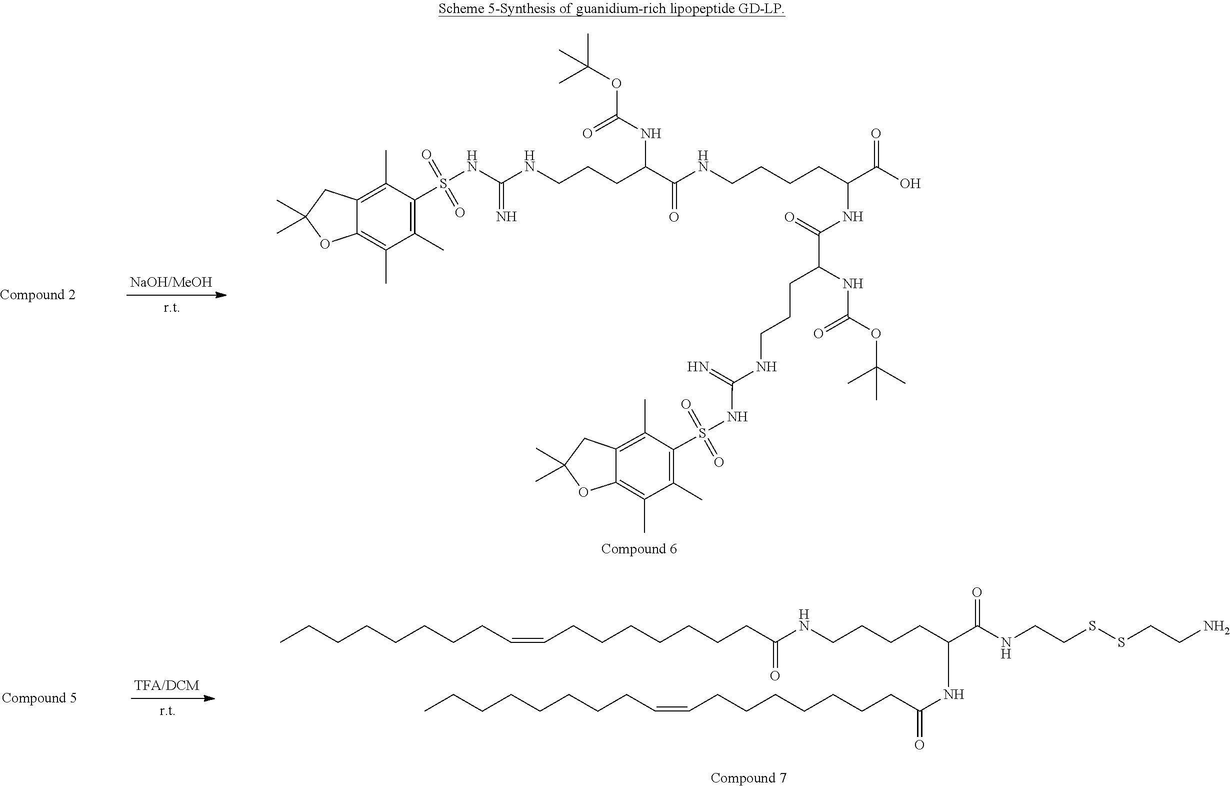

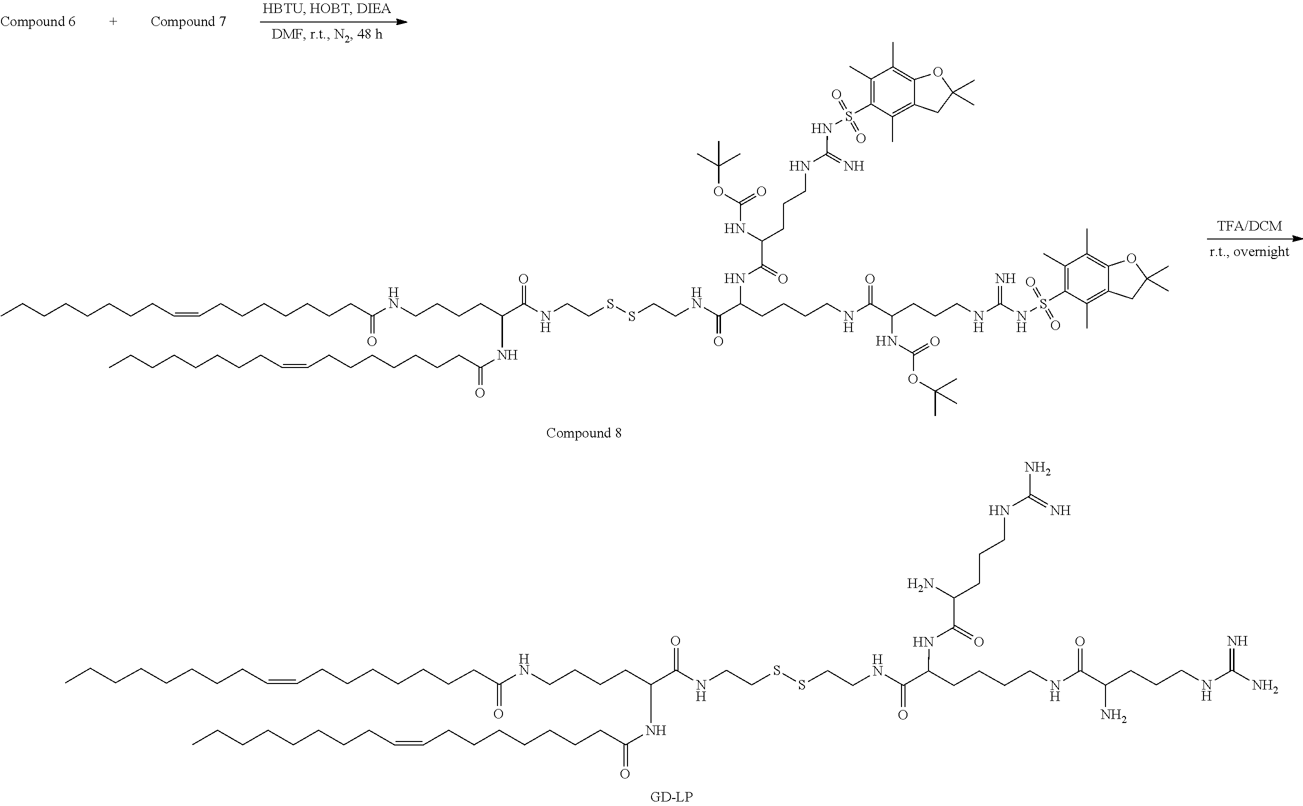

Synthesis of Compound 6. Compound 2 (1 g, 0.85 mmol) was deprotected with NaOH using the same procedures described in the synthesis of Compound 4. Compound 6 was used without further purification.

Synthesis of Compound 7. Compound 5 (0.77 g, 0.85 mmol) was dissolved in the mixture of 0.5 mL DCM and 0.5 mL TFA in a nitrogen atmosphere and the solution was stirred for 4 hours. The product was concentrated by rotary evaporation and precipitated in diethyl ether to give Compound 7. Compound 7 was used without further purification.

Synthesis of Compound 8. Compound 6 (470 mg, 0.59 mmol) and Compound 7 (686 mg, 0.59 mmol) were dissolved in 20 mL DMF and injected into a round bottom flask containing HBTU (334 mg, 0.88 mmol) and HOBT (119 mg, 0.88 mmol) in a nitrogen atmosphere. DIlEA (0.72 mL, 4.4 mmol) was slowly injected into the flask. The reaction was kept at room temperature for 24 hours with magnetic stirring. The solvent was removed by rotary evaporation. The product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The final product was further purified by column chromatography (MeOH/ethyl acetate/DCM=1:10:10) on silica gel to give compound 8. ¹H NMR (400 MHz, DMSO-d₆): δ 0.81 (t, 6H, terminal methyl group); δ 1.14-1.29 (m, 42H, CH3(CH₂)₆CH₂CHCHCH₂(CH₂)₄CH₂—, —CONHCH₂CH₂CH₂CH₂—); δ 1.33 (s, 18H, Boc group); δ 1.46 (s, 12H, Pbf group); δ 1.42-1.81 (m, 22H, —CH₂CH₂CH₂C(NH—)CO—, —CH₂CH₂C(NH—)CO—, —CONHCH₂CH₂CH₂CH₂—, —NHCOCH₂CH₂—); δ 1.94 (q, 8H, CH3(CH₂)₆CH₂CH═CHCH₂(CH₂)₄CH₂—); δ 2.02 (s, 6H, Pbf group); δ 2.08 (t, 2H, —NHCOCH2CH₂—); δ 2.15 (t, 2H, —NHCOCH2CH2-); δ 2.44 (s, 6H, Pbf group); δ 2.51 (s, 6H, Pbf group); δ 2.75 (t, 4H, —NHCH₂CH₂SSCH₂CH₂NH—); δ 2.88 (s, 4H, Pbf group); δ 3.05-3.62 (m, 12H, —CH₂NHC(NH)NH, —CH₂NHCO—, CONHCH₂CH₂CH₂CH₂—, —NHCH₂CH₂SSCH₂CH₂NH—); δ 4.03-4.45 (m, 4H, —CONHCH(CH₂—)CO—); δ 5.27 (dt, 4H, CH3(CH₂)₆CH₂CHCHCH₂(CH₂)₄CH₂—).

Synthesis of GD-LP. To expose amine and guanidium groups, Compound 8 (200 mg, 100 μmol) was dissolved in the mixture of 0.5 mL DCM and 0.5 mL TFA and the solution was stirred overnight in a nitrogen atmosphere. The product was concentrated by rotary evaporation and precipitated in diethyl ether to give GD-LP. ¹H NMR (400 MHz, CD₃OD): δ 0.92 (t, 6H, terminal methyl group); δ 1.14-1.46 (m, 42H, CH₃(CH₂)₆CH₂CH═CHCH₂(CH₂)₄CH₂—, —CONHCH₂CH₂CH₂CH₂—); δ 1.46-1.86 (m, 22H, —CH₂CH₂CH₂C(NH—)CO—, —CH₂CH₂C(NH—)CO—, —CONHCH₂CH₂CH₂CH₂—, —NHCOCH₂CH₂—); δ 2.04 (q, 8H, CH3(CH₂)₆CH₂CH═CHCH₂(CH₂)₄CH₂—); δ 2.19 (t, 2H, —NHCOCH2CH₂—); δ 2.28 (t, 2H, —NHCOCH2CH2-); δ 2.86 (t, 4H, —NHCH₂CH₂SSCH₂CH₂NH—); δ 3.12-3.79 (m, 12H, —CH₂NHC(NH)NH, —CH₂NHCO—, CONHCH₂CH₂CH₂CH₂—, —NHCH₂CH₂SSCH₂CHNH—); δ 3.85-4.41 (m, 4H, —CONHCH(CH₂—)CO—); δ 5.36 (dt, 4H, CH3(CH₂)₆CH₂CHCHCH₂(CH₂)₄CH₂—).

### Example 2—Preparation of Lipoplex Incorporating Positively Charged Lipopeptides

Preparation of Lipoplex. The empty liposomal particles (ELP) were prepared by self-assembly of GD-LP. 4.75 mg GD-LP and 0.25 mg DSPE-PEG2000 were dissolved in 25 μL DMF. The weight percent ratio of GD-LP/DSPE-PEG2000 was 95/5. Then the solution was added to 1 mL sodium acetate buffer (pH=5.2) under sonication. Another 30 s of sonication was applied to yield ELP. RNP solution (1 mg/mL, molar ratio of Cas9:sgRNA=1:1) was prepared by mixing Cas9 nuclease and sgRNA in PBS and incubated at room temperature for 5 min. The ELP solution was mixed with RNP solution in PBS to form RNP loaded lipoplexes (RNP-lipoplexes). RNP-lipoplex was incubated for another 5 min before use. DNA and mRNA loaded lipoplexes were prepared in the same way by using DNA and mRNA instead of RNP, respectively. Furthermore, pre-mixed RNP and ssODN were mixed simultaneously with ELP, yielding RNP/ssODN loaded lipopeptide-based lipoplex (RNP/ssODN-lipoplex) to enable precise gene editing via the homology-directed repair (HDR) process.

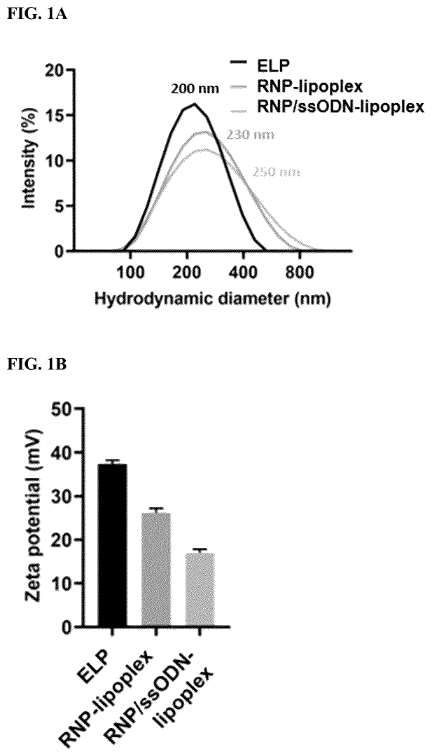

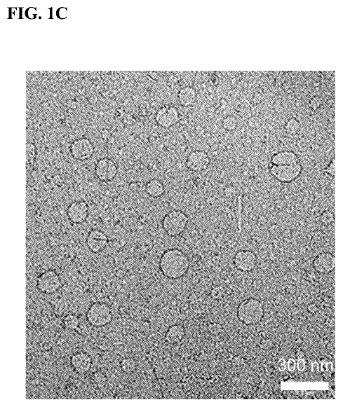

Characterization of LNP. The hydrodynamic diameters and zeta potentials of various lipoplexes were measured by ZetaSizer Nano ZS90 (Malvern Panalytical Ltd) and analyzed using Zetasizer software v7.01. The morphology of lipoplexes were characterized by transmission electron microscopy (FEI Tecnai 12, 120 keV) and analyzed by ImageJ. The size of ELP was found to be 198.5 nm (FIG. 1A) with a high positive surface charge of +37.4 mV (FIG. 1B) due to the protonated amine and guanidinium groups. The zeta potential of the RNP (Cas9 protein and sgRNA were first pre-mixed at a molar ratio of 1 to 1) was 18.3 mV. Following complexation of the ELP with Cas9 RNP, the mean particle size increased from 198.5 to 231.3 nm (FIG. 1A) and zeta potential dropped from +37.4 to +26.2 mV compared with ELP (FIG. 1B), indicating the successful loading of RNP on ELP. The morphology studied by transmission electron microscopy (TEM) showed that RNP-lipoplex has a spherical shape (FIG. 1C). RNP/ssODN-lipoplex had a relatively bigger size (249.3 nm) and lower zeta potential (17.1 mV, FIGS. 1A-1B) attributed to more anionic payload absorbed on the lipoplex surface.

### Example 3—Biological Activity Results for Guanidinium Lipoplexes

### Lipoplex-Mediated In Vitro Genome Editing and Transfection

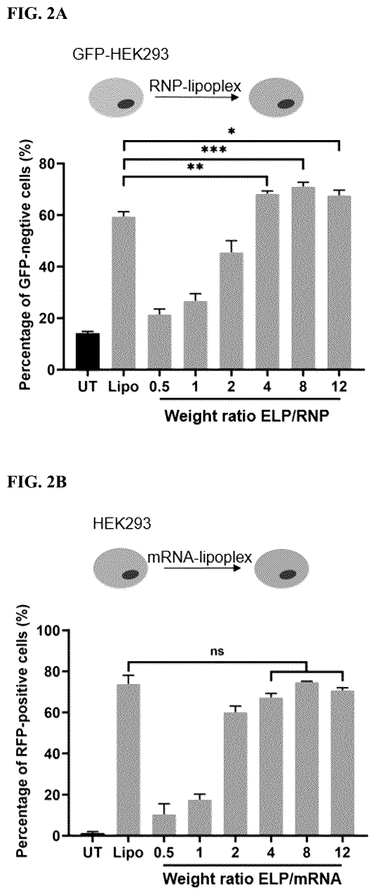

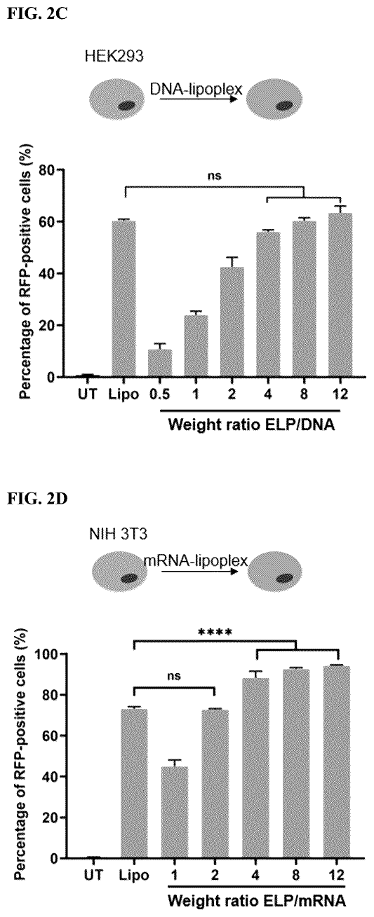

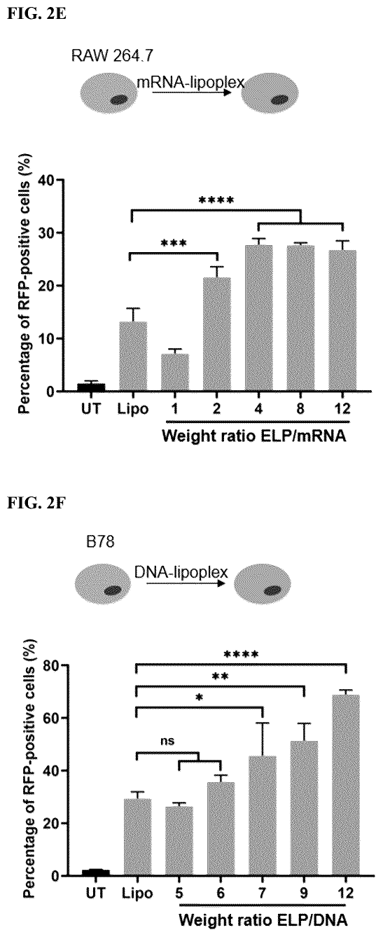

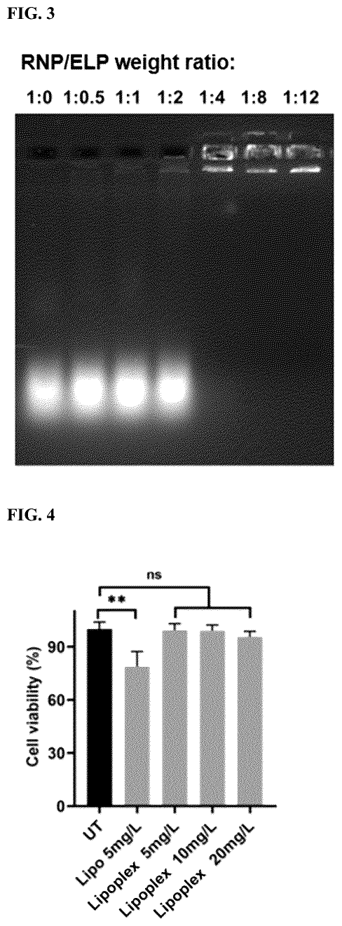

The in vitro genome editing effects of the lipoplexes of Example 2 were studied using human embryonic kidney (HEK 293) cells. Green fluorescent protein (GFP)-expressing HEK 293 cells were treated with ELP complexed with Cas9 RNP targeting the GFP gene cassette. Successful delivery of RNP will result in GFP gene knockdown and subsequently suppress GFP expression which can be quantified by flow cytometry. Firstly, the optimized weight ratio of RNP-lipoplex was screened by complexing ELP with RNP in different weight ratios. The gene editing efficiencies of RNP-LNP formulations with ELP/RNP weight ratios ranging from 4 to 12 were significantly higher than Lipofectamine 2000 (FIG. 2A). The binding ability of ELP with RNP was also verified by gel electrophoresis (FIG. 3). The RNP mobility in the RNP-lipoplex nanocomplexes was completely retarded at an ELP/RNP weight ratio of 4, indicating efficient RNP complexation with the lipopeptide. Considering the high loading content and efficient gene editing observed, RNP-lipoplex with a weight ratio of 4:1 was selected for following experiments. GD-LP provides plenty of guanidinium residues on the surface of ELP, leading to a high RNP loading content of up to 20% via multivalency interactions between guanidinium groups and the anionic sidechains of proteins and phosphate backbone of sgRNA from the RNP payloads. Significantly improved gene editing efficiency in the RNP delivery may also be attributed to the bidentate hydrogen bond formation between guanidium groups and cell-surface anions from phospholipids, fatty acids, proteins, and HSPGs.¹³ We tested the transfection efficiency of the lipoplexes prepared in Example 2 by delivering red fluorescence protein (RFP)-mRNA and RFP-plasmid DNA in HEK 293 cells. Lipoplex achieved a similar transfection efficiency with Lipofectamine 2000 (FIGS. 2B-2C). However, lipoplex exhibited significantly higher transfection efficiency than Lipofectamine 2000 for the delivery of RFP-mRNA in NIH 3T3 cells (FIG. 2D) and RAW 264.7 cells (FIG. 2E) and for the delivery of RFP-plasmid DNA in B78 melanoma cells (FIG. 2F).

Cytotoxicity of lipoplex was evaluated via an MTT (3-(4,5-dimethylthiazol-2-yl)-2,5-diphenyltetrazolium bromide) cell-proliferation assay in HEK 293 cells. Cells were treated with different concentrations of lipoplex (5-20 μg/mL) or lipofectamine 2000 (5 μg/mL). No obvious cytotoxicity of lipoplex was detected in a wide range of concentrations of lipoplex, which reveals the excellent biocompatibility of this lipoplex (FIG. 4). However, compared with the untreated group, a decline in cell viability was detected in the cells treated with Lipofectamine 2000, indicating severe cytotoxicity.

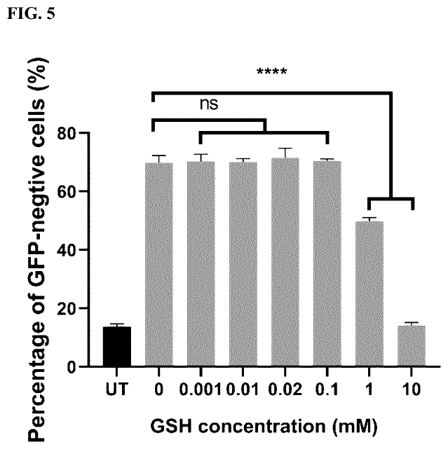

A disulfide bond was integrated into the lipopeptide to facilitate payload release in the cytosol with a high GSH concentration (1-10 mM).¹ Meanwhile, the RNP-lipoplex nanocomplexes were expected to remain stable in the extracellular spaces (GSH concentration, 0.001-0.02 mM)¹⁸ before cell internalization. To study the GSH-responsive behavior, RNP-lipoplex was incubated with GFP-HEK cells in culture medium with a GSH concentration ranging from 0 to 10 mM. As shown in FIG. 5, the editing efficiency was not affected at GSH concentrations of 0.1 mM or lower, indicating that RNP-lipoplex was stable at a GSH concentration up to 0.1 mM. A significant decrease in the editing efficiency was observed at a GSH concentration of 1 mM or higher, suggesting that the RNP-lipoplex nanocomplexes were disrupted before cell internalization. This study showed that the RNP-lipoplex nanocomplexes were stable at extracellular spaces, but could break down and release payloads effectively in the GSH-rich cytosol.

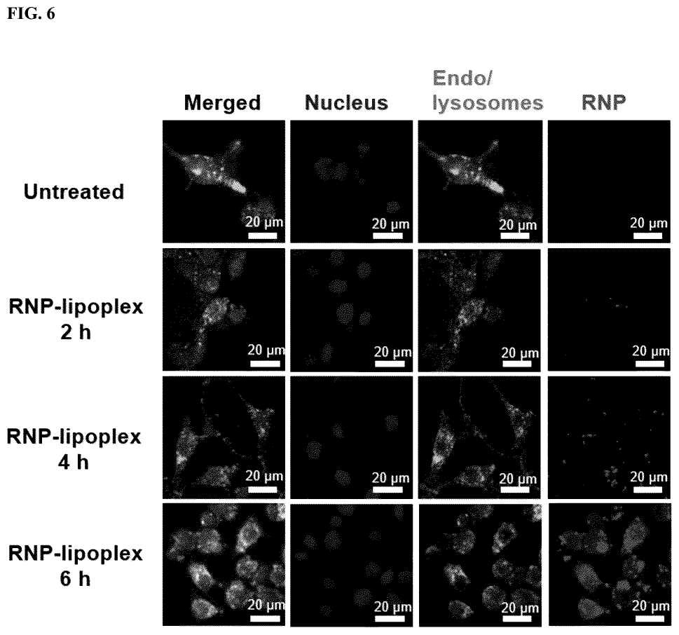

The intracellular trafficking of Cas9 RNP delivered by lipoplex was studied by confocal laser scanning microscopy (CLSM, FIG. 6). After the cells were incubated with RNP-lipoplex, the Atto550-labeled RNP was mainly co-localized with endo/lysosomes 2 and 4 hours post treatment, indicating the internalization of RNP-lipoplex via endocytosis. After 6 hours of incubation, more RNPs were found in the cytosol, not overlapping with endo/lysosomes, indicating successful endosomal escape induced by the lipopeptide. Overlapping of Cas9 RNPs with cell nuclei was also observed at 6 hours post-treatment, demonstrating the nuclear transportation of RNP via the nuclear localization signal (NLS) tags on the RNP.

### In Vivo Genome Editing on Ai14 Mice Via Intramuscular Injection of RNP-LNP

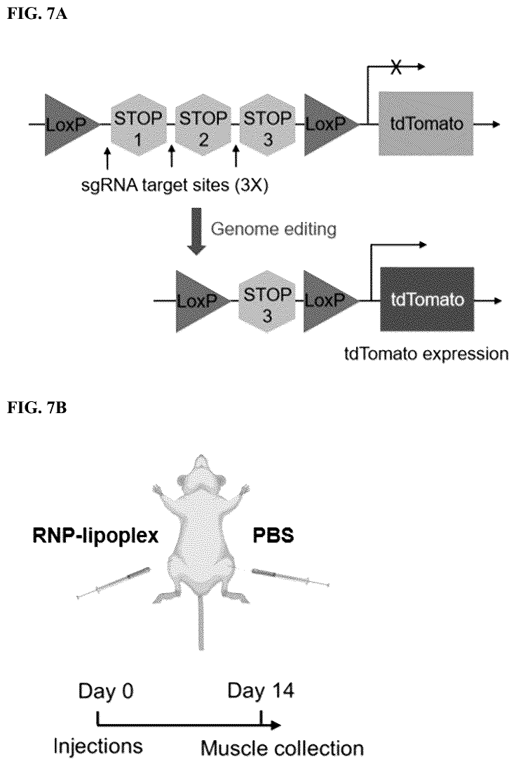

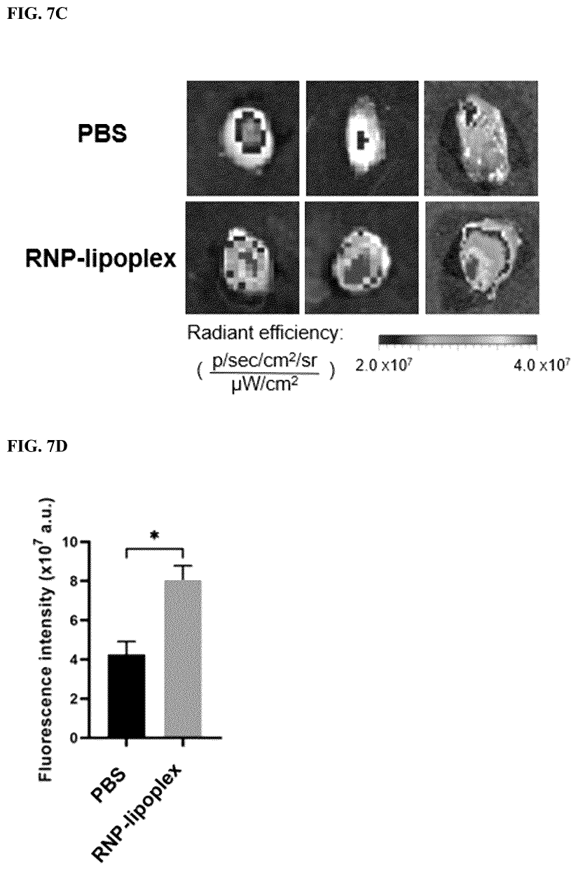

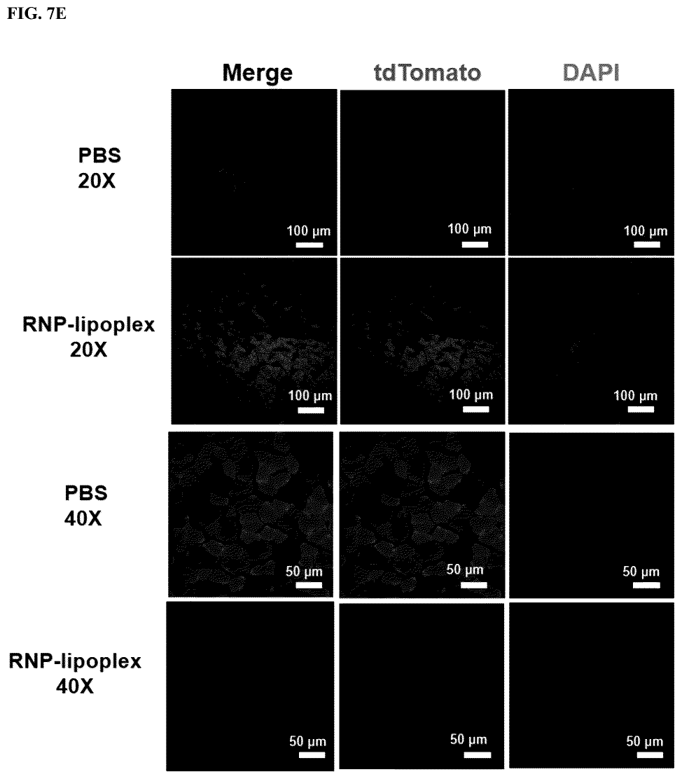

After confirming the gene delivery capability of LNP in vitro, we next tested the gene editing efficiency of RNP-lipoplex in vivo. Transgenic Ai14 mice have a LoxP-flanked STOP cassette preventing transcription of a reporter protein (i.e., tdTomato) driven by a CAG promoter.^{4, 19} tdTomato can be induced via RNP-mediated excision of the STOP cassette (FIG. 7A). Thus, the editing efficiency in Ai14 mice can easily be observed and measured by tdTomato fluorescence. RNP-lipoplex and PBS were injected into the left and right tibialis anterior muscles of each mouse, respectively. Mice were sacrificed after 14 days. Their tibialis anterior muscles were isolated (FIG. 7B) and imaged by an In Vivo Imaging System (IVIS) to visualize and quantify the tdTomato expression. The tdTomato fluorescence intensity of the muscle treated with RNP-lipoplex was two folds of the PBS treated counterpart (FIG. 7C-7D). Furthermore, the immunofluorescence staining of the tibialis anterior muscle sections demonstrated strong tdTomato expression after treatment with RNP-lipoplex (FIG. 7E). The pattern of more tdTomato positive cells in the center of the muscle than in surrounding regions might be attributed to the diffusion of RNP-lipoplex from injection site to adjacent myofibers. Overall, the abundant tdTomato expression induced by RNP-lipoplex in Ai14 mice demonstrated efficient in vivo genome editing in the muscles via lipoplex-mediate RNP delivery.

### Therapeutic Effects of RNP/ssODN-Lipoplex in Mdx Mice by Intramuscular Injection

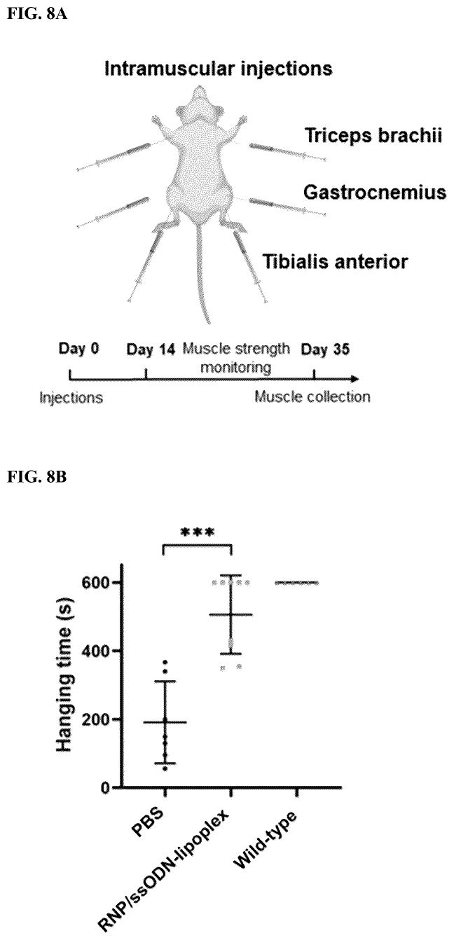

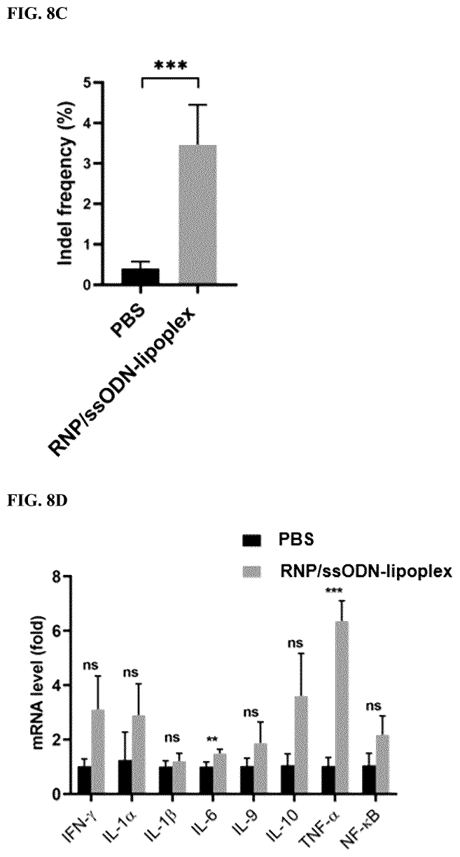

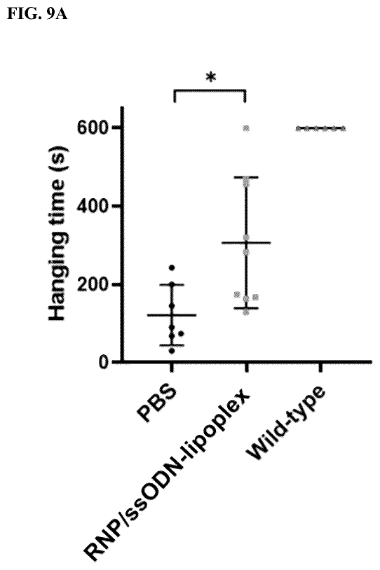

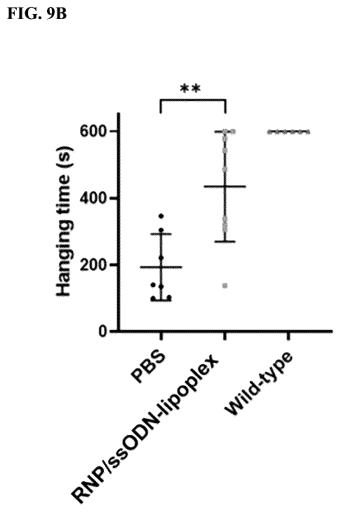

Encouraged by the successful genome editing in Ai14 mice, we next explored the therapeutic effect for treating genetic muscular disease by RNP/ssODN-lipoplex-mediated gene editing. DMD is a genetic disease characterized by progressive muscle degeneration resulting from mutations in the DMD gene coding for the dystrophin protein.²⁰ The mdx mice were designed to carry a nonsense point mutation (C to T transition) in exon 23 that aborted full-length dystrophin expression.²¹ Delivery of RNP together with a repair template targeting this region could disrupt the point mutation or correct the gene via NHEJ or HDR^{2, 22} processes. Therefore, the dystrophin expression can be recovered to restore the muscle strength. Four-week-old male mdx mice were intramuscularly injected with RNP/ssODN-lipoplex in the tibialis anterior, gastrocnemius, and triceps brachii muscles on both the left and right sides (FIG. 8A). mdx mice injected with PBS and wild-type mice without injections were used as a negative control and positive control, respectfully. Four-limb hanging tests were performed weekly to monitor the muscle strength in mdx mice and wild-type mice. RNP/ssODN-lipoplex treated mdx mice showed a continuously improved hanging time performance over 2 to 4 weeks post-injection, indicating accumulative therapeutic benefit from gene editing (FIG. 8B, FIG. 9). Remarkably, 4 weeks post-injection, over half of the mice (5 out of 9) treated with RNP/ssODN-lipoplex showed comparable hanging time (over 10 min) with wild-type mice, indicating great relief from muscle weakness. The rest of the lipoplex-treated mice also benefited from RNP/ssODN-lipoplex treatment with better hanging time performance than the PBS-treated group (FIG. 8B). Sanger sequencing revealed 3.5% of indels in the targeted region of genomic DNA from the lipoplex-injected muscles (FIG. 8C). To test if there is severe immune response from intramuscular administration of RNP/ssODN-lipoplex, a panel of inflammatory cytokines were studied by quantitative reverse transcription PCR (RT-qPCR) comparing with the PBS-treated group (negative control, FIG. 8D). For most of the tested cytokines (except IL-6 and TNF-α), the average mRNA levels increased after RNP/ssODN-lipoplex treatment, but there was no statistical difference compared with the PBS control. The level of IL-6 increased 50% showing a significant difference compared with the PBS control group, while the level of TNF-α exhibited a 5.3-fold increase. The increase in TNF-α and IL-6 in the RNP/ssODN-lipoplex treated group may be attributed to the clearance of the RNP/ssODN-LNP by immune cells (e.g., macrophage) in muscles.^{2, 23}

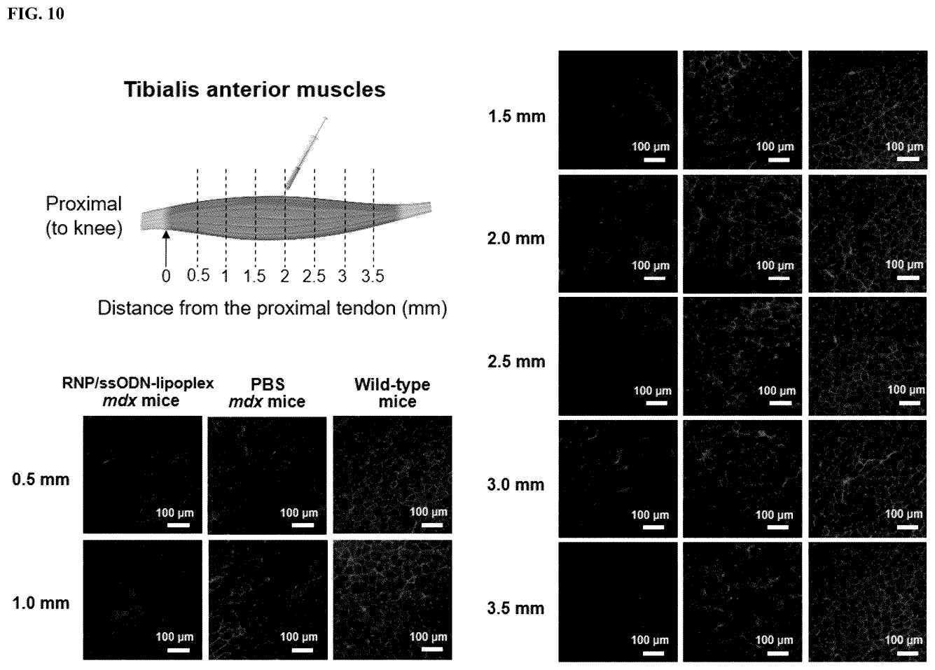

To demonstrate the outcome of therapeutic genome editing at a protein level, restored dystrophin expression was verified by immunostaining. Immunostaining of dystrophin was performed on tissue sections collected at different distances from the tendon in the tibialis anterior from RNP/ssODN-lipoplex treated mdx mice (FIG. 10). The images by CLSM showed that the restored dystrophin expression was most abundant in the center part of muscle tissue surrounding the injection site (middle of the skeletal muscle, FIG. 10). Also, expression of dystrophin can be observed about 2 mm away from the injection site which was attribute to the diffusion of lipoplex. In addition, the Hematoxylin and eosin stain (H&E) staining and Masson's staining were performed to verify the reduction of fibrosis in three types of muscles (i.e., tibialis anterior, gastrocnemius and triceps brachii) treated by RNP/ssODN-lipoplex. The fibrosis level was reduced significantly in all three types of muscles treated by RNP/ssODN-lipoplex in comparison with the PBS group (FIGS. 11A-11B).

### Example 4—Preparation of Additional Lipopeptides with Disulfide Linkages

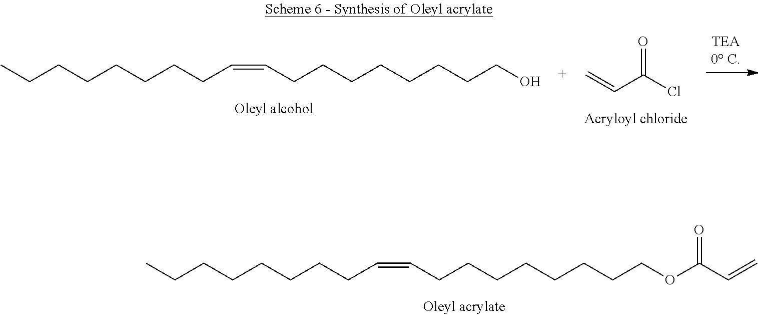

Oleyl alcohol (1.90 g, 7.08 mmol) and TEA (1.23 g, 12.16 mmol) were added to a round bottom flask and dissolved in 20 mL DCM. The mixture was cooled down to 0° C. in an ice bath. Acryloyl chloride (1.00 g, 11.05 mmol) was dissolved in 10 mL DCM and added dropwise to the above solution and the reaction proceeded at 0° C. for 3 hours under stirring. The mixture was washed with 20 mL DI water three times. The organic layer was collected, dried by anhydrous MgSO₄ overnight, filtered, and concentrated by rotary evaporation. The product was further purified by column chromatography (Hexane/DCM=1/1, v/v) on silica gel. ¹H NMR (400 MHz, CD₃OD): δ 0.87 (t, 3H, terminal methyl group); δ 1.21-1.43 (m, 22H, CH₃(CH₂)₆CH₂CH═CHCH₂(CH₂)₅CH₂CH₂OCOCH═CH₂); δ 1.66 (m, 2H, —CH₂CH₂OCOCH═CH₂); δ 2.02 (q, 4H, —CH₂CH═CHCH₂—); δ 4.15 (t, 2H, —CH₂OCOCH═CH₂); δ 5.35 (m, 2H, —CH₂CH═CHCH₂—); δ 5.83-6.38 (m, 3H, acrylate group).

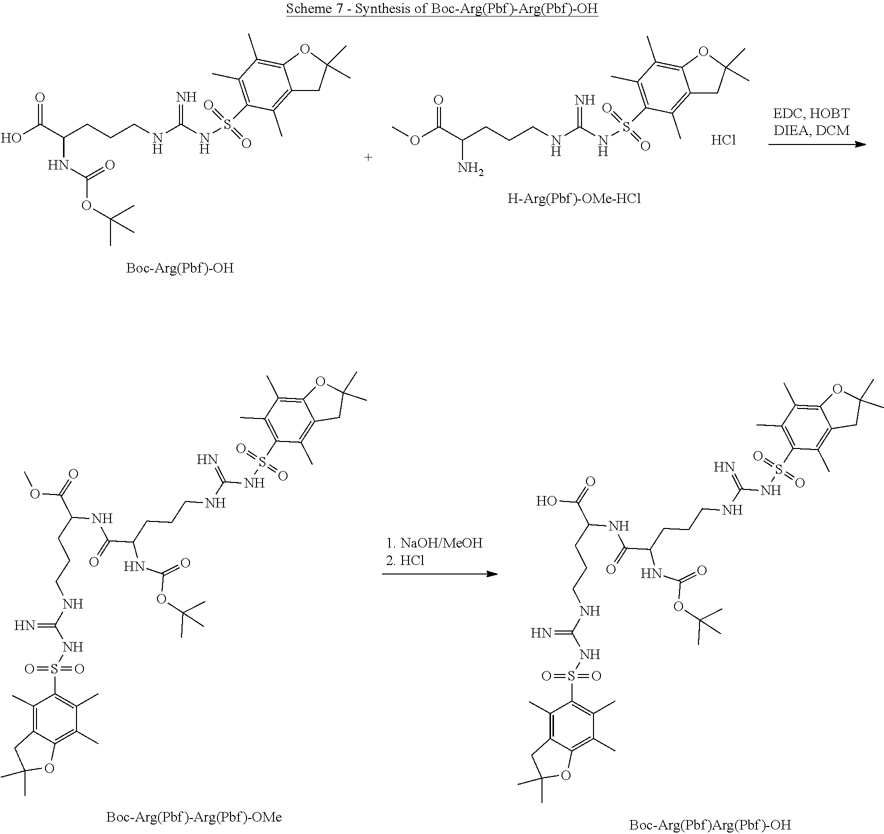

Synthesis of Boc-Arg(Pbf)-Arg(Pbf)-OH. H-Arg(Pbf)-OMe-HCl (0.91 g, 1.9 mmol), Boc-Arg(Pbf)-OH (1 g, 1.9 mmol), EDC (0.58 g, 3.0 mmol), and HOBT (0.26 g, 1.9 mmol) were added to a round bottom flask. DCM (30 mL) was added into the flask in a nitrogen atmosphere and then DIlEA (2.7 mL, 15 mmol) was slowly injected into the flask. The reaction was kept at 20° C. for 48 hours with magnetic stirring. The solvent was removed by rotary evaporation. The resulting product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The product was further purified by column chromatography (MeOH/DCM=1/10, v/v) to give Boc-Arg(Pbf)-Arg(Pbf)-OMe. ¹H NMR (400 MHz, CDCl₃): δ 1.38 (s, 9H, Boc group); δ 1.45 (s, 12H, Pbf group); δ 1.53-1.92 (m, 8H, —CH₂CH₂CH₂NH(NH)NH—); δ 2.09 (s, 6H, Pbf group); δ 2.50 (s, 6H, Pbf group); δ 2.57 (s, 6H, Pbf group); δ 2.95 (s, 4H, Pbf group); δ 3.08-3.29 (m, 4H, —CH₂CH₂CH₂NH(NH)NH—); δ 3.70 (s, 3H, methyl easter group); δ 4.25-4.46 (m, 2H, —CONHCH—).

To deprotect the carboxyl group, Boc-Arg(Pbf)-Arg(Pbf)-OMe (1 g, 1.05 mmol) was dissolved in 20 mL 1 M NaOH MeOH solution at 20° C. under stirring. The reaction was monitored by thin-layer chromatography (TLC). After the spot of (Boc-Arg(Pbf))2-Lys-OMe disappeared from the TLC plate, the solvent was evaporated and then the crude product was re-dissolved in 20 mL water. HCl solution (1M, 20 mL) was added dropwise into the water solution until pH=3. The product was extracted by DCM, dried by MgSO₄ overnight, and concentrated by rotary evaporation. Boc-Arg(Pbf)-Arg(Pbf)-OH was used without further purification.

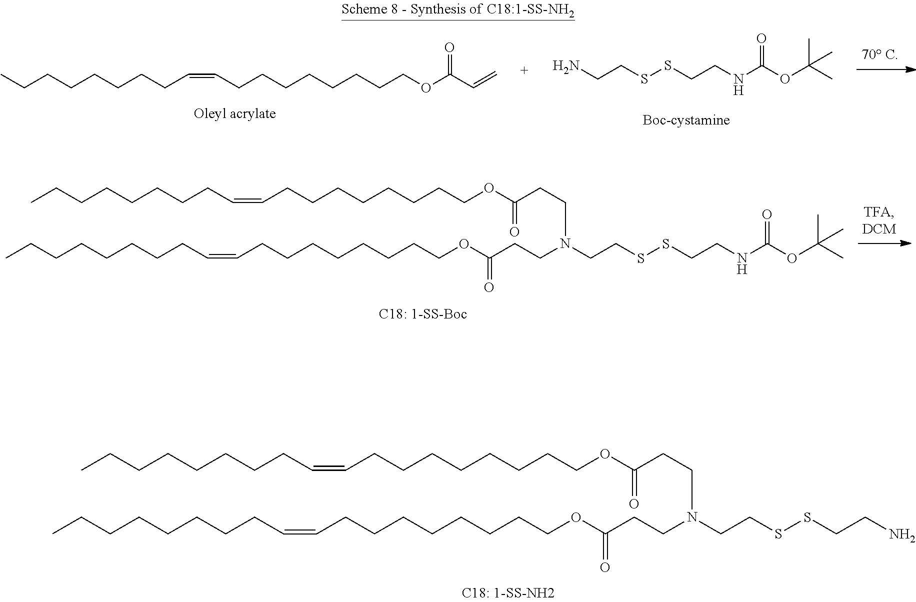

Synthesis of C18:1-SS-NH₂. Oleyl acrylate (2.00 g, 6.20 mmol) and Boc-cystamine (0.65 g, 2.58 mmol) were added to a round bottom flask. The reaction mixture was stirred at 70° C. for 3 days. The product was further purified by column chromatography (MeOH/DCM=1/10, v/v) to give C18:1-SS-Boc. ¹H NMR (400 MHz, CDCl3): δ 0.88 (t, 6H, terminal methyl group); δ 1.18-1.37 (m, 44H, CH₃(CH₂)₆CH₂CH═CHCH₂(CH₂)₅CH₂—); δ 1.45 (s, 9H, Boc group); δ 1.54-1.68 (m, 12H, —CH₂CH₂OCO—, —CH₂CH═CHCH₂—); δ 2.33-2.49 (m, 10H, —CH₂O(CO)CH₂—, —CH₂N(CH₂—)CH₂—); δ 2.70-2.83 (m, 6H, —CH₂N(CH₂—)CH₂CH₂SSCH₂CH₂NH—); δ 3.11 (t, 4H, —CH₂OCO—); δ 4.05 (dt, 4H, —CH₂CH═CHCH₂—).

C18:1-SS-Boc (1.00 g, 1.11 mmol) was dissolved in the mixture of 0.5 mL DCM and 0.5 mL TFA in a nitrogen atmosphere, and the solution was stirred for 4 hours. The product was concentrated by rotary evaporation and precipitated in diethyl ether to give C18:1-SS-NH₂.

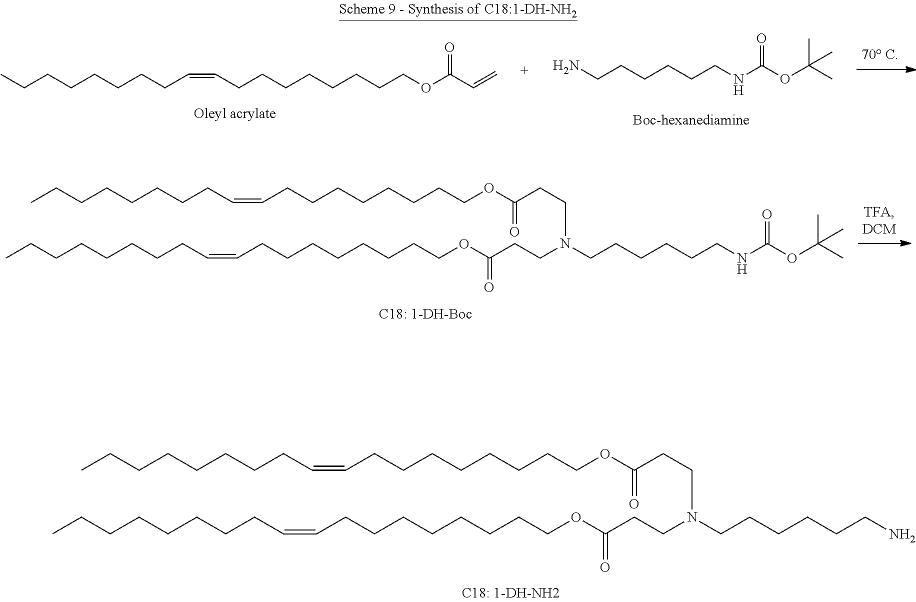

Synthesis of C18:1-DH-NH₂. For the lipopeptide without the disulfide bond, Oleyl acrylate (2.00 g, 6.20 mmol) and Boc-hexanediamine (0.56 g, 2.58 mmol) were added to a round bottom flask. The reaction mixture was stirred at 70° C. for 3 days. The product was further purified by column chromatography (MeOH/DCM=1/10, v/v) to give C18:1-DH-Boc. ¹H NMR (400 MHz, CDCl₃): δ 0.88 (t, 6H, terminal methyl group); δ 1.18-1.37 (m, 50H, CH3(CH₂)₆CH₂CH═CHCH₂(CH₂)₅CH₂—, —CH₂N(CH₂—)CH₂CH₂CH₂CH₂CH₂CH₂NH—); δ 1.45 (s, 9H, Boc group); δ 1.54-1.68 (m, 14H, —CH₂CH₂OCO—, —CH₂CH═CHCH₂—, —CH₂N(CH₂—)CH₂CH₂CH₂CH₂CH₂CH₂NH—); δ 2.33-2.49 (m, 10H, —CH₂O(CO)CH₂—, —CH₂N(CH₂—)CH₂); δ 2.90 (m, 2H, —CH₂N(CH₂—)CH₂CH₂CH₂CH₂CH₂CH₂NH—); δ 3.11 (t, 4H, —CH₂OCO—); δ 4.05 (dt, 4H, —CH₂CH═CHCH₂—).

C18:1-DH-NH₂ was obtained by deprotection of the Boc group with the same procedures described in the synthesis of C18:1-SS-NH₂.

Other linker-tail conjugates (shown in Scheme 10 below) were synthesized by Michael addition between corresponding acrylate tails and linkers with the same procedures described for the synthesis of C18:1-SS-NH₂ or C18:1-DH-NH₂.

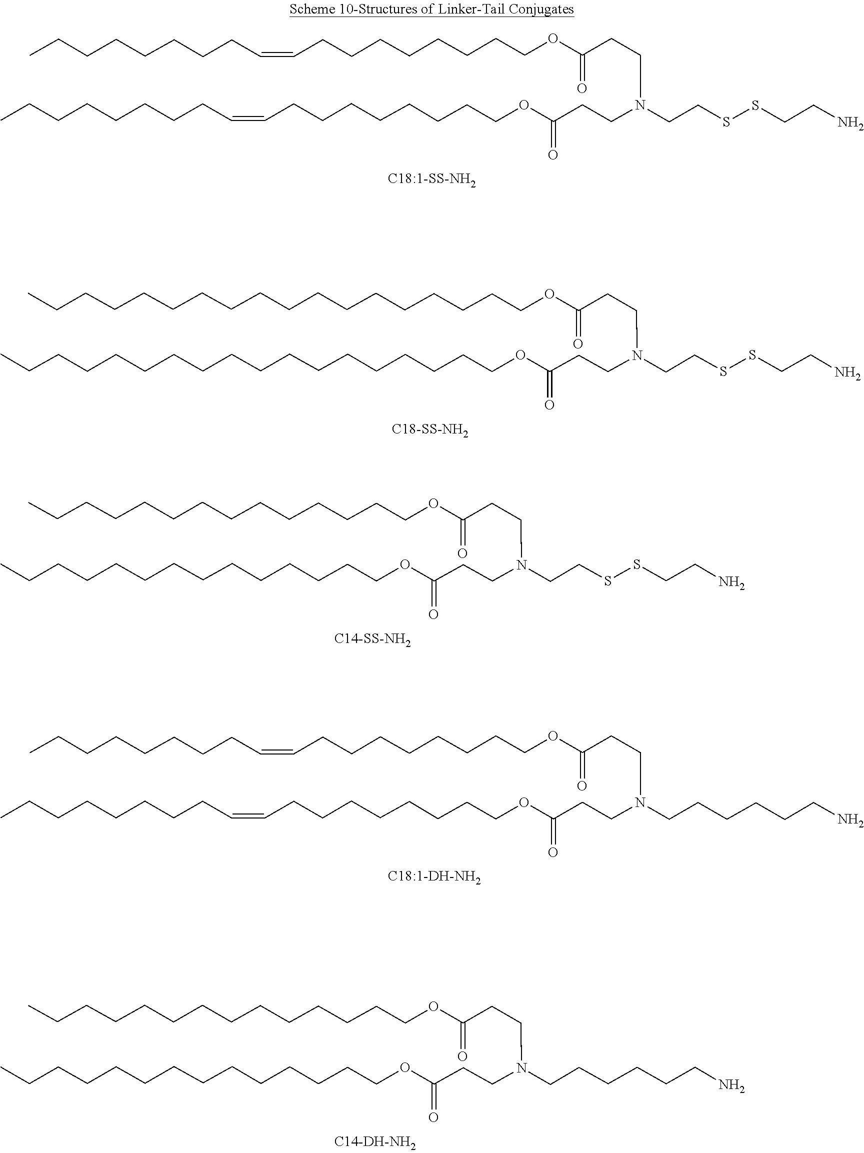 [SMILES: CCCCCCCC/C=C\CCCCCCCCOC(=O)CCN(CCSSCCN)CCC(=O)OCCCCCCCC/C=C\CCCCCCCC ; CCCCCCCCCCCCCCCCCCOC(=O)CCN(CCSSCCN)CCC(=O)OCCCCCCCCCCCCCCCCCC ; CCCCCCCCCCCCCCOC(=O)CCN(CCSSCCN)CCC(=O)OCCCCCCCCCCCCCC ; CCCCCCCC/C=C\CCCCCCCCOC(=O)CCN(CCCCCCN)CCC(=O)OCCCCCCCC/C=C\CCCCCCCC ; CCCCCCCCCCCCCCOC(=O)CCN(CCCCCCN)CCC(=O)OCCCCCCCCCCCCCC]

Additional lipopeptides (Scheme 11) were synthesized as described below.

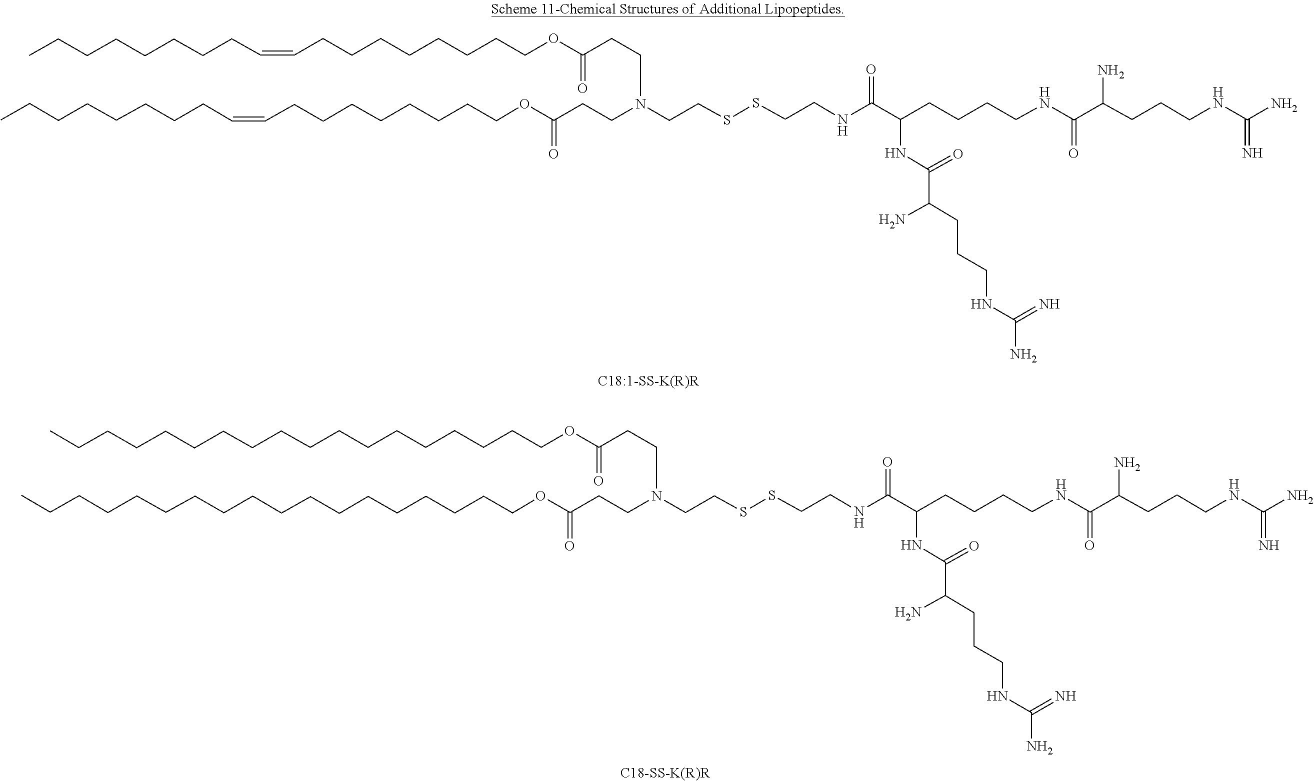 [SMILES: CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC ; CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC ; "C18-SS-K(R)R"]

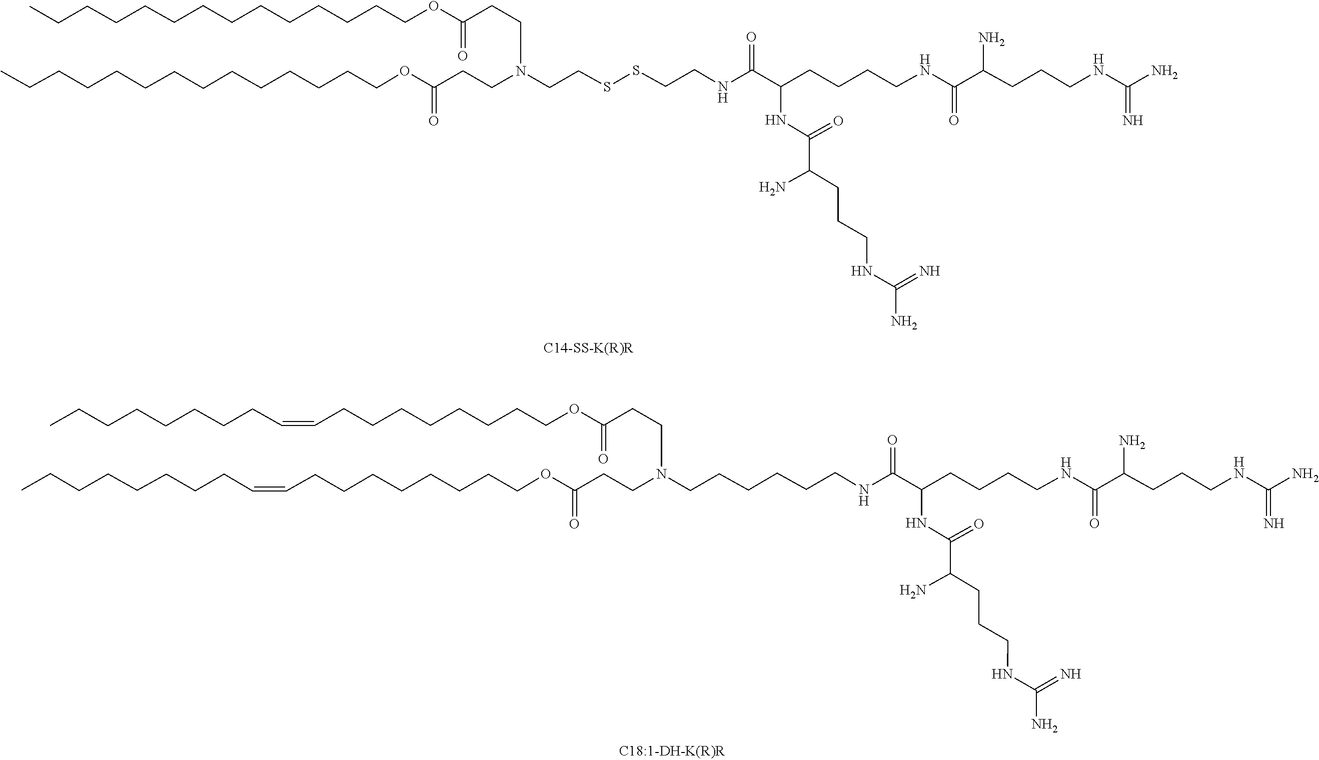 [SMILES: CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC ; CCCCCCCC/C=C\CCCCCCCCOC(=O)CCN(CCCCCCNC(=O)C(CCCCNC(=O)C(N)CCCNC(=N)N)NC(=O)C(N)CCCNC(=N)N)CCC(=O)OCCCCCCCC/C=C\CCCCCCCC ; "C14-SS-K(R)R"]

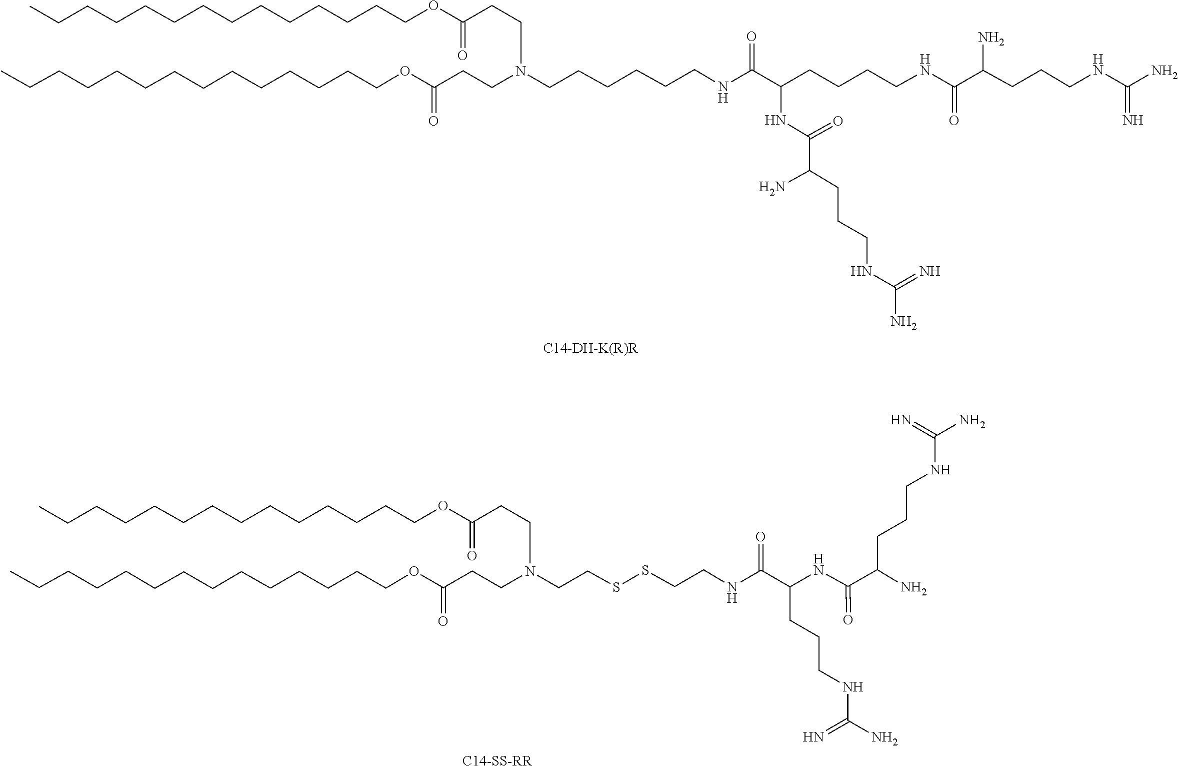 [SMILES: CCCCCCCCCCCCCCOC(=O)CCN(CCCCCCNC(=O)C(CCCCNC(=O)C(N)CCCNC(=N)N)NC(=O)C(N)CCCNC(=N)N)CCC(=O)OCCCCCCCCCCCCCC ; "C14-DH-K(R)R" ; CCCCCCCCCCCCCCOC(=O)CCN(CCSSCCNC(=O)C(CCCNC(=N)N)NC(=O)C(N)CCCNC(=N)N)CCC(=O)OCCCCCCCCCCCCCC ; "C14-SS-RR"]

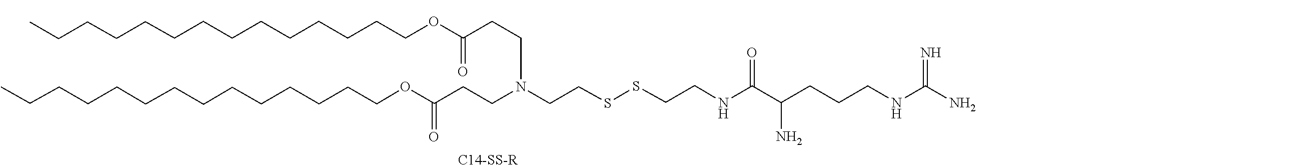

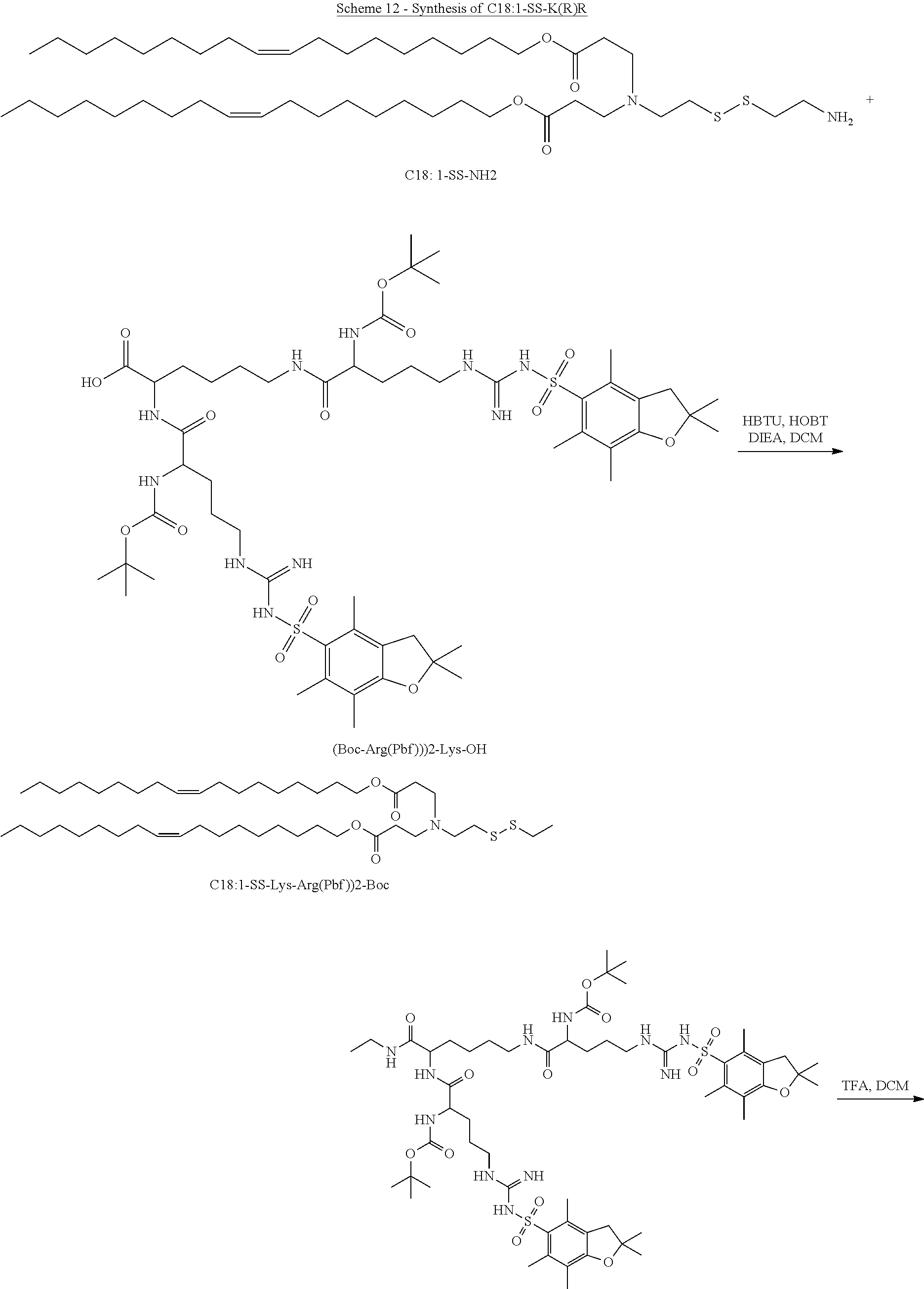

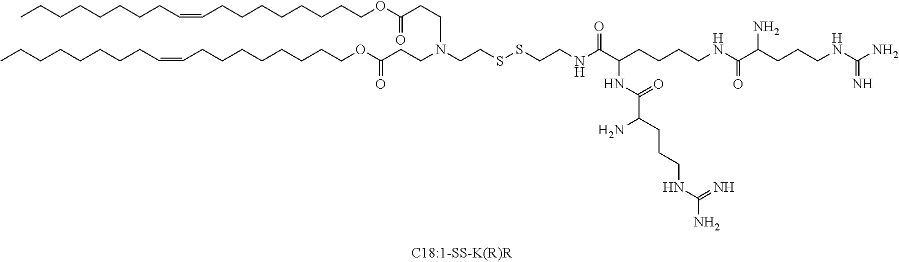 [SMILES: CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC]

Synthesis of C18:1-SS-K(R)R. C18:1-SS-NH₂ (500 mg, 0.63 mmol) and (Boc-Arg(Pbf))₂-Lys-OH (729 mg, 0.63 mmol) were dissolved in 5 mL DMF and injected into a round bottom flask containing HBTU (288 mg, 0.76 mmol) and HOBT (103 mg, 0.76 mmol) in a nitrogen atmosphere. DIEA (414 mg, 3.2 mmol) was slowly injected into the flask. The reaction was kept at 20° C. for 24 hours with magnetic stirring. The solvent was removed by rotary evaporation. The product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The final product was further purified by column chromatography (MeOH/Ethyl acetate/DCM=1/10/10, v/v/v) on silica gel to give C18:1-SS-Lys-Arg(Pbf))₂-Boc. ¹H NMR (400 MHz, CDCl3): δ 0.88 (t, 6H, terminal methyl group); δ 1.18-1.37 (m, 44H, CH₃(CH₂)₆CH₂CH═CHCH₂(CH₂)₅CH₂—); δ 1.38 (s, 18H, Boc group); δ 1.46 (s, 12H, Pbf group); δ 1.53-1.92 (m, 26H, —CH₂CH₂OCO—, —CH₂CH═CHCH₂, —CH₂CH₂CH₂C(NH—)CO—, —CH₂CH₂C(NH—)CO—); δ 2.09 (s, 6H, Pbf group); δ 2.33-2.49 (m, 10H, —CH₂O(CO)CH₂—, —CH₂N(CH₂—)CH₂—); δ 2.50 (s, 6H, Pbf group); δ 2.57 (s, 6H, Pbf group); δ 2.70-2.83 (m, 6H, —CH₂N(CH₂—)CH₂CH₂SSCH₂CH₂NH—); δ 2.95 (s, 4H, Pbf group); δ 3.09-3.37 (m, 4H, —CH₂OCO—, —CH₂NHC(NH)NH, —CH₂NHCO—); δ 4.05 (dt, 4H, —CH₂CH═CHCH₂—); δ 4.15-4.47 (m, 3H, —CONHCH(CH₂—)CO—).

To expose amine and guanidium groups, C18:1-SS-Lys-Arg(Pbf))₂-Boc (200 mg, 103 μmol) was dissolved in the mixture of 0.5 mL DCM and 0.5 mL TFA and the solution was stirred overnight in a nitrogen atmosphere. The product was concentrated by rotary evaporation and precipitated in diethyl ether to give C18:1-SS-K(R)R.

C18-SS-K(R)R, C14-SS-K(R)R, C18:1-DH-K(R)R, C14-DH-K(R)R were synthesized by reacting the corresponding linker-tail conjugates to (Boc-Arg(Pbf))₂-Lys-OH with the same procedures described in the synthesis of C18:1-SS-K(R)R.

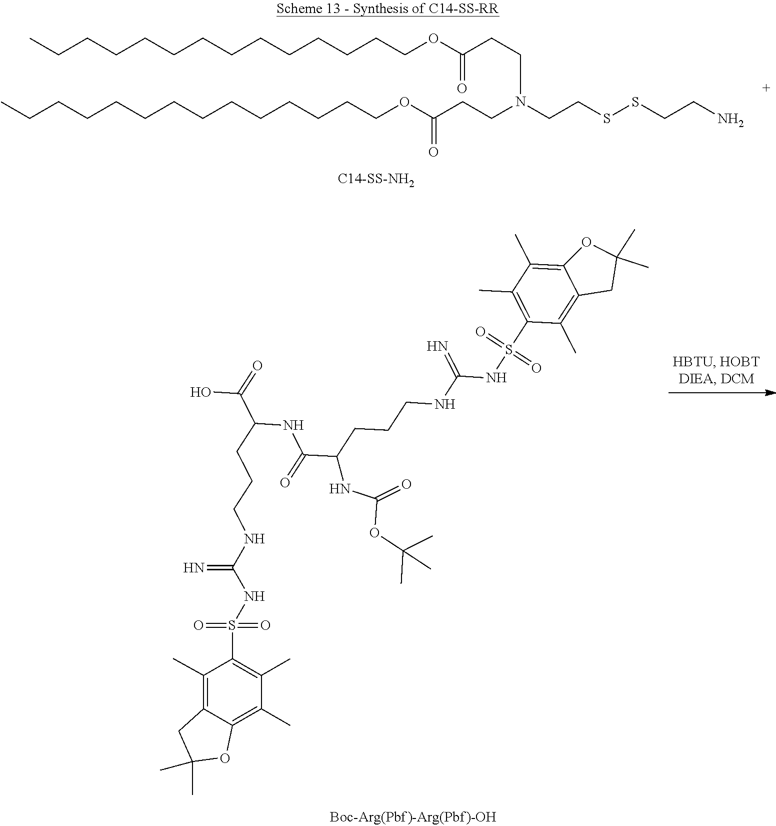

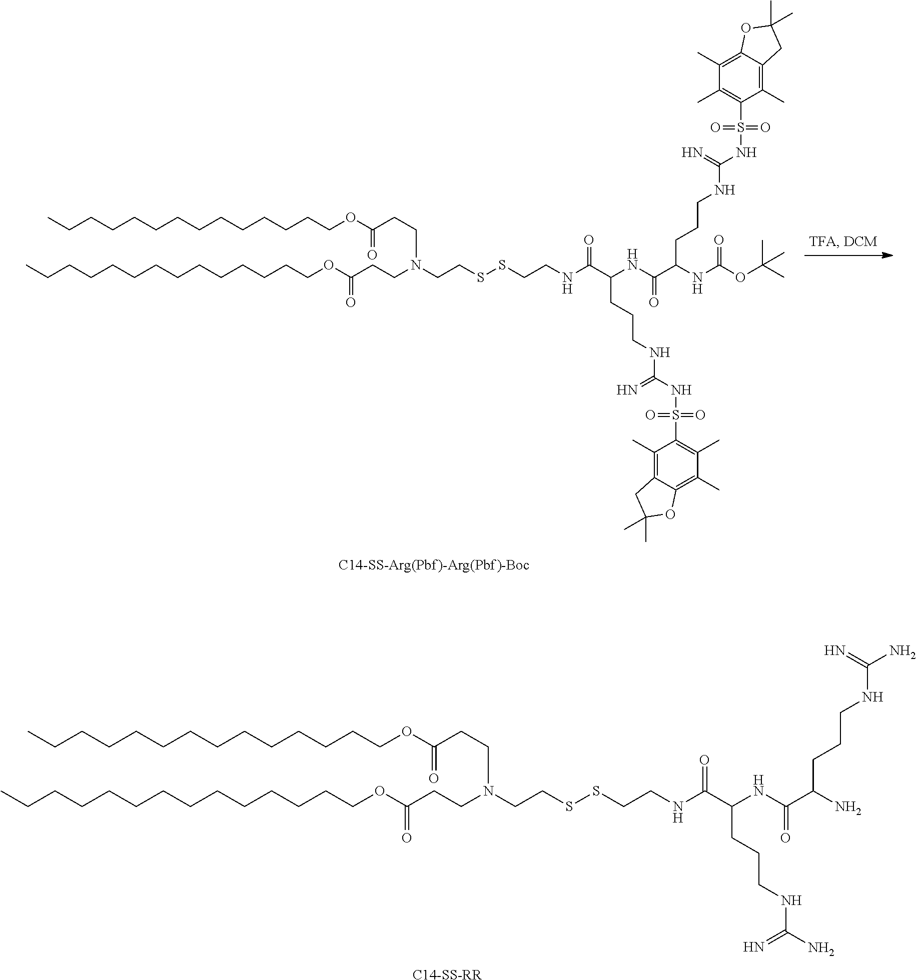

Synthesis of C14-SS-RR. C14-SS-NH₂ (434 mg, 0.63 mmol) and Boc-Arg(Pbf)-Arg(Pbf)-OH (589 mg, 0.63 mmol) were dissolved in 5 mL DMF and injected into a round bottom flask containing HBTU (288 mg, 0.76 mmol) and HOBT (103 mg, 0.76 mmol) in a nitrogen atmosphere. DIEA (414 mg, 3.2 mmol) was slowly injected into the flask. The reaction was kept at 20° C. for 24 hours with magnetic stirring. The solvent was removed by rotary evaporation. The product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The final product was further purified by column chromatography (MeOH/Ethyl acetate/DCM=1/10/10, v/v/v) on silica gel to give C14-SS-Arg(Pbf)-Arg(Pbf)-Boc. ¹H NMR (400 MHz, CDCl₃): δ 0.88 (t, 6H, terminal methyl group); δ 1.18-1.37 (m, 44H, CH₃(CH₂)₆CH₂CH═CHCH₂(CH₂)₅CH₂—); δ 1.38 (s, 9H, Boc group); δ 1.45 (s, 12H, Pbf group); δ 1.53-1.92 (m, 12H, —CH₂CH₂CH₂NH(NH)NH—, —CH₂CH₂OCO—); δ 2.09 (s, 6H, Pbf group); δ 2.33-2.49 (m, 10H, —CH₂O(CO)CH₂—, —CH₂N(CH₂—)CH₂—); δ 2.50 (s, 6H, Pbf group); δ 2.57 (s, 6H, Pbf group); δ 2.95 (s, 4H, Pbf group); δ 3.08-3.29 (m, 4H, —CH₂CH₂CH₂NH(NH)NH—); δ 3.38-3.50 (m, 6H, —CH₂N(CH₂—)CH₂CH₂SSCH₂CH₂NH—); δ 4.08 (t, 4H, —CH₂OCO—); δ 4.25-4.46 (m, 2H, —CONHCH—).

To expose amine and guanidium groups, C14-SS-Arg(Pbf)-Boc (200 mg, 167 μmol) was dissolved in a mixture of 0.5 mL DCM and 0.5 mL TFA and the solution was stirred overnight in a nitrogen atmosphere. The product was concentrated by rotary evaporation and precipitated in diethyl ether to give C14-SS-R.

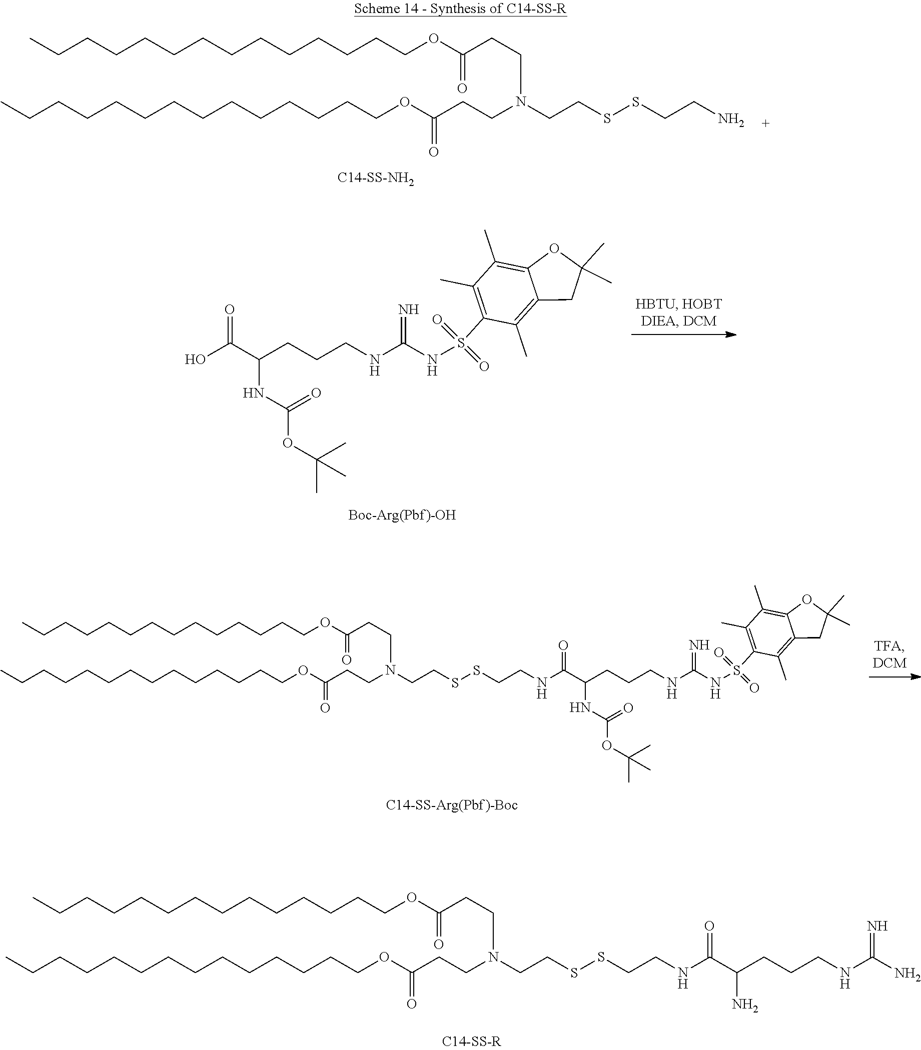

Synthesis of C14-SS-R. C14-SS-NH₂ (434 mg, 0.63 mmol) and Boc-Arg(Pbf)-OH (332 mg, 0.63 mmol) were dissolved in 5 mL DMF and injected into a round bottom flask containing HBTU (288 mg, 0.76 mmol) and HOBT (103 mg, 0.76 mmol) in a nitrogen atmosphere. DIEA (414 mg, 3.2 mmol) was slowly injected into the flask. The reaction was kept at 20° C. for 24 hours with magnetic stirring. The solvent was removed by rotary evaporation. The product was dissolved in 30 mL DCM and washed with saturated NaCl solution, 1 M HCl solution, and saturated NaHCO₃ solution, sequentially. The organic layer was collected and dried by anhydrous MgSO₄ overnight, filtered, and concentrated by rotary evaporation. The final product was further purified by column chromatography (MeOH/Ethyl acetate/DCM=1/10/10, v/v/v) on silica gel to give C14-SS-Arg(Pbf)-Boc. ¹H NMR (400 MHz, CDCl₃): δ 0.88 (t, 6H, terminal methyl group); δ 1.18-1.37 (m, 44H, CH₃(CH₂)₆CH₂CH═CHCH₂(CH₂)₅CH₂—); δ 1.38 (s, 9H, Boc group); δ 1.45 (s, 6H, Pbf group); δ 1.53-1.92 (m, 8H, —CH₂CH₂CH₂NH(NH)NH—, —CH₂CH₂OCO—); δ 2.09 (s, 3H, Pbf group); δ 2.33-2.49 (m, 10H, —CH₂O(CO)CH₂—, —CH₂N(CH₂—)CH₂—); δ 2.50 (s, 3H, Pbf group); δ 2.57 (s, 3H, Pbf group); δ 2.95 (s, 2H, Pbf group); δ 3.08-3.29 (m, 2H, —CH₂CH₂CH₂NH(NH)NH—); δ 4.25-4.46 (m, 1H, —CONHCH—).

To expose amine and guanidium groups, C14-SS-Arg(Pbf)-Boc (200 mg, 167 μmol) was dissolved in the mixture of 0.5 mL DCM and 0.5 mL TFA and the solution was stirred overnight in a nitrogen atmosphere. The product was concentrated by rotary evaporation and precipitated in diethyl ether to give C14-SS-R.

### Example 5—LNP Fabrication Using Additional Ionizable Lipopeptides

RNA-loaded solid lipid nanoparticles (LNP) were fabricated using the ethanol dilution method by microfluidics. Each lipopeptide from Example 4 was dissolved in a mixture of ethanol/THF (1/1, v/v) to obtain a 10 mg/mL stock solution. Each stock solution of lipopeptide was then mixed with DSPE-PEG2000 (90/10, mol/mol) in ethanol to reach a final concentration of 4 mg/mL of total lipids. mRNA was dissolved in 10 mM citrate buffer (pH 4.0) to reach a final concentration of 0.133 mg/mL. The two solutions were rapidly mixed at an aqueous-to-ethanol ratio of 3/1 by volume under a total flow rate of 0.8 mL/min in microfluidics to obtain the LNP termed by the leading lipopeptide. The resulting LNPs were dialyzed (Slide-A-Lyzer Mini Dialysis Units, MWCO 20 kDa) against 1×PBS for 2 h. mRNA concentration was diluted with PBS to 20 ng/μL for in vitro transfection study.

### Characterization

The hydrodynamic diameter and zeta potential of the LNPs were characterized by a dynamic light scattering (DLS) spectrometer (Malvern Zetasizer Nano ZS) as in Example 2.

TABLE 4
The hydrodynamic diameters and Zeta-potentials of LNPs

| Formulation | Size (nm) | Zeta potential (mV) |
|---|---|---|
| AC18:1-SS-K(R)R (GD-LP) | 150.9 | 4.14 |
| C14-SS-K(R)R | 131.7 | 2.21 |
| C18-SS-K(R)R | 149.0 | 4.66 |
| C18:1-SS-K(R)R | 138.4 | 3.12 |
| C14-DH-K(R)R | 144.0 | 2.23 |
| C18:1-DH-K(R)R | 153.6 | 2.29 |
| C14-SS-R | 161.4 | −0.17 |
| C14-SS-RR | 160.9 | 0.22 |

The hydrodynamic diameters of mRNA-loaded LNPs, as measured by dynamic light scattering (DLS), were between 120 to 170 nm (Table 4). Zeta-potential measurements indicated that the mRNA-loaded LNPs had a neutral to slight positive surface charge (Table 4). Compared with nanocomplexes with the liposomal structure formed by AC18:1-SS-K(R)R (described in Examples 1-3), the “solid” LNPs fabricated by microfluidics have a smaller particle size and reduced surface charge that is even more favorable for in vivo applications.

### Example 6—Biological Activity Results for Additional LNPs

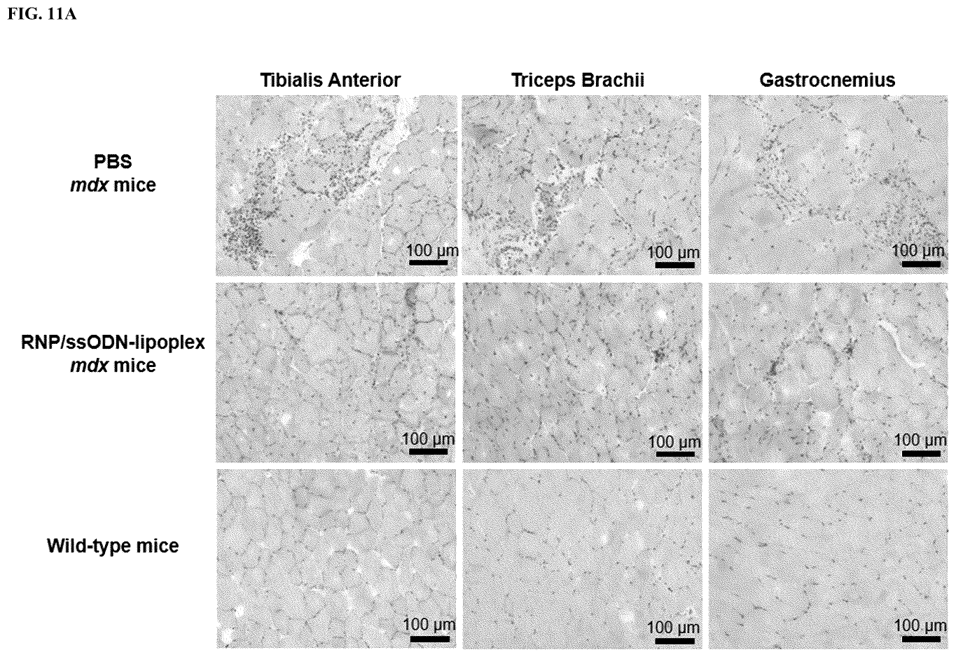

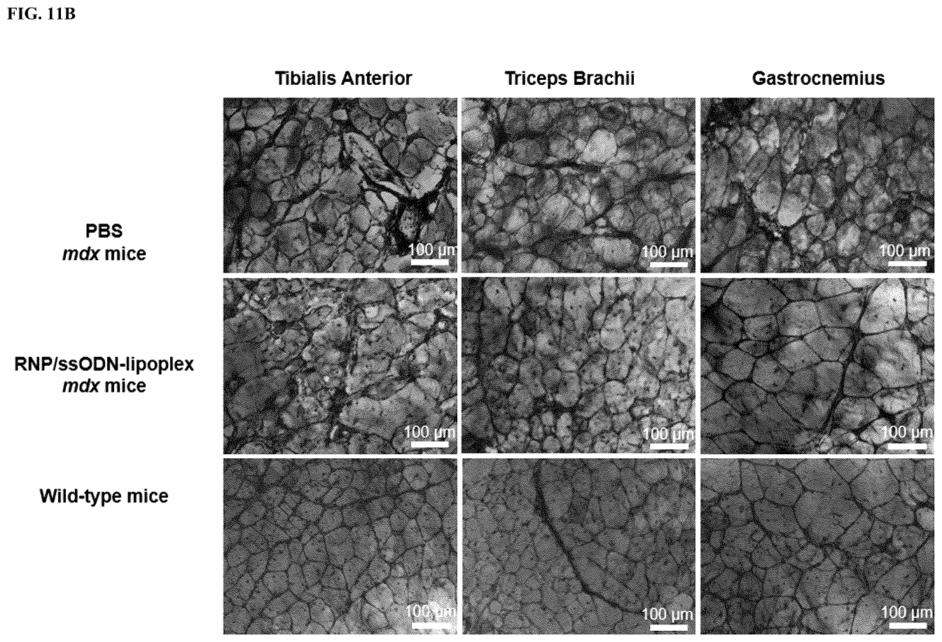

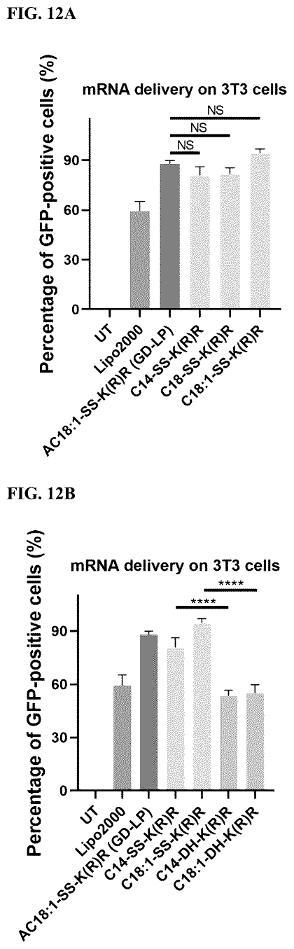

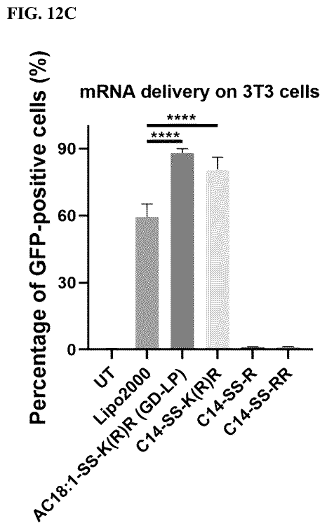

A series of lipopeptide derivatives developed from AC18:1-SS-K(R)R with different tail structures were synthesized. The hydrophobic segments of the new lipopeptides were synthesized by Michael addition between the fatty acrylate and the disulfide linker to simplify the synthetic route. Amide bonds in AC18:1-SS-K(R)R were replaced by ester bonds in new lipopeptides (i.e., C18:1-SS-K(R)R) to enhance biocompatibility. The transfection study showed that the saturation degree and the tail length had minimal impact on the transfection performance (FIG. 12A).

The disulfide linker plays an important role in the lipopeptides. The disulfide bond remains stable in the extracellular glutathione (GSH, 0.001-0.02 mM) and can be degraded in the cytosol where the concentration of GSH is high (2-10 mM), thereby leading to rapid release of the payload mRNA. For instance, LNPs formed by the lipopeptides containing the disulfide bond (C18:1-SS-K(R)R, C14-SS-K(R)R) exhibited a significantly higher transfection efficiency than the lipopeptides without the disulfide linker (C18:1-DH-K(R)R, C14-DH-K(R)R, FIG. 12B), indicating payload release into the cytosol was facilitated by the GSH-triggered disulfide bond degradation.

The oligopeptide for the head structure is another critical factor for the lipopeptide, as the head structure offers the positive charge for mRNA binding via electrostatic interaction and helps LNPs to penetrate the cell membrane through the bidentate hydrogen bonds formation between guanidium groups and cell-surface anions. The replacement of the Y-shape lysine-arginine oligopeptide by single arginine (C14-SS-R) or arginine dipeptide (C14-SS-RR) eliminated the transfection ability of the lipopeptides (FIG. 12C), indicating the Y-shape lysine-arginine oligopeptide is the key factor for the transfection performance of this series of lipopeptides.

### Example 7—LNP Fabrication Using Helper Lipids

Two four-lipid formulations of LNPs of the present technology were prepared. Each lipopeptide (shown in Table 2) was mixed with helper lipids (cholesterol, 24α-ethyl-cholesterol, and distearoylphosphatidylcholine (DSPC)) in ethanol at a mol/mol ratio of 50/10/38.5/1.5 lipopeptide/DSPC/Cholesterol/DSPE-PEG2000, to reach a final concentration of 4 mg/mL of the total lipids. mRNA was dissolved in 10 mM citrate buffer (pH 4.0) to reach a final concentration of 0.133 mg/mL. The two solutions were rapidly mixed at an aqueous-to-ethanol ratio of 3/1 by volume under a total flow rate of 0.8 mL/min in microfluidics to obtain the LNP termed by the leading lipopeptide. The resulting LNPs were dialyzed (Slide-A-Lyzer Mini Dialysis Units, MWCO 20 kDa) against 1×PBS for 2 h. mRNA concentration was diluted with PBS to 20 ng/μL for in vitro transfection studies.

The hydrodynamic diameter and zeta potential of the LNPs were characterized as in Example 5. Results are shown in Table 2. With the helper lipids, the lipopeptide C14-SS-K(R)R successfully formed LNPs (termed C14-SS-K(R)R_chol) by microfluidics. 24α-Ethyl cholesterol, a cholesterol derivative reported to help the endosomal escape, was also used to fabricate an LNP (termed C14-SS-K(R)R_24α-ethyl-chol) of the present technology. These two types of LNPs had similar sizes (about 150-170 nm) with a slightly positive charge (7-10 mV, Table 2).

TABLE 2
The hydrodynamic diameters and Zeta-potentials of
the four-lipid LNP formulations by microfluidics.

| | | | Zeta |
|---|---|---|---|
| | Formulation | Size | potential |
| Name | (molar ratio) | (nm) | (mV) |
| C14-SS- | C14-SS-K(R)R/DSPC/Chol/DSPE- | 162.3 | 7.23 |
| K(R)R_chol | PEG = 50/10/38.5/1.5 | | |
| C14-SS- | C14-SS-K(R)R/DSPC/24α-ethyl- | 151.7 | 9.67 |
| K(R)R_24α- | Chol/DSPE-PEG = 50/10/38.5/1.5 | | |
| ethyl-Chol | | | |

### Example 8—Biological Activity Results for LNPs Formulated with Helper Lipids

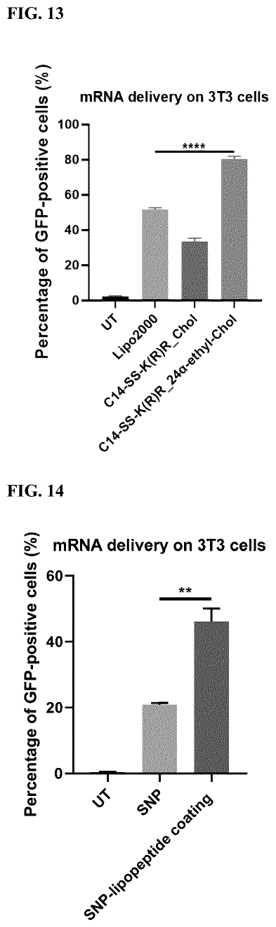

LNP formulations fabricated with the addition of cholesterol and DSPC (i.e., (lipopeptide/DSPC/Cholesterol/DSPE-PEG2000, 50/10/38.5/1.5 mol/mol)) were also evaluated for transfection stability. An in vitro transfection study carried out as above in NIH 3T3 cells showed a high transfection efficiency of 80.48% in the C14-SS-K(R)R_24α-ethyl-chol treated group (FIG. 13). With the integration of 24α-ethyl cholesterol and DSPC, the LNP formed by C14-SS-K(R)R possesses better stability, while exhibiting a comparable transfection performance with the original lipopeptide/DSPE-PEG LNP formulations, making it more attractive for intravenous injection applications.

### Example 9—Fabrication and Biological Activity of Lipopeptide-Coated Silica Nanocapsule

A lipopeptide of the present technology was tested as a surface coating on a silica nanoparticle (SNP) to determine whether altering the surface chemistry could further enhance delivery efficiency by the SNP. For this study, mRNA-loaded SNP was synthesized by a water-in-oil microemulsion method as described in WO 2021/23662. Briefly, 6 μL bis(triethoxysilylpropyl) disulfide, 4 μL triethoxyvinylsilane, and 30 uL GFP-mRNA stock solution (1 mg/mL) were homogenized by pipetting and added to stirred 1 mL organic phase (cyclohexene/Triton-X/hexanol, 7.5/1.75/1.75, vol/vol/vol). Ammonium hydroxide solution (2 μL, 30% NH₃) was added under vigorous stirring (1,500 rpm) to initiate the reaction and the water-in-oil microemulsion was allowed to stir at 20° C. for 4 hours. The reaction mixture was precipitated by acetone and washed with ethanol twice. The purified mRNA-loaded SNP was finally collected by centrifugation and dispersed in ultrapure water to get an SNP solution with an mRNA concentration of 0.1 mg/mL. mRNA-loaded SNP was then mixed with Empty-LNP (mRNA/LNP, 1/10, w/w) prepared as in Example 2 above and was passed through an extrusion membrane (0.2 μm) to yield lipopeptide-coated SNP.

The lipopeptide-coated SNP was tested for efficiency of transfection as described in the general procedures. As shown in FIG. 14, the lipopeptide-coated SNP enhanced the mRNA delivery efficiency in NIH 3T3 cells by about 120%.

### Example 10—Lipopeptide-Based LNPs Enable Efficient Gene Editing in Retinal Pigment Epithelium

Fabrication of LNPs with helper lipids for gene delivery. The lipopeptide was mixed with helper lipids in methanol at the desired molar ratio, achieving a final concentration of 30 mg/mL of total lipids. The mRNA or plasmid was dispersed in 10 mM citrate buffer at pH 5.0, and the ribonucleoprotein (RNP) in 10 mM Tris-HCl buffer at pH 7.4, both to a concentration of 1 mg/mL. These aqueous solutions were then rapidly mixed with the methanol lipid solution at a 3:1 aqueous-to-methanol ratio using pipetting and vortexing to form the lipid nanoparticles (LNPs). The LNPs were dialyzed (Slide-A-Lyzer Mini Dialysis Units, MWCO 20 kDa) against 1×PBS for 2 h and were then ready for in vivo study. For in vitro studies, the LNPs were diluted to 20 ng/μL with 1×PBS, according to the payload concentration.

Optimization of LNP formulations. To optimize the buffer pH for mRNA encapsulation in the fabrication of LNPs, GFP mRNA was dispersed in 10 mM citrate buffer at various pH levels (pH 3, pH 5 and pH 6), each adjusted using 1 M NaOH, or in 1×PBS at pH7. For the lipid composition optimization of LNPs, we varied the molar percentage of the lipid AC18:1-SS-K(R)R from 50% to 98.5%. The molar ratio of DMG-PEG₂ₖ was fixed at 1.5%. The molar ratio of DSPC to 24α-ethyl cholesterol was maintained at 10:38.5. For LNP formulation with a 98.5% molar ratio of lipopeptide, DSPC and 24α-ethyl cholesterol were not used.

Lyophilization of LNPs. Freshly prepared LNPs with an initial payload concentration of 0.75 mg/mL were dialyzed against PBS at 1×, 0.5×, or 0.25×, with the pH adjusted to 7.4 using 1M NaOH solution. Prior to freeze-drying, the LNPs were mixed with an equal volume of freeze-drying buffer (20 mM Tris-HCl at pH 7.4 in either a 10% or 5% sucrose solution). The LNPs were first flash-frozen in liquid nitrogen, and then lyophilization was carried out using a FreeZone Triad Benchtop Freeze Dryer (LABCONCO) set to a −55° C. cycle for 48 hours. The lyophilized powder was subsequently stored at 4° C. Post-lyophilization, LNPs were resuspended in DI water to concentrations ranging from 0.5 to 3 mg/mL.

In vitro CRISPR/Cas9 Genome Editing Efficiency Study. GFP-HEK 293 cells were seeded at a density of 5,000 cells per well in 96-well plates 24 hours before treatment. The cells were treated with LNPs loaded with Cas9 mRNA/sgRNA or RNP at doses of 50 ng/well and 125 ng/well, respectively. Two days post-treatment, an additional 50 μL of fresh culture medium was supplemented to each well. Cells were harvested for analysis four days post-treatment. Editing efficiencies were quantified using a flow cytometer and the data were analyzed by FlowJo v7.6.

Subretinal injection. A fixed volume of 2 μL of the LNP solution was injected into the subretinal space using a UMP3 ultramicro pump, a NanoFil syringe, and an RPE-KIT that included a 34-gauge beveled needle, all from World Precision Instruments. The tip of the needle remained in the formed bleb for 10 s before gentle withdrawal. Mice were euthanized 7 days after Cre-mRNA delivery or 14 days after CRISPR genome editing, and eyes were collected for tdTomato expression assessment. The collected eyes were rinsed twice with PBS, punctured at the ora serrata with an 18-gauge needle, and then opened along corneal incisions. The eyecup was incised radially to the center and flattened into a floret shape, after which the RPE layer was separated and flat-mounted on a cover glass slide for imaging (i.e., RPE floret). The RPE florets were imaged using a Nikon C2 confocal laser scanning microscope (CLSM) with NIS-Elements software.

Immunofluorescence staining for CLSM analysis. Eyeballs were fixed in 4% paraformaldehyde (PFA) at 4° C. for 24 hours and then incubated overnight in 1×PBS containing 30% sucrose at 4° C. for dehydration. Tissues were then embedded in Tissue-Tek Optimal Cutting Temperature (OCT) Compound (Sakura Finetek) and frozen in dry ice. Blocks were sectioned at a thickness of 10 m and mounted on microscope slides. Retinal cryosections were washed three times with 1×PBS, followed by 1 hour incubation in 1×PBS with 10% goat serum and 0.3% Triton X-100 for permeabilization. Primary antibodies, Rabbit anti-RFP (ab152123, 1:1,000) and Mouse anti-visual arrestin E-3 (Cat. #SC-166383, 1:100), were applied in PBS containing 10% goat serum and incubated overnight at 4° C. After primary antibody incubation, sections were washed thrice in 1×PBS with 0.3% Tween20, then incubated with secondary antibodies-Anti-Rabbit IgG H&L (Alexa Fluor® 594, ab150080, 1:1,000) and Anti-Mouse IgG H&L (Alexa Fluor® 488, ab150113, 1:1,000)—in PBS with 10% goat serum for 1 hour at room temperature. Sections were counterstained with DAPI for 5 minutes at room temperature, mounted with ProLong Gold Antifade Reagent, and covered with cover glasses. Imaging was performed using CLSM.

### Results

### Preparation of Lipopeptide-Based Solid Lipid Nanoparticles

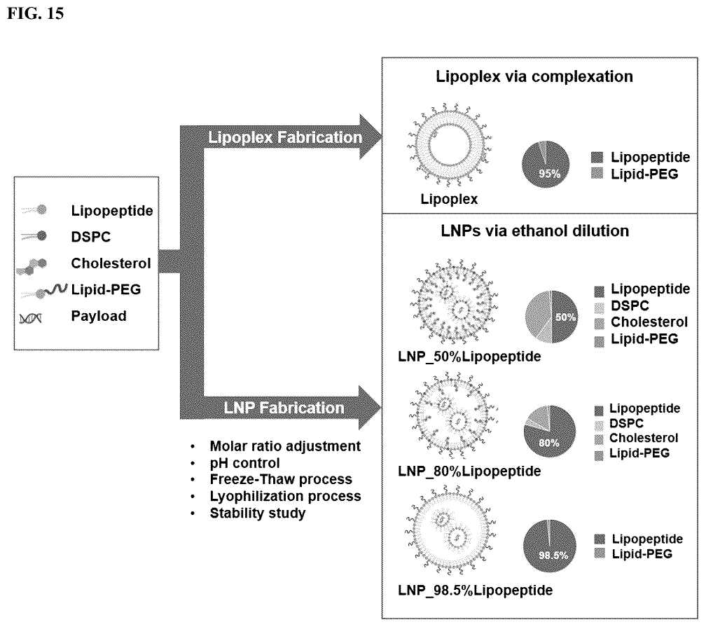

Solid lipid nanoparticles (solid LNPs or just LNPs in the present example) using varying amounts of lipopeptides were prepared as shown schematically in FIG. 15. Briefly, the lipopeptide and helper lipids were dissolved in methanol as the organic phase. The payload consisting of nucleic acids (e.g., mRNA or DNA) or CRISPR genome editors (e.g., Cas9 mRNA/sgRNA or RNP) or base editors (e.g., ABE8e-mRNA/sgRNA) was prepared in citrate buffer or Tris HCl buffer as the aqueous phase. The two phases were rapidly mixed by pipetting and vortexing. For large-scale fabrication, a microfluidic staggered herringbone mixer was used for mixing².

Optimization of Solid LNPs for DNA and mRNA Delivery

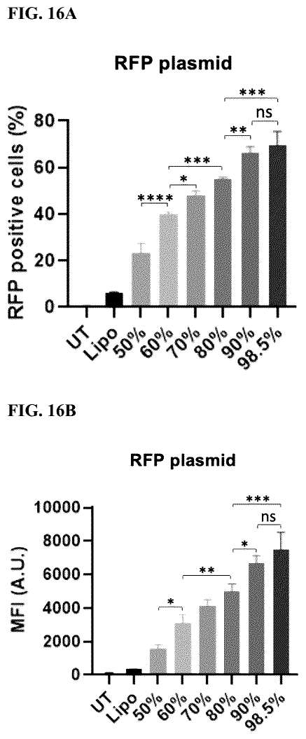

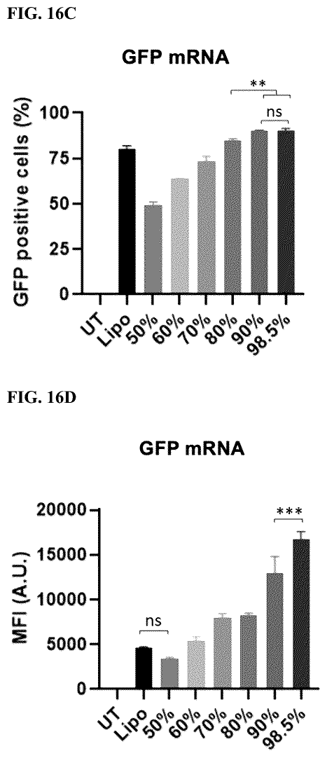

The transfection efficiencies of the solid LNPs were evaluated in NIH 3T3 cells. Based on the most common lipid formulation (i.e., 50% ionizable lipid, 38.5% cholesterol, 10% DSPC, and 1.5% DMG-PEG₂ₖ, molar ratios) in commercialized products (e.g., COVID-19 mRNA vaccines from Moderna and Pfizer/BioNTech), the molar ratio of the lipopeptide in the solid LNP was optimized by fine-tuning the amount of lipopeptide AC18:1-SS-K(R)R and keeping other components to the same ratio (termed X % AC18:1-SS-K(R)R LNP, X is the molar ratio of the lipopeptide) The in vitro transfection efficiency was enhanced by increasing the molar ratio of lipopeptide from 50% to 98.5%. In particular, solid LNP with no DSPC and cholesterol (molar ratio of 98.5%) showed a similar mRNA transfection efficiency, but higher mean fluorescence intensity (MFI) than 90% AC18:1-SS-K(R)R LNP as shown in FIG. 16D. The MFI of the GFP signal also demonstrated that the higher molar ratio of lipopeptide led to higher mRNA transfection performance (FIG. 16C). The same phenomena were observed in the plasmid encapsulated solid LNP as shown in FIG. 16A and FIG. 16B. This is because the AC18:1-SS-K(R)R lipopeptide is the key component in the solid LNP formulation. The guanidium group on the lipopeptide was almost completely ionized as a cation due to its high pKa of 13.8, and therefore has sufficient charge to complex with anionic mRNA or DNA without the assistance of other helper lipids. Increased lipopeptide amounts can lead to higher surface charge density which will facilitate the cellular uptake and endosomal escape of the nanoparticles, thereby enhancing the transfection efficiency. The hydrodynamic sizes of the screened solid LNPs (Table 5) indicated a trend of decreasing particle size with an increasing molar ratio of AC18:1-SS-K(R)R lipopeptide.

TABLE 5
Hydrodynamic sizes of the solid LNPs based on the lipopeptide
AC18:1-SS-K(R)R encapsulated with RFP plasmid or GFP mRNA.

| | | Molar ratio | | | | DLS |
|---|---|---|---|---|---|---|
| # | | LP | DSPC | 24α-chol | C14-PEG | Size(nm) |
| RFP plasmid | | | | | | |
| 1 | pH 5 | 50 | 10.00 | 38.50 | 1.5 | 209.1 ± 2.8 |
| 2 | pH 5 | 60 | 7.94 | 30.56 | 1.5 | 193.1 ± 3.2 |
| 3 | pH 5 | 70 | 5.88 | 22.62 | 1.5 | 160.0 ± 2.7 |
| 4 | pH 5 | 80 | 3.81 | 14.69 | 1.5 | 150.0 ± 1.7 |
| 5 | pH 5 | 90 | 1.75 | 6.75 | 1.5 | 125.8 ± 1.6 |
| 6 | pH 5 | 98.5 | 0 | 0 | 1.5 | 122.9 ± 2.2 |
| GFP mRNA | | | | | | |
| 1 | pH 5 | 50 | 10.00 | 38.50 | 1.5 | 184.1 ± 4.0 |
| 2 | pH 5 | 60 | 7.94 | 30.56 | 1.5 | 165.0 ± 1.6 |
| 3 | pH 5 | 70 | 5.88 | 22.62 | 1.5 | 152.9 ± 3.3 |
| 4 | pH 5 | 80 | 3.81 | 14.69 | 1.5 | 149.7 ± 2.4 |
| 5 | pH 5 | 90 | 1.75 | 6.75 | 1.5 | 134.8 ± 1.6 |
| 6 | pH 5 | 98.5 | 0 | 0 | 1.5 | 136.4 ± 4.1 |

Optimization of Solid LNPs for Cas9 mRNA/sgRNA and RNP Delivery

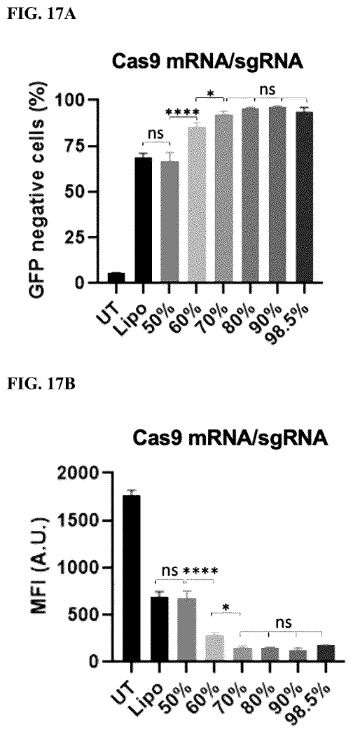

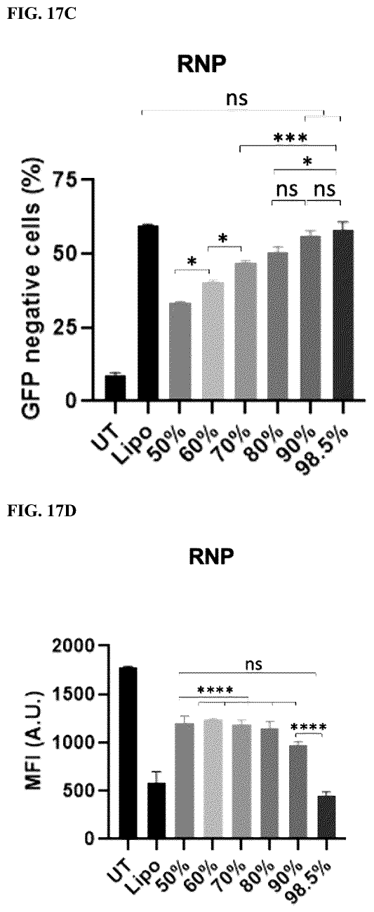

Cas9 gene editing mediated by the lipopeptide based solid LNP was studied. The solid LNPs with different molar ratios of lipopeptide were fabricated to encapsulate Cas9 mRNA/sgRNA or Cas9 RNP. The resulting solid LNPs were tested on GFP-HEK cells. Successful delivery of RNP results in GFP gene knockdown and subsequently suppress GFP expression, which can be quantified by flow cytometry. For Cas9 mRNA/sgRNA delivery (FIG. 17A and FIG. 17B), increasing the lipopeptide molar ratio from 50% to 70% enhanced GFP knockout efficiency. Solid LNP with a molar ratio above 70% achieved up to 95% GFP knockout, suggesting a potential efficiency plateau. For RNP delivery (FIG. 17C and FIG. 17D), editing efficiency peaked at 60% as the lipopeptide molar ratio increased to 98.5%. The hydrodynamic sizes of the solid LNPs for both Cas9 mRNA/sgRNA and RNP delivery (Table 6) decreased with an increasing molar ratio of AC18:1-SS-K(R)R lipopeptide, a similar trend observed with the solid LNPs encapsulating DNA and mRNA.

TABLE 6
Hydrodynamic sizes of the solid LNPs based
on the lipopeptide AC18:1-SS-K(R)R encapsulated
with Cas9 mRNA/sgRNA or Cas9 RNP.

| | | Molar ratio | | | | |
|---|---|---|---|---|---|---|
| | | | | 24α- | | DLS |
| # | | LP | DSPC | chol | C14-PEG | Size(nm) |
| Cas9 mRNA/sgRNA | | | | | | |
| 1 | pH 5 | 50 | 10.00 | 38.50 | 1.5 | 212.0 ± 3.7 |
| 2 | pH 5 | 60 | 7.94 | 30.56 | 1.5 | 182.6 ± 1.6 |
| 3 | pH 5 | 70 | 5.88 | 22.62 | 1.5 | 153.2 ± 2.1 |
| 4 | pH 5 | 80 | 3.81 | 14.69 | 1.5 | 146.0 ± 2.1 |
| 5 | pH 5 | 90 | 1.75 | 6.75 | 1.5 | 133.9 ± 3.7 |
| 6 | pH 5 | 98.5 | 0 | 0 | 1.5 | 128.3 ± 2.3 |
| RNP | | | | | | |
| 1 | pH 7.5 | 50 | 10.00 | 38.50 | 1.5 | 184.2 ± 4.6 |
| 2 | pH 7.5 | 60 | 7.94 | 30.56 | 1.5 | 182.0 ± 8.8 |
| 3 | pH 7.5 | 70 | 5.88 | 22.62 | 1.5 | 116.4 ± 9.3 |
| 4 | pH 7.5 | 80 | 3.81 | 14.69 | 1.5 | 128.0 ± 3.4 |
| 5 | pH 7.5 | 90 | 1.75 | 6.75 | 1.5 | 119.2 ± 4.2 |
| 6 | pH 7.5 | 98.5 | 0 | 0 | 1.5 | 113.3 ± 6.2 |

Optimization of Buffer pH for mRNA Encapsulation

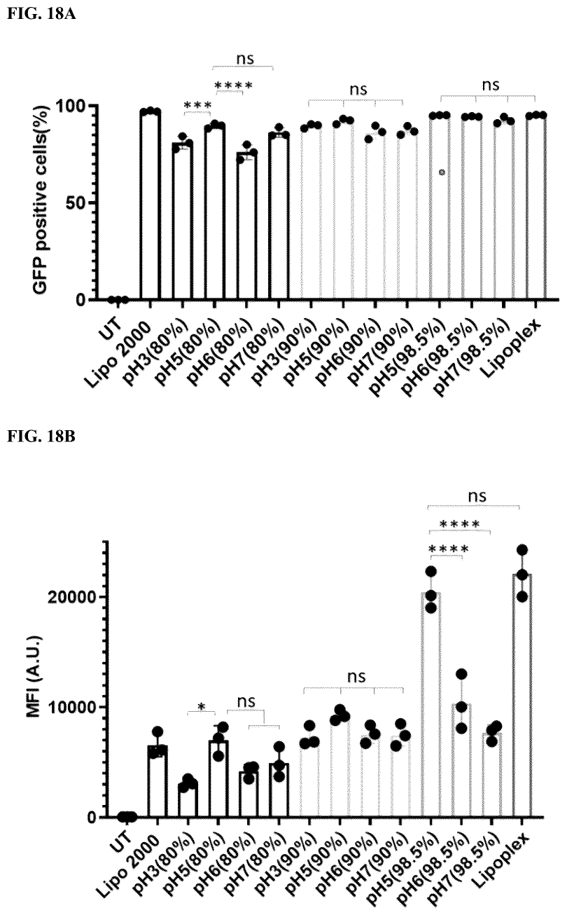

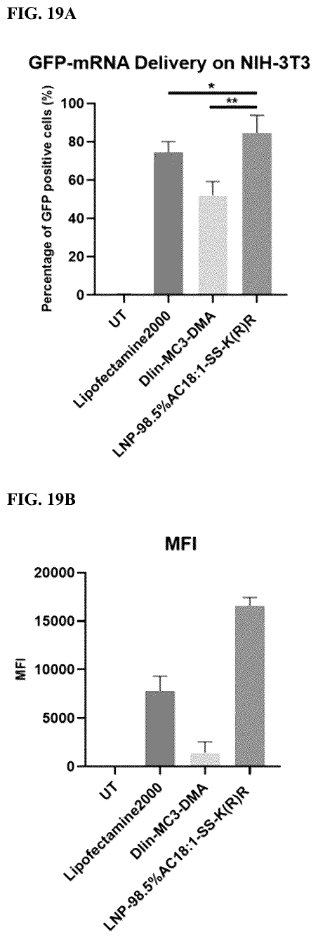

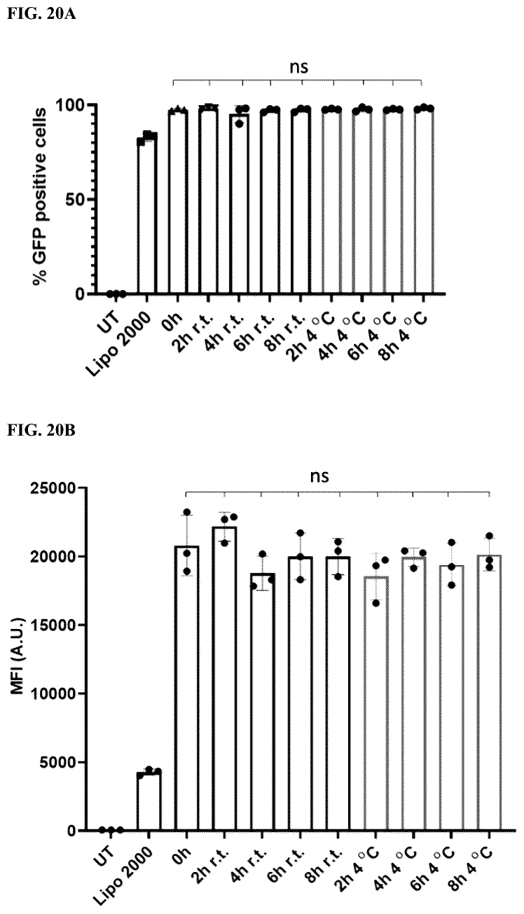

We optimized the pH of the citrate buffer used to form the nanoparticles. The in vitro transfection study demonstrated that a pH of 5 was the optimal value for the citrate buffer solution (FIGS. 18A-18B). There were minimal changes in the hydrodynamic sizes of the solid LNPs at different pH levels (Table 7). As discussed above, the ionization of the guanidium group does not require a low pH for the fabrication of mRNA-loaded solid LNPs. Moreover, a low pH citrate buffer may damage the fragile mRNA. Therefore, a higher pH resulted in more effective complexation of the guanidium-rich lipopeptide with mRNA, compared to the traditional ionizable solid LNP formulations which mostly use an mRNA solution at pH 3 or 4. Notably, the selected formulation 98.5% AC18:1-SS-K(R)R LNP at pH 5 exhibited higher transfection efficiency and significantly stronger MFI of the GFP signal for mRNA transfection in NIH 3T3 cells, compared with the commercial solid LNP fabricated by Dlin-MC3-DMA (FIGS. 19A-19B). Additionally, the 98.5% AC18:1-SS-K(R)R LNP formulation displayed excellent stability, with hydrodynamic sizes remaining unchanged (Table 8) and transfection performance consistent after storage for at least 8 hours at both room temperature and 4° C. (FIGS. 20A-20B).

TABLE 7
Hydrodynamic sizes of the solid LNPs based on the lipopeptide
AC18:1-SS-K(R)R fabricated with different mRNA buffer pH.

| # | | LP | Size(nm) |
|---|---|---|---|
| 1 | pH 3 | 80 | 121.6 ± 4.2 |
| 2 | pH 5 | 80 | 121.3 ± 3.8 |
| 3 | pH 6 | 80 | 122.0 ± 2.7 |
| 4 | pH 7 | 80 | 134.6 ± 2.3 |
| 5 | pH 3 | 90 | 90.3 ± 3.1 |
| 6 | pH 5 | 90 | 126.9 ± 2.9 |
| 7 | pH 6 | 90 | 130.1 ± 5.1 |
| 8 | pH 7 | 90 | 124.5 ± 3.7 |
| 9 | pH 5 | 98.5 | 131.8 ± 4.5 |
| 10 | pH 6 | 98.5 | 125.3 ± 1.4 |
| 11 | pH 7 | 98.5 | 133.9 ± 2.8 |
| 15 | Lipoplex | | 250.9 ± 7.2 |

TABLE 8
Stability of the 98.5% AC18:1-SS-K(R)R solid LNPs stored
at room temperature or 4° C. determined by DLS.

| # | Temperature | Stored time | Size(nm) |
|---|---|---|---|
| 1 | | 0 h | 128.3 ± 5.7 |
| 2 | r.t. | 2 h | 128.7 ± 5.2 |
| 3 | | 4 h | 128.1 ± 5.3 |
| 4 | | 6 h | 131.8 ± 2.3 |
| 5 | | 8 h | 133.2 ± 6.0 |
| 6 | 4° C. | 2 h | 130.7 ± 3.1 |
| 7 | | 4 h | 131.9 ± 3.5 |
| 8 | | 6 h | 129.0 ± 2.0 |
| 9 | | 8 h | 127.7 ± 4.4 |

### Optimization of Lyophilized Solid LNPs for Enhanced Transfection Efficiency and Stability

The feasibility of lyophilized solid LNPs, specifically the 98.5% AC18:1-SS-K(R)R LNP formulation, was explored. To preserve the osmotic balance of solid LNP solutions after lyophilization, fresh solid LNPs were dialyzed against PBS at various concentrations (1×, 0.5×, and 0.25×). Subsequently, the solid LNPs were supplemented with sucrose at different concentrations (5% and 10%) as a cytoprotectant prior to lyophilization. The lyophilized solid LNPs were redispersed in variable volumes of DI water to obtain different solid LNP concentrations. As shown in Table 9, the particle sizes of fresh solid LNPs ranged from 120 to 130 nm, indicating the dialysis buffer concentration minimally influenced particle sizes. The hydrodynamic sizes of lyophilized solid LNPs mostly increased to 160-165 nm when the concentration was doubled from 0.75 mg/mL to 1.50 mg/mL, and the resulting particles remained stable for 6 hours. However, redispersing lyophilized solid LNPs at a higher concentration (3.00 mg/mL) and storing for over 8 hours at 4° C. led to aggregation of solid LNPs, with particle sizes increasing to approximately 190 nm.

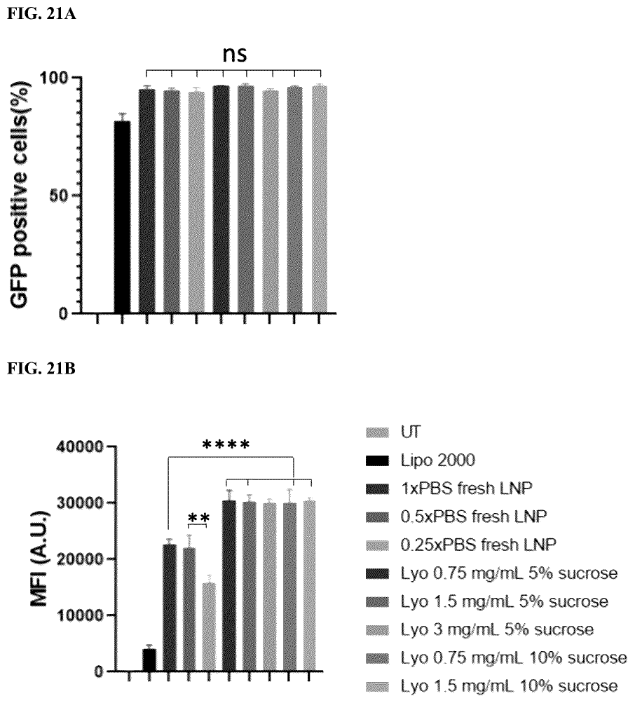

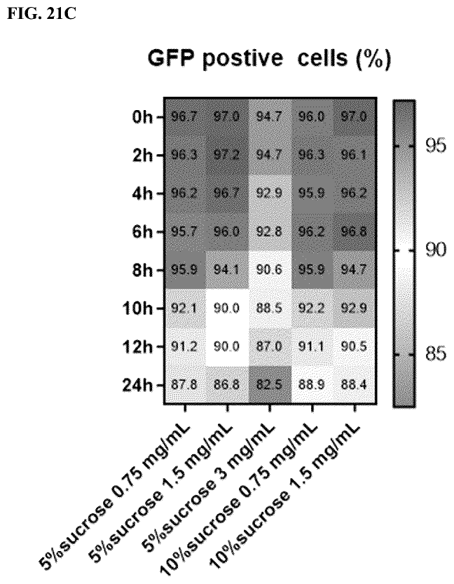

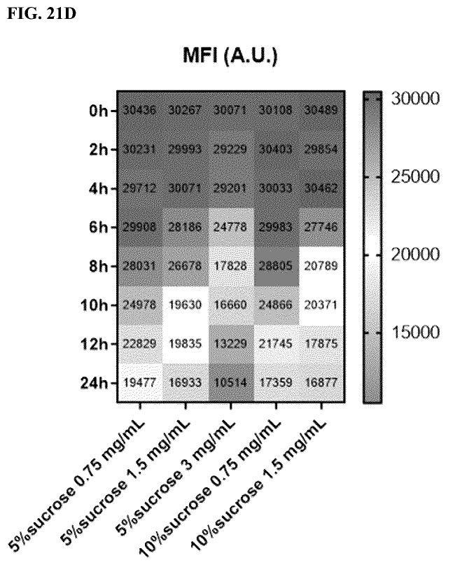

Transfection efficiency was compared between fresh and lyophilized solid LNPs in NIH 3T3 cells. As depicted in FIG. 21A and FIG. 21B, no significant difference was observed in the percentage of transfected cells, although the MFI was significantly higher with lyophilized solid LNPs. Notably, solid LNPs dialyzed in 0.25×PBS exhibited a significant reduction in MFI compared to those dialyzed in higher-concentration PBS, underscoring the importance of ionic strength in the dialysis buffer for effective mRNA encapsulation. The stability of lyophilized solid LNPs reconstituted in DI water for various durations, was also assessed. Solid LNPs were resuspended at concentrations of 0.75 mg/mL and 1.50 mg/mL maintained high transfection efficiency (FIG. 21C) and MFI (FIG. 21D) when stored for up to 6 hours, aligning with the size measurement data in Table 9 showing particle sizes remained unchanged for same duration. The stabilization of 1.5 mg/mL lyophilized solid LNPs was not significantly impacted by the sucrose concentration in the freeze-drying buffer, with both 5% and 10% sucrose showing comparable results. This finding is advantageous for future in vivo applications since a lower 5% sucrose concentration is preferred to reduce the risk of potential tissue dehydration damage associated with higher levels of sucrose. In summary, the lyophilization process of the present solid LNP system was validated, enhancing dosing capabilities for volume-restricted administrations, such as the subretinal injection.

TABLE 9
Hydrodynamic sizes of the 98.5% AC18:1-SS-K(R)R solid LNPs dialyzed in
the diluted PBS and the stability of the lyophilized solid LNPs at 4° C.

| | Size(nm) | | |
|---|---|---|---|
| Dialysis Buffer | 1x PBS | 0.5x PBS | 0.25x PBS |
| Fresh 98.5% | 127.2 ± 3.0 | 128.1 ± 2.5 | 130.3 ± 3.7 |
| solid LNP | | | |

| | mRNA con. | Size(nm) | | | | |
|---|---|---|---|---|---|---|
| | (mg/mL) | 0 h | 2 h | 4 h | 6 h | 8 h |
| 5% | 0.75 | 165.0 ± 3.7 | 164.0 ± 4.7 | 163.0 ± 3.0 | 166.6 ± 3.1 | 163.0 ± 4.7 |
| sucrose | 1.5 | 165.6 ± 2.3 | 162.5 ± 6.6 | 162.4 ± 4.5 | 164.8 ± 6.3 | 183.6 ± 7.8 |
| | 3 | 160.9 ± 5.9 | 185.6 ± 8.7 | 186.7 ± 11.6 | 189.7 ± 4.9 | 191.7 ± 4.0 |
| 10% | 0.75 | 161.6 ± 3.5 | 163.7 ± 5.4 | 163.7 ± 5.4 | 166.3 ± 2.6 | 188.8 ± 9.5 |
| sucrose | 1.5 | 161.0 ± 1.3 | 163.2 ± 5.8 | 164.6 ± 7.3 | 163.7 ± 1.3 | 184.0 ± 8.7 |

### In Vivo Genome Editing by Solid LNPs in Ai14 Mice

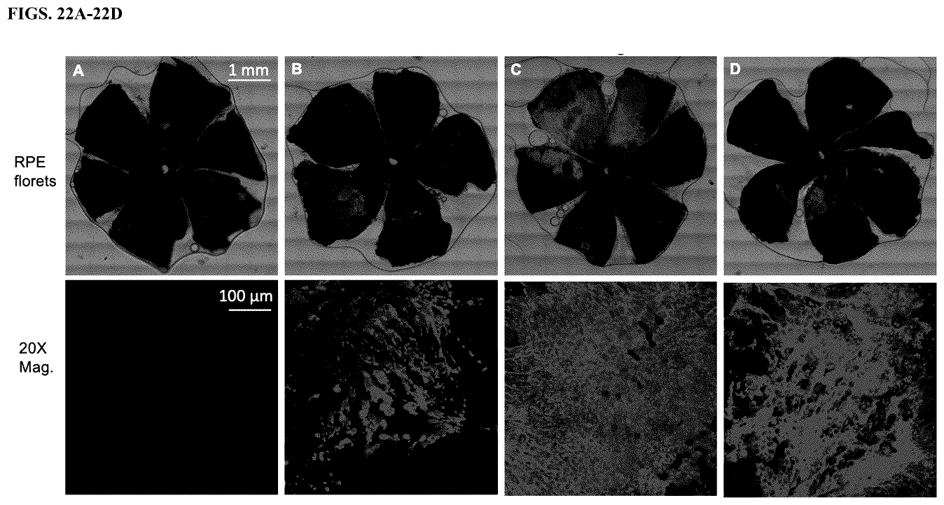

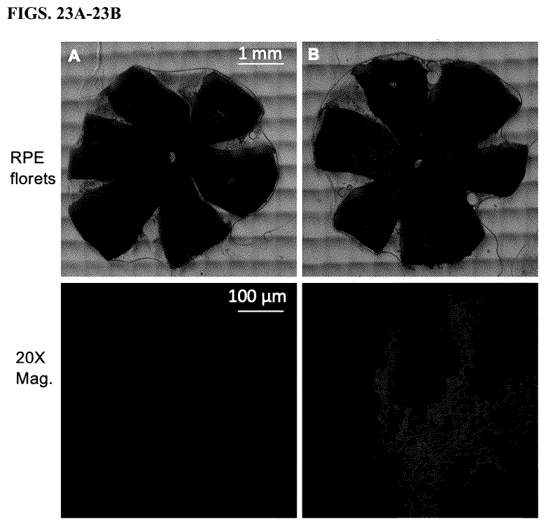

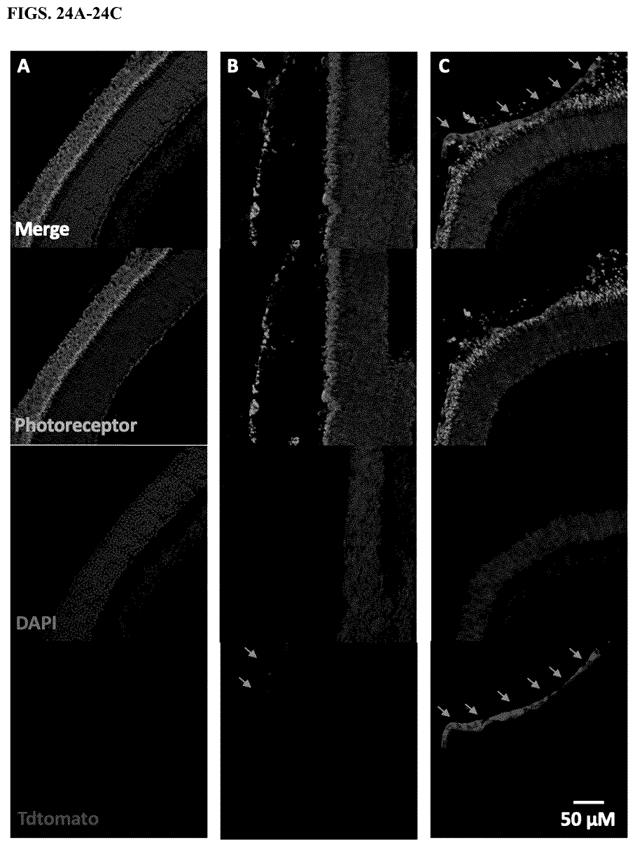

After confirming the efficacy of solid LNPs for in vitro delivery of nucleic acids and genome editors etc., we investigated the in vivo gene editing efficiency of the solid LNPs using the Ai14 transgenic genome editing reporter mouse model. In Ai14 mice, a LoxP-flanked STOP cassette prevents the transcription of tdTomato reporter protein, regulated by a CAG promoter. Cre recombinase-mediated recombination can remove this STOP cassette, enabling tdTomato expression as a fluorescent marker for gene editing efficiency evaluation. For in vivo Cre mRNA delivery via subretinal injection in Ai14 mice, the 98.5% AC18:1-SS-K(R)R solid LNP and Lipoplex were selected due to its superior in vitro delivery results. Additionally, we employed 90.5% AC18:1-SS-K(R)R/8% PS solid LNP, incorporating an 8% molar ratio of DOPS, to boost Cre mRNA delivery efficacy in RPE cells. This strategy of incorporating DOPS exploits the natural translocation of phosphatidylserine (PS) from the inner to the outer cellular membrane during apoptosis or the shedding of photoreceptor outer segments (POS), which acts as a signal for RPE cells to phagocytose the affected cells or POS³. Mice were euthanized seven days post-injection, and the RPE florets were isolated and imaged by the confocal laser scanning microscopy. Compared to the PBS group (FIG. 22A), all three formulations achieved relatively widespread tdTomato expression with strong florescence intensity (FIGS. 22B-22D). Notably, the 90.5% AC18:1-SS-K(R)R/8% PS solid LNP induced more pronounced tdTomato expression, attributed to the incorporation of PS enhancing RPE targeting. This finding was supported by immunofluorescent staining of retinal cryosections, which were labeled with anti-RFP for tdTomato signaling and anti-visual arrestin to label photoreceptors, along with DAPI for nuclear staining. In comparison to the PBS group (FIG. 24A), both solid LNPs (FIG. 24B and FIG. 24C) displayed red tdTomato fluorescence, with 90.5% AC18:1-SS-K(R)R/8% PS solid LNP presenting more intense and extensive tdTomato signals. We also conducted RNP delivery using 50% AC18:1-SS-K(R)R/8% PS solid LNP. Compared to the PBS group (FIG. 23A), the solid LNP treatment (FIG. 23B) showed tdTomato signal, although the intensity was weaker than that observed with Cre mRNA. This is attributed to the Ai14 mouse model, which greatly underreports the editing efficiency of CRISPR genome editors such as Cas9 RNP or Cas9 mRNA/sgRNA. These results suggest that AC18:1-SS-K(R)R lipopeptide-based solid LNPs are reliable nanoplatforms for in vivo biomacromolecule delivery.

### Example 11—Lipopeptide-Based Solid LNPs Enable Delivery of NAD⁺

Fabrication of NAD⁺ loaded solid LNPs. The lipopeptide and helper lipids were mixed in methanol at the desired molar ratio to achieve a total lipid concentration of 20 mg/mL. NAD⁺ was dissolved in 10 mM citrate buffer at pH 5 to a concentration of 40 mg/mL. The lipids methanol solution and NAD⁺ aqueous solution were rapidly mixed at a 3:1 aqueous-to-methanol ratio by pipetting during vortexing, and the resulting solid LNP solution was dialyzed against 1×PBS for 2 hours. The solid LNP solution was then sonicated for 15 minutes before HPLC testing to assess loading efficiency.

Characterization of NAD⁺ loaded solid LNPs. Solid LNPs based on AC18:1-SS-K(R)R were studied for their capability for loading the small molecule NAD⁺ using 90% solid LNP and 98.5% solid LNP (see Table 10). The hydrodynamic sizes for both formulations were approximately 130 nm. The loading efficiency for both formulations was estimated to be around 20%. Additionally, the loading content for the 90% solid LNP and 98.5% Solid LNP formulations was found to be approximately 50% in each.

TABLE 10
Characterization of NAD⁺ loaded solid LNPs.

| | | | Loading | Loading |
|---|---|---|---|---|
| No. | Formulations | Size (nm) | efficiency (%) | content (%) |
| 1 | 90% solid LNP | 128.3 ± 3.9 | 20.7 ± 1.5 | 55.3 ± 1.7 |
| 2 | 98.5% solid LNP | 127.4 ± 2.2 | 19.0 ± 1.2 | 52.2 ± 3.1 |

### REFERENCES

(1) Chen, G.; Abdeen, A. A.; Wang, Y.; Shahi, P. K.; Robertson, S.; Xie, R.; Suzuki, M.; Pattnaik, B. R.; Saha, K.; Gong, S. A biodegradable nanocapsule delivers a Cas9 ribonucleoprotein complex for in vivo genome editing. Nat. Nanotechnol. 2019, 14 (10), 974-980.
(2) Lee, K.; Conboy, M.; Park, H. M.; Jiang, F.; Kim, H. J.; Dewitt, M. A.; Mackley, V. A.; Chang, K.; Rao, A.; Skinner, C. Nanoparticle delivery of Cas9 ribonucleoprotein and donor DNA in vivo induces homology-directed DNA repair. Nat. Biomed. Eng. 2017, 1 (11), 889-901.
(3) Mout, R.; Ray, M.; Yesilbag Tonga, G.; Lee, Y.-W.; Tay, T.; Sasaki, K.; Rotello, V. M. Direct cytosolic delivery of CRISPR/Cas9-ribonucleoprotein for efficient gene editing. ACS nano 2017, 11 (3), 2452-2458. Wang, Y.; Shahi, P. K.; Wang, X.; Xie, R.; Zhao, Y.; Wu, M.; Roge, S.; Pattnaik, B. R.; Gong, S. In vivo targeted delivery of nucleic acids and CRISPR genome editors enabled by GSH-responsive silica nanoparticles. J. Controlled Release 2021, 336, 296-309. Rui, Y.; Wilson, D. R.; Choi, J.; Varanasi, M.; Sanders, K.; Karlsson, J.; Lim, M.; Green, J. J. Carboxylated branched poly(beta-amino ester) nanoparticles enable robust cytosolic protein delivery and CRISPR-Cas9 gene editing. Sci Adv 2019, 5 (12), eaay3255. DOI: 10.1126/sciadv.aay3255.
(4) Staahl, B. T.; Benekareddy, M.; Coulon-Bainier, C.; Banfal, A. A.; Floor, S. N.; Sabo, J. K.; Urnes, C.; Munares, G. A.; Ghosh, A.; Doudna, J. A. Efficient genome editing in the mouse brain by local delivery of engineered Cas9 ribonucleoprotein complexes. Nat. Biotechnol. 2017, 35 (5), 431-434.
(5) Xie, R.; Wang, X.; Wang, Y.; Ye, M.; Zhao, Y.; Yandell, B. S.; Gong, S. pH-Responsive Polymer Nanoparticles for Efficient Delivery of Cas9 Ribonucleoprotein With or Without Donor DNA. Adv Mater 2022, 34 (23), 2110618. DOI: 10.1002/adma.202110618.
(6) Sharma, G.; Sharma, A. R.; Bhattacharya, M.; Lee, S. S.; Chakraborty, C. CRISPR-Cas9: A Preclinical and Clinical Perspective for the Treatment of Human Diseases. Mol. Ther. 2021, 29 (2), 571-586. DOI: 10.1016/j.ymthe.2020.09.028.
(7) Chandrasekaran, A. P.; Song, M.; Kim, K.-S.; Ramakrishna, S. Different methods of delivering CRISPR/Cas9 into cells. Prog. Mol. Biol. Transl. Sci. 2018, 159, 157-176. Lattanzi, A.; Meneghini, V.; Pavani, G.; Amor, F.; Ramadier, S.; Felix, T.; Antoniani, C.; Masson, C.; Alibeu, O.; Lee, C. Optimization of CRISPR/Cas9 delivery to human hematopoietic stem and progenitor cells for therapeutic genomic rearrangements. Mol. Ther. 2019, 27 (1), 137-150.
(8) D'Astolfo, D. S.; Pagliero, R. J.; Pras, A.; Karthaus, W. R.; Clevers, H.; Prasad, V.; Lebbink, R. J.; Rehmann, H.; Geijsen, N. Efficient intracellular delivery of native proteins. Cell 2015, 161 (3), 674-690.
(9) Fitch, C. A.; Platzer, G.; Okon, M.; Garcia-Moreno E, B.; McIntosh, L. P. Arginine: Its pKa value revisited. Protein Sci. 2015, 24 (5), 752-761.
(10) Wexselblatt, E.; Esko, J. D.; Tor, Y. On guanidinium and cellular uptake. J. Org. Chem. 2014, 79 (15), 6766-6774.
(11) Heyda, J.; Okur, H. I.; Hladilkovi, J.; Rembert, K. B.; Hunn, W.; Yang, T.; Dzubiella, J.; Jungwirth, P.; Cremer, P. S. Guanidinium can both cause and prevent the hydrophobic collapse of biomacromolecules. J. Am. Chem. Soc. 2017, 139 (2), 863-870.
(12) Pantos, A.; Tsogas, I.; Paleos, C. M. Guanidinium group: a versatile moiety inducing transport and multicompartmentalization in complementary membranes. Biochimica et Biophysica Acta (BBA)-Biomembranes 2008, 1778 (4), 811-823. Rothbard, J. B.; Jessop, T. C.; Wender, P. A. Adaptive translocation: the role of hydrogen bonding and membrane potential in the uptake of guanidinium-rich transporters into cells. Adv. Drug Del. Rev. 2005, 57 (4), 495-504.
(13) Rothbard, J. B.; Jessop, T. C.; Lewis, R. S.; Murray, B. A.; Wender, P. A. Role of membrane potential and hydrogen bonding in the mechanism of translocation of guanidinium-rich peptides into cells. J. Am. Chem. Soc. 2004, 126 (31), 9506-9507.
(14) Liu, C.; Wan, T.; Wang, H.; Zhang, S.; Ping, Y.; Cheng, Y. A boronic acid-rich dendrimer with robust and unprecedented efficiency for cytosolic protein delivery and CRISPR-Cas9 gene editing. Science advances 2019, 5 (6), eaaw8922.
(15) Barrios, A.; Estrada, M.; Moon, J. H. Carbamoylated Guanidine-Containing Polymers for Non-Covalent Functional Protein Delivery in Serum-Containing Media. Angew. Chem. 2022. Chang, H.; Lv, J.; Gao, X.; Wang, X.; Wang, H.; Chen, H.; He, X.; Li, L.; Cheng, Y. Rational design of a polymer with robust efficacy for intracellular protein and peptide delivery. Nano Lett. 2017, 17 (3), 1678-1684. Guo, S.; Huang, Q.; Wei, J.; Wang, S.; Wang, Y.; Wang, L.; Wang, R. Efficient intracellular delivery of native proteins facilitated by preorganized guanidiniums on pillar [5) arene skeleton. Nano Today 2022, 43, 101396. Lee, Y.-W.; Luther, D. C.; Goswami, R.; Jeon, T.; Clark, V.; Elia, J.; Gopalakrishnan, S.; Rotello, V. M. Direct cytosolic delivery of proteins through coengineering of proteins and polymeric delivery vehicles. J. Am. Chem. Soc. 2020, 142 (9), 4349-4355. Nair, J. B.; Mohapatra, S.; Ghosh, S.; Maiti, K. K. Novel lysosome targeted molecular transporter built on a guanidinium-poly-(propylene imine) hybrid dendron for efficient delivery of doxorubicin into cancer cells. Chem. Comm. 2015, 51 (12), 2403-2406. Yu, C.; Tan, E.; Xu, Y.; Lv, J.; Cheng, Y. A guanidinium-rich polymer for efficient cytosolic delivery of native proteins. Bioconj. Chem. 2018, 30 (2), 413-417. Yu, Z.; Zhang, Z.; Yan, J.; Zhao, Z.; Ge, C.; Song, Z.; Yin, L.; Tang, H. Guanidine-rich helical polypeptides bearing hydrophobic amino acid pendants for efficient gene delivery. Biomater. Sci. 2021, 9 (7), 2670-2678. Zhao, J.; Ullah, I.; Gao, B.; Guo, J.; Ren, X.-k.; Xia, S.; Zhang, W.; Feng, Y. Agmatine-grafted bioreducible poly (1-lysine) for gene delivery with low cytotoxicity and high efficiency. J. Mater. Chem. B 2020, 8 (12), 2418-2430. Xu, X.; Jiang, Q.; Zhang, X.; Nie, Y.; Zhang, Z.; Li, Y.; Cheng, G.; Gu, Z. Virus-inspired mimics: self-assembly of dendritic lipopeptides into arginine-rich nanovectors for improving gene delivery. J. Mater. Chem. B 2015, 3 (35), 7006-7010.
(16) Cheng, R.; Feng, F.; Meng, F.; Deng, C.; Feijen, J.; Zhong, Z. Glutathione-responsive nano-vehicles as a promising platform for targeted intracellular drug and gene delivery. J. Controlled Release 2011, 152 (1), 2-12.
(17) Mintzer, M. A.; Simanek, E. E. Nonviral vectors for gene delivery. Chem. Rev. 2009, 109 (2), 259-302. Semple, S. C.; Akinc, A.; Chen, J.; Sandhu, A. P.; Mui, B. L.; Cho, C. K.; Sah, D. W.; Stebbing, D.; Crosley, E. J.; Yaworski, E. Rational design of cationic lipids for siRNA delivery. Nat. Biotechnol. 2010, 28 (2), 172-176.
(18) Meng, F.; Cheng, R.; Deng, C.; Zhong, Z. Intracellular drug release nanosystems. Materials today 2012, 15 (10), 436-442.
(19) Madisen, L.; Zwingman, T. A.; Sunkin, S. M.; Oh, S. W.; Zariwala, H. A.; Gu, H.; Ng, L. L.; Palmiter, R. D.; Hawrylycz, M. J.; Jones, A. R. A robust and high-throughput Cre reporting and characterization system for the whole mouse brain. Nat. Neurosci. 2010, 13 (1), 133-140.
(20) Yiu, E. M.; Kornberg, A. J. Duchenne muscular dystrophy. J. Paediatr. Child Health 2015, 51 (8), 759-764.
(21) Sicinski, P.; Geng, Y.; Ryder-Cook, A. S.; Barnard, E. A.; Darlison, M. G.; Barnard, P. J. The molecular basis of muscular dystrophy in the mdx mouse: a point mutation. Science 1989, 244 (4912), 1578-1580.
(22) Happi Mbakam, C.; Lamothe, G.; Tremblay, G.; Tremblay, J. P. CRISPR-Cas9 Gene Therapy for Duchenne Muscular Dystrophy. Neurother. 2022, 1-11. Long, C.; McAnally, J. R.; Shelton, J. M.; Mireault, A. A.; Bassel-Duby, R.; Olson, E. N. Prevention of muscular dystrophy in mice by CRISPR/Cas9-mediated editing of germline DNA. Science 2014, 345 (6201), 1184-1188.
(23) Gustafson, H. H.; Holt-Casper, D.; Grainger, D. W.; Ghandehari, H. Nanoparticle uptake: the phagocyte problem. Nano today 2015, 10 (4), 487-510.
(24) Q. Xu, T. Zhang, Q. Wang, X. Jiang, A. Li, Y. Li, T. Huang, F. Li, Y. Hu, D. Ling, Uniformly sized iron oxide nanoparticles for efficient gene delivery to mesenchymal stem cells, International Journal of Pharmaceutics 552(1-2) (2018) 443-452.
(25) Y. Wang, P. K. Shahi, X. Wang, R. Xie, Y. Zhao, M. Wu, S. Roge, B. R. Pattnaik, S. Gong, In vivo targeted delivery of nucleic acids and CRISPR genome editors enabled by GSH-responsive silica nanoparticles, Journal of Controlled Release 336 (2021) 296-309.
(26) C. Fonseca, S. Simoes, R. Gaspar, Paclitaxel-loaded PLGA nanoparticles: preparation, physicochemical characterization and in vitro anti-tumoral activity, Journal of controlled release 83(2) (2002) 273-286.
(27) R. Harikrishnan, C. Balasundaram, M.-S. Heo, Poly d,l-lactide-co-glycolic acid (PLGA)-encapsulated vaccine on immune system in Epinephelus bruneus against Uronema marinum, Experimental parasitology 131(3) (2012) 325-332.
(28) Y. Liu, T. M. Reineke, Degradation of poly (glycoamidoamine) DNA delivery vehicles: polyamide hydrolysis at physiological conditions promotes DNA release, Biomacromolecules 11(2) (2010) 316-325.
(29) C. Zhang, R. Jin, P. Zhao, C. Lin, A family of cationic polyamides for in vitro and in vivo gene transfection, Acta biomaterialia 22 (2015) 120-130.
(30) B. W. Schlichtmann, B. Kalyanaraman, R. L. Schlichtmann, M. G. Panthani, V. Anantharam, A. G. Kanthasamy, S. K. Mallapragada, B. Narasimhan, Functionalized polyanhydride nanoparticles for improved treatment of mitochondrial dysfunction, Journal of Biomedical Materials Research Part B: Applied Biomaterials 110(2) (2022) 450-459.
(31) F. N. Al-Heibshy, E. Başaran, R. Arslan, N. Öztürk, İ. Vural, M. Demirel, Preparation, characterization and pharmacokinetic evaluation of rosuvastatin calcium incorporated cyclodextrin-polyanhydride nanoparticles, Drug Development and Industrial Pharmacy 45(10) (2019) 1635-1645.
(32) S. K. Alsaiari, S. Patil, M. Alyami, K. O. Alamoudi, F. A. Aleisa, J. S. Merzaban, M. Li, N. M. Khashab, Endosomal escape and delivery of CRISPR/Cas9 genome editing machinery enabled by nanoscale zeolitic imidazolate framework, Journal of the American Chemical Society 140(1) (2018) 143-146.
(33) Zhu, M. et al. Guanidinium-Rich Lipopeptide-Based Nanoparticle Enables Efficient Gene Editing in Skeletal Muscles. ACS Appl Mater Interfaces 15, 10464-10476, doi:10.1021/acsami.2c21683 (2023).
(34) Maeki, M., Uno, S., Niwa, A., Okada, Y. & Tokeshi, M. Microfluidic technologies and devices for lipid nanoparticle-based RNA delivery. J Control Release 344, 80-96, doi:10.1016/j.jconrel.2022.02.017 (2022).
(35) Yang, S., Zhou, J. & Li, D. Functions and Diseases of the Retinal Pigment Epithelium. Front Pharmacol 12, 727870, doi:10.3389/fphar.2021.727870 (2021).

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Likewise, the use of the terms “comprising,” “including,” “containing,” etc. shall be understood to disclose embodiments using the terms “consisting essentially of” and “consisting of” The phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Illustrative Features

The present technology may include, but is not limited to, the features and combinations of features recited in the following numbered paragraphs, it being understood that the following paragraphs should not be interpreted as limiting the scope of the claims as appended hereto or mandating that all such features must necessarily be included in such claims:

P1. A compound of Formula I,

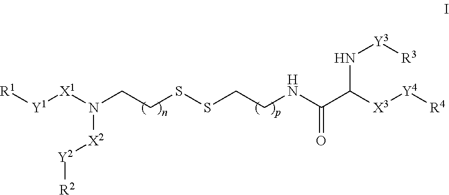

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently absent, C(O)O, or C(O)NH;
Y³ is absent, C(O), C(O)O, or C(O)NH;
Y⁴ is C(O)O, C(O)NH, NHC(O)O, or NHC(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid, peptide, or isopeptide; and further provided that if one of R³ and R⁴ is absent, the other is present; and
n and p are each independently 1, 2, 3, 4, or 5.

P2. A compound of paragraph P1 having Formula IA,

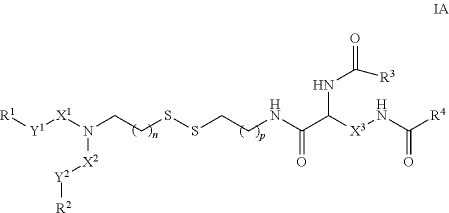

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
n and p are each independently 1, 2, 3, 4, or 5.

P3. The compound of paragraph P1 or paragraph P2, wherein the compound of Formula I is a compound of Formula II,

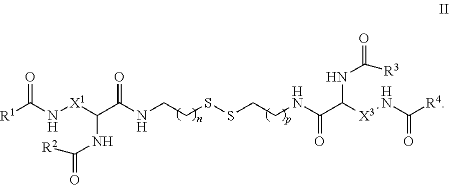

P4. The compound of paragraph P3, wherein the compound of Formula II is a compound of Formula IIA,

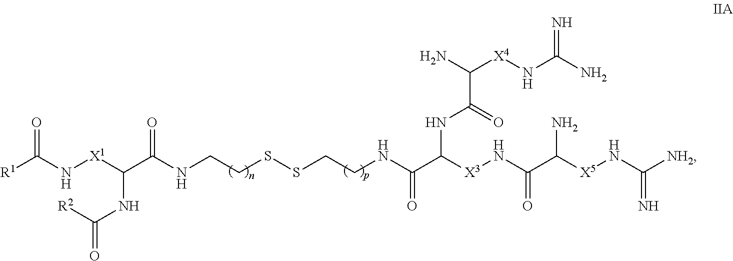

wherein
X⁴ and X⁵ are independently selected from C₁₋₆ alkylene or C₂₋₆ alkenylene groups.

P5. The compound of paragraph P1 or paragraph P2, wherein the compound of Formula I is a compound of Formula III,

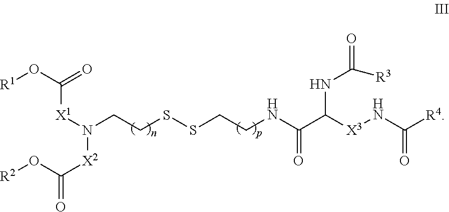

P6. The compound of any one of paragraphs P1-P5, wherein X¹ and X² are independently selected from unsubstituted C₁₋₄ alkylene or C₂₋₄ alkenylene.

P7. The compound of any one of paragraphs P1-P6, wherein X¹ and X² are each butylene.

P8. The compound of any one of paragraphs P1-P7, wherein R¹ and R² are independently selected from unsubstituted C₁₂₋₂₄ alkyl or C₁₂₋₂₄ alkenyl groups.

P9. The compound of any one of paragraphs P1-P8, wherein R¹ and R² are independently selected from unsubstituted C₁₆₋₂₀ alkyl or C₁₆₋₂₀ alkenyl groups.

P10. The compound of any one of paragraphs P1-P9, wherein R¹ and R² are independently a C₁₈ alkyl group or a C₁₈ alkenyl group having 1 or 2 carbon-carbon double bonds.

P11. The compound of any one of paragraphs P1-P10, wherein R³ and R⁴ are independently selected from C₂₋₄ alkyl groups substituted with 1, 2, or 3 ionizable functional groups.

P12. The compound of any one of paragraphs P1-P11, wherein R³ and R⁴ are substituted with 1 or 2 ionizable functional groups.

P13. The compound of any one of paragraphs P1-P12, wherein each ionizable functional group is independently selected from NH₂, NHR, NR₂, guanidine, imidazole, or amidine, wherein each R is independently an unsubstituted C₁₋₆ alkyl, phenyl, or benzyl group.

P14. The compound of paragraph P13, wherein at least one ionizable functional group is guanidine.

P15. The compound of any one of paragraphs P1-P14, wherein R³ and R⁴ are each substituted with an NH₂ group and a guanidine group.

P16. The compound of any one of paragraphs P1-P15 selected from the group consisting of

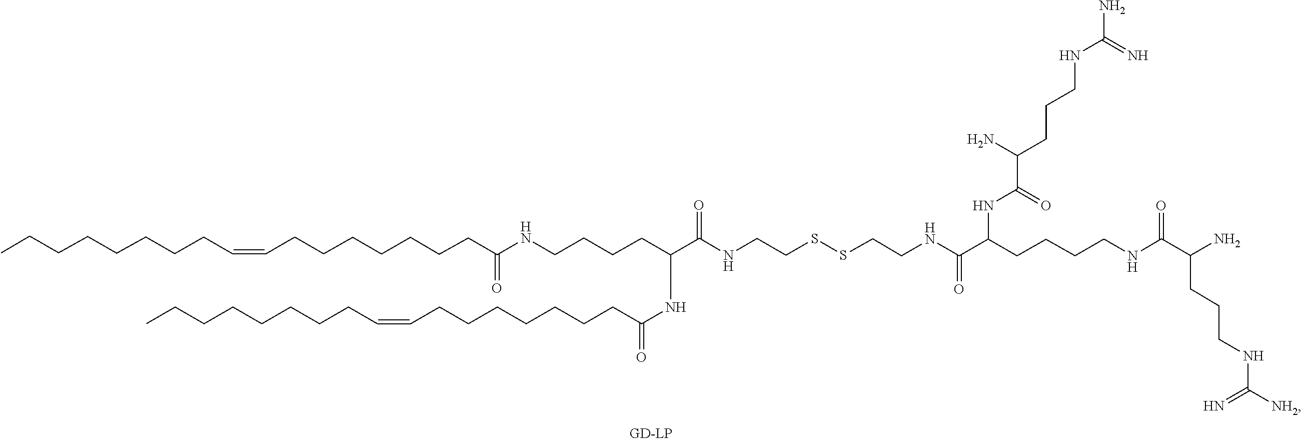 [SMILES: CCCCCCCC/C=C\CCCCCCCC(=O)NCCCCC(NC(=O)CCCCCCC/C=C\CCCCCCCC)C(=O)NCCSSCCNC(=O)C(CCCCNC(=O)C(N)CCCNC(=N)N)NC(=O)C(N)CCCNC(=N)N]

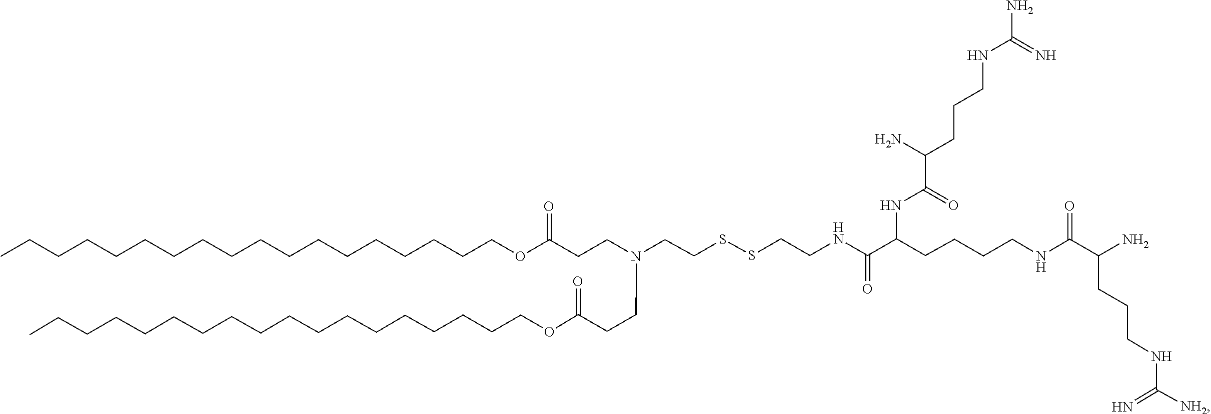 [SMILES: CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC]

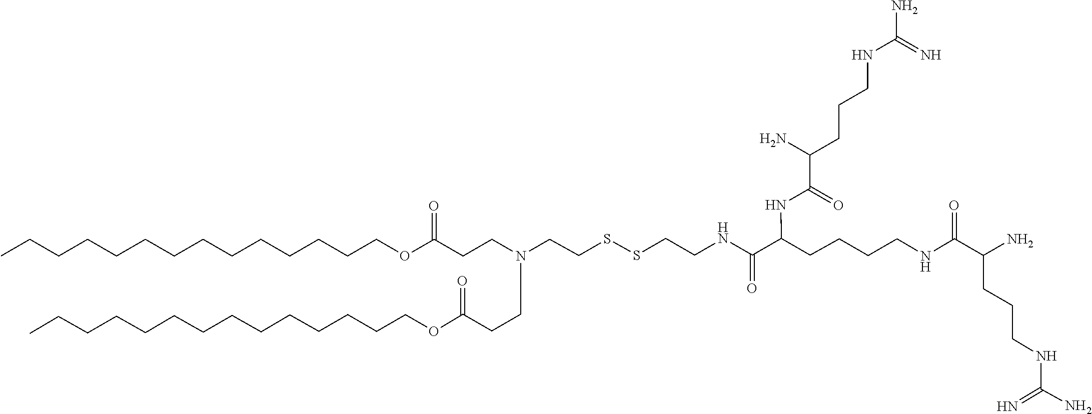 [SMILES: CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC]

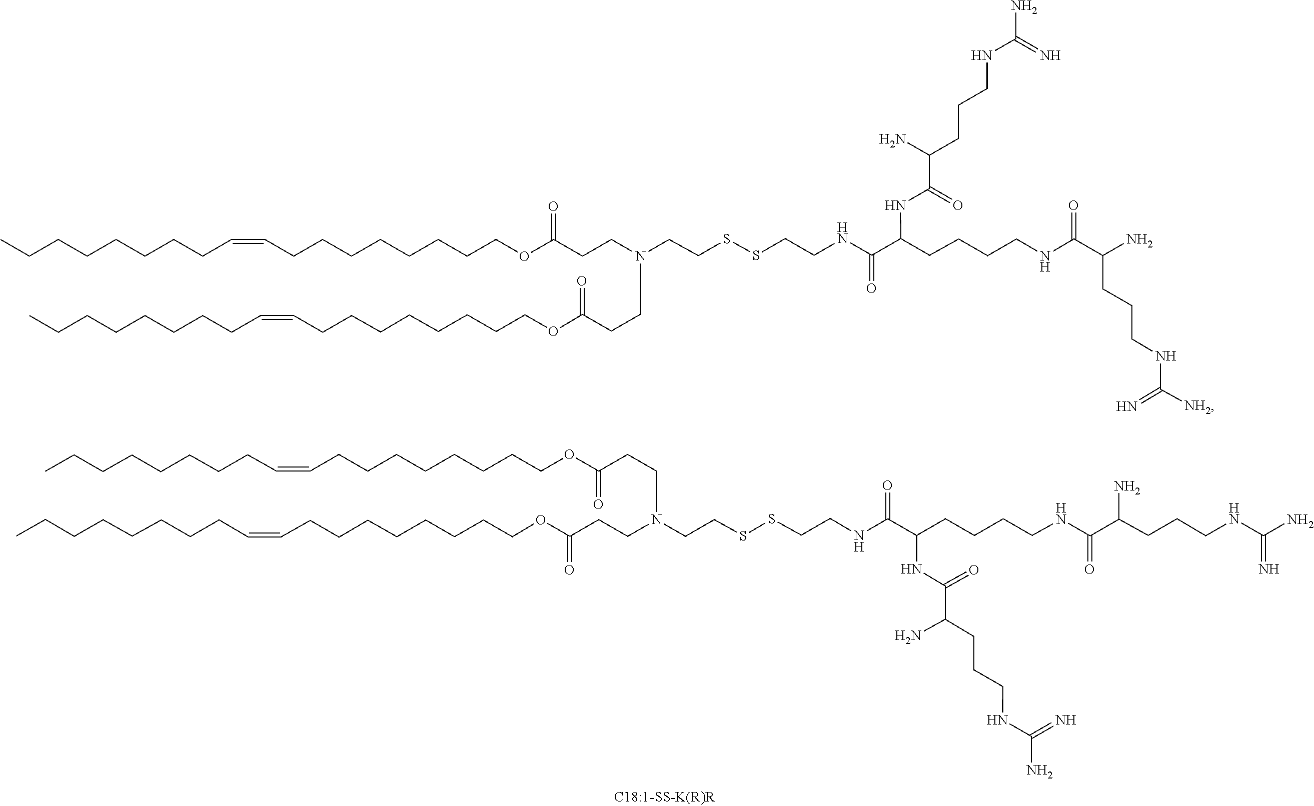 [SMILES: CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC ; CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC]

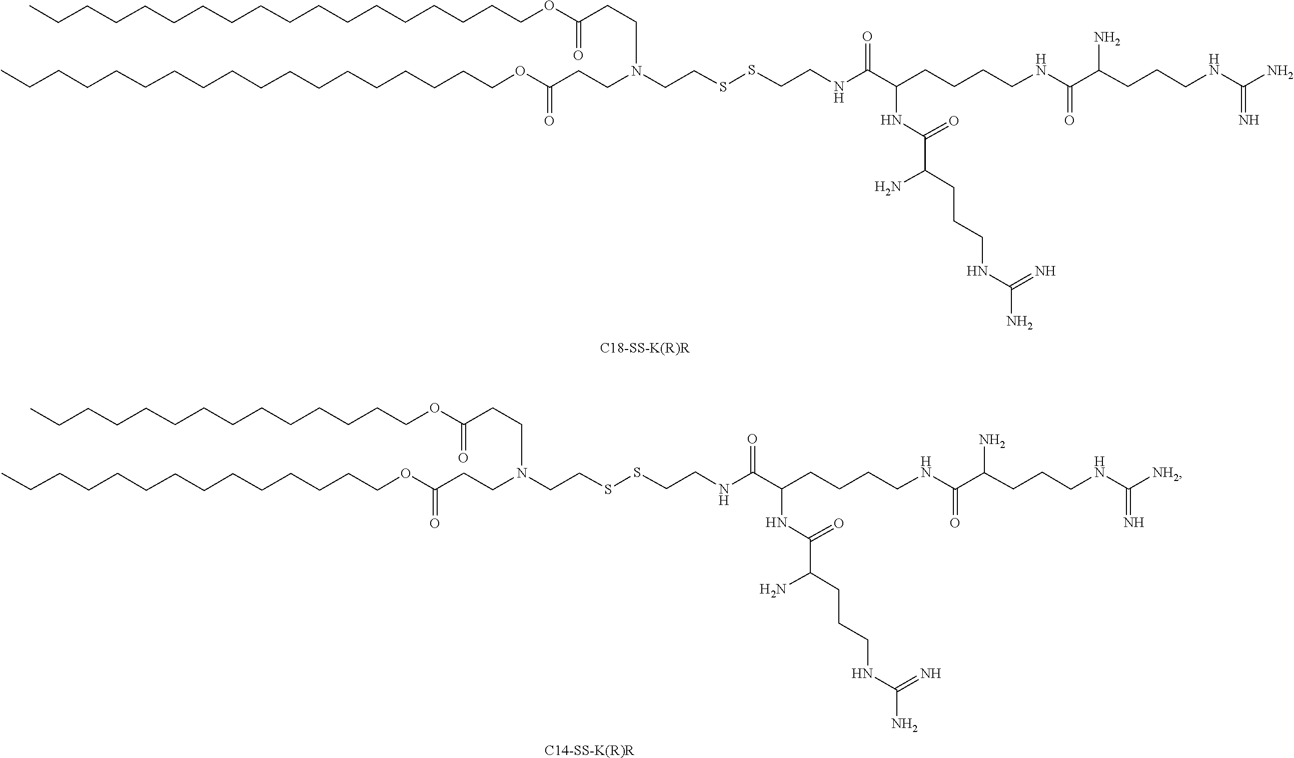 [SMILES: CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC ; CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC ; "C18-SS-K(R)R" ; "C14-SS-K(R)R"]

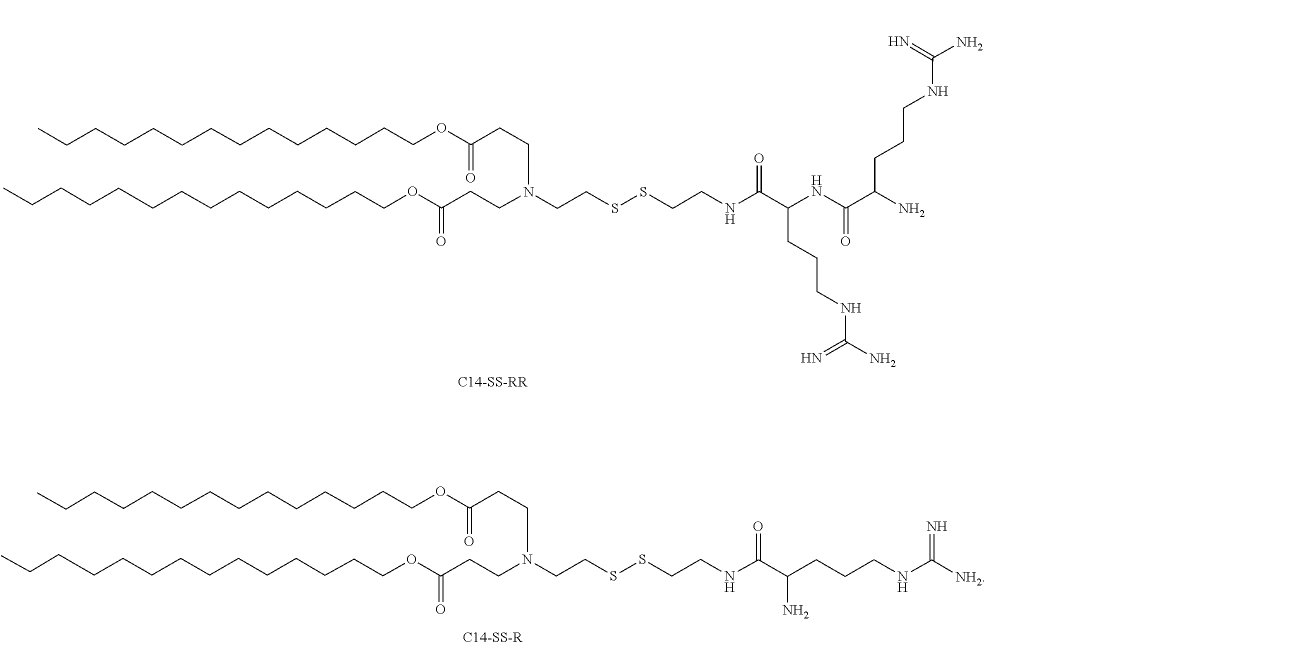 [SMILES: CCCCCCCCCCCCCCCOC(=O)CCN(CCSSCCNC(=O)C(N)CCCNC(=N)N)CCC(=O)OCCCCCCCCCCCCCC ; CCCCCCCCCCCCCCCOC(=O)CCN(CCSSCCNC(=O)C(CCCNC(=N)N)NC(=O)C(N)CCCNC(=N)N)CCC(=O)OCCCCCCCCCCCCCC ; "C14-SS-RR"]

P17. A compound of Formula IV,

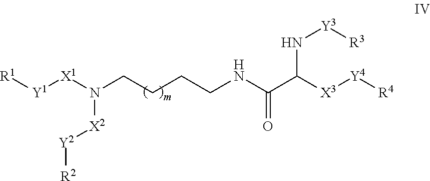

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently absent, C(O)O, or C(O)NH;
Y³ is absent, C(O), C(O)O, or C(O)NH;
Y⁴ is C(O)O, or C(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid, peptide, or isopeptide; and further provided that if one of R³ and R⁴ is absent, the other is present; and
m is 1, 2, 3, 4, 5, 6, 7, 8, 9, 10 or 11.

P18. The compound of P17, of Formula IVA,

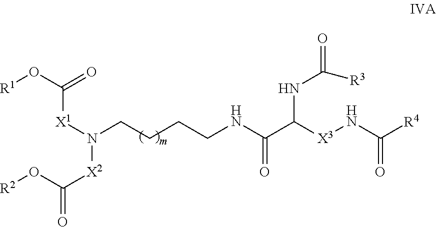

a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
m is 1, 2, 3, 4, 5, 6, 7, 8, 9, 10 or 11.

P19. A compound of any one of paragraphs P17 or P18, of Formula IVB,

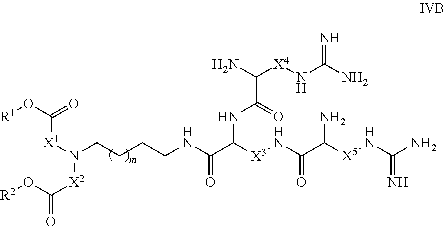

wherein
X⁴ and X⁵ are independently selected from C₁₋₆ alkylene or C₂₋₆ alkenylene groups and the remaining variables may have any of the values disclosed herein, including but not limited to the those disclosed for Formula IV above.

P20. The compound of any one of paragraphs P17-P19, wherein X¹ and X² are independently selected from unsubstituted C₁₋₄ alkylene or C₂₋₄ alkenylene.

P21. The compound of any one of paragraphs P17-P20, wherein X¹ and X² are each butylene.

P22 The compound of any one of paragraphs P17-P21, wherein R¹ and R² are independently selected from unsubstituted C₁₂₋₂₄ alkyl or C₁₂₋₂₄ alkenyl groups.

P23. The compound of any one of paragraphs P17-P22, wherein R¹ and R² are independently selected from unsubstituted C₁₆₋₂₀ alkyl or C₁₆₋₂₀ alkenyl groups.

P24. The compound of any one of paragraphs P17-P23, wherein R¹ and R² are independently a C₁₈ alkyl group or a C₁₈ alkenyl group having 1 or 2 carbon-carbon double bonds.

P25. The compound of any one of paragraphs P17-P24, wherein R³ and R⁴ are independently selected from C₂₋₄ alkyl groups substituted with 1, 2, or 3 ionizable functional groups.

P26. The compound of any one of paragraphs P17-P25, wherein R³ and R⁴ are substituted with 1 or 2 ionizable functional groups.

P27. The compound of any one of paragraphs P17-P26, wherein each ionizable functional group is independently selected from NH₂, NHR, NR₂, guanidine, imidazole, or amidine, wherein each R is independently an unsubstituted C₁₋₆ alkyl, phenyl, or benzyl group.

P28. The compound of paragraph P27, wherein at least one ionizable functional group is guanidine.

P29. The compound of any one of paragraphs P17-P28, wherein R³ and R⁴ are each substituted with an NH₂ group and a guanidine group.

P30. The compound of any one of paragraphs P17 to P29, wherein X⁴ and X⁵ are independently selected from C₁₋₆ alkylene.

P31. The compound of any one of paragraphs P17 to P30 selected from the group consisting of:

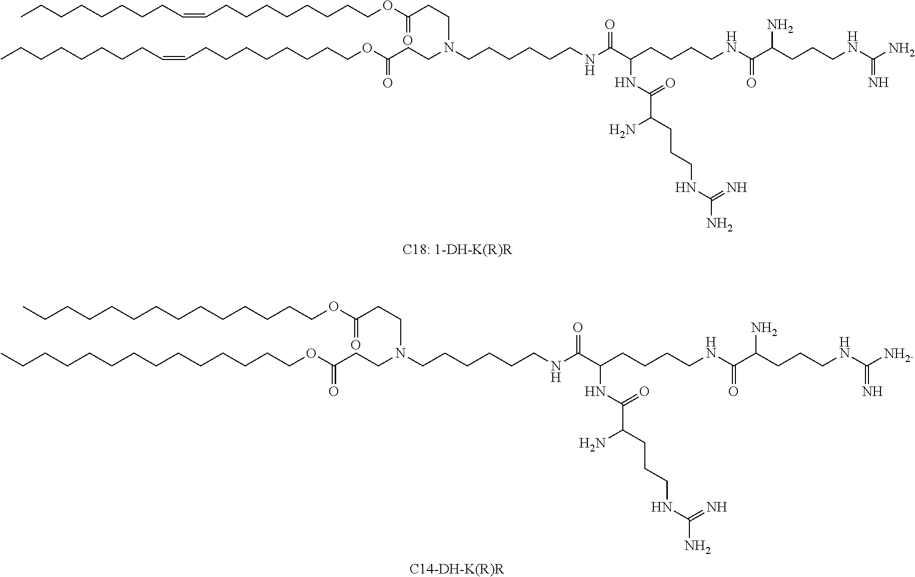 [SMILES: CCCCCCCC/C=C\CCCCCCCCOC(=O)CCN(CCCCCCNC(=O)C(CCCCNC(=O)C(N)CCCNC(=N)N)NC(=O)C(N)CCCNC(=N)N)CCC(=O)OCCCCCCCC/C=C\CCCCCCCC ; CCCCCCCCCCCCCCOC(=O)CCN(CCCCCCNC(=O)C(CCCCNC(=O)C(N)CCCNC(=N)N)NC(=O)C(N)CCCNC(=N)N)CCC(=O)OCCCCCCCCCCCCCC ; "C14-DH-K(R)R"]

P32. A nanoparticle comprising a compound of of any one of paragraphs P1-P31.

P33. The nanoparticle of paragraph P32 further comprising a PEG-lipid.

P34. The nanoparticle of paragraph P33, wherein the PEG-lipid is PEG-DSPE and/or PEG-DMG.

P35. The nanoparticle of any one of paragraphs P32-P34 further comprising a structural lipid.

P36. The nanoparticle of paragraph P35, wherein the structural lipid is cholesterol and/or 3-sitosterol.

P37. The nanoparticle of any one of paragraphs P32-P36 further comprising a phospholipid.

P38. The nanoparticle of paragraph P37, wherein the phospholipid is DSPC, DOPS and/or DOPE.

P39. The nanoparticle of any one of paragraphs P32-P38, wherein the compound of Formula I comprises at least 50 mol % of the nanoparticle.

P40. The nanoparticle of paragraph P39, wherein the compound of Formula I comprises at least 80 mol % of the nanoparticle.

P41. The nanoparticle of paragraph P39, wherein the compound of Formula I comprises 90 to 99 mol % of the nanoparticle.

P42. The nanoparticle of any one of paragraphs P32-P41 further comprising a payload selected from DNA, RNA, a polypeptide, a ribonucleoprotein complex (RNP), or any combination of two or more thereof.

P43. The nanoparticle of paragraph P42, wherein the payload is selected from the group consisting of RNP, plasmid DNA (pDNA), single-stranded donor oligonucleotide (ssODN), antisense oligonucleotides (ASO) complementary DNA (cDNA), NAD⁺, NADH, messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof.

P44. The nanoparticle of paragraph P42 or paragraph P43, wherein the payload is a Cas9 RNP, Cas12 RNP, Cas13 RNP, Cas14 RNP, CasΦ RNP, each optionally in combination with an ssODN, or the payload is a plasmid or mRNA for a Cas polypeptide and an sgRNA.

P45. The nanoparticle of any one of paragraphs P42-P44, wherein the payload is Cas9 mRNA, a plasmid encoding Cas9, Cas12 mRNA, a plasmid encoding Cas12, Cas13 mRNA, a plasmid encoding Cas13, Cas14 mRNA, a plasmid encoding Cas14, CasΦ mRNA, or a plasmid encoding CasΦ, each optionally in combination with an sgRNA, or the payload is a combination of any two or more thereof.

P46. The nanoparticle of any one of paragraphs P32-P45, having an average hydrodynamic diameter of 50 nm to 500 nm.

P47. The nanoparticle of any one of paragraphs P32-P46, having a zeta potential of about −40 mV to about +40 mV.

P48. The nanoparticle of any one of paragraphs P32-P47, wherein the compound coats an exterior surface of an inorganic nanoparticle.

P49. The nanoparticle of paragraph P48, wherein the inorganic nanoparticle is a silica nanoparticle.

P50. The nanoparticle of paragraph P49, wherein the silica nanoparticle comprises crosslinked polysiloxanes comprising siloxy subunits having one or more of the structures of Formulas IV, IVA, or IVB:

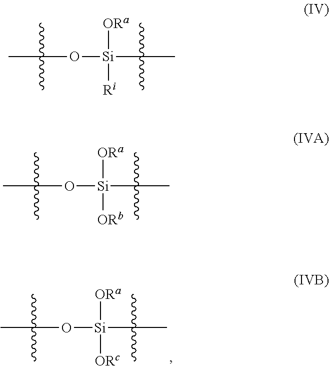

wherein
Rⁱ at each occurrence is independently selected from the group consisting of C₁₋₁₂ alkyl and C₂₋₁₂ alkenyl groups, optionally substituted with a substituent selected from the group consisting of halogen and NR¹², wherein each occurrence of R¹ is independently selected from H or a C₁₋₁₂ alkyl group, or two R¹ groups, together with the N atom to which they are attached, form a pyrrolidine or piperidine ring;
R^{a} and R^{b} at each occurrence in the polysiloxane are independently selected from a bond to a Si of another polysiloxane chain or C₁₋₆ alkyl groups, and
R^{c} is selected from C₂₋₆ alkenyl groups.

P51. The nanoparticle of paragraph P50, wherein the crosslinks between polysiloxanes comprise disulfide linkages.

P52. The nanoparticle of any one of paragraphs P49-P51, wherein the exterior surface of the silica nanoparticle comprises a silica network.

P53. The nanoparticle of paragraph P52, wherein the exterior surface of the silica nanoparticle comprises surface-modifying groups attached to and surrounding the silica network, wherein the surface-modifying groups comprise polyethylene glycol (PEG), polysarcosine, polyzwitterion, polycation, polyanion, or combinations of two or more thereof.

P54. The nanoparticles of any one of paragraphs P50-P53, wherein the polysiloxanes further comprise silyloxy subunits having structure (IVE):

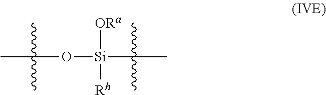

wherein
R^{a} at each occurrence is independently selected from a bond to a Si of another polysiloxane chain or a C₁₋₁₂ alkyl group; and
R^{h} at each occurrence is a moiety comprising a weakly basic group.

P55. The nanoparticle of paragraph P54, wherein the weakly basic group is selected from imidazolyl, pyridinyl, tetrahydroquinolinyl, or indolinyl groups, or a combination any two or more thereof.

P56. The nanoparticle of paragraph P54 or paragraph P55, wherein R^{h} has the structure —(CH₂)ₙ-L-Z, and wherein
L is a bond or is a linking group selected from —C(O)NH—, —O—, —NH—, —C(O)—, or —C(O)O;
Z is at each occurrence is independently a picolinyl, lutidinyl, indolinyl, tetrahydroquinolinyl, quinolinyl, imidazolyl, or pyridinyl group; and
n is 0, 1, 2, 3, or 4.

P57. The nanoparticle of any one of paragraphs P54-P56, wherein R^{h} has the structure (D):

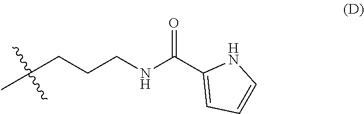 [SMILES: *CCCNC(=O)c1ccc[nH]1]

P58. The nanoparticle of any one of paragraphs P49-P57, wherein the crosslinked polysiloxanes comprise crosslinking subunits having the structure (V):

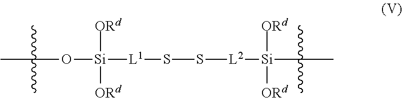

wherein:
L¹ and L² at each occurrence are independently selected from a C₁₋₆ alkylene group; and
R^{d} at each occurrence is independently selected from a bond to another polysiloxane chain or a C₁₋₆ alkyl group.

P59. The nanoparticle of paragraph P58, wherein R^{d} at each occurrence is ethyl.

P60. The nanoparticle of paragraph P58 or paragraph P59, wherein each of L¹ and L² is propylene at each occurrence.

P61. The nanoparticle of any one of paragraphs P50-P60, wherein the polysiloxanes comprise a plurality of siloxy subunits having the structure (VI):

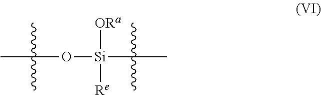

wherein:
R^{a} at each occurrence is selected from a bond to Si from another polysiloxane chain or a C₁₋₆ alkyl group, and
R^{e} at each occurrence is the surface-modifying group, optionally including a C₁₋₆ linker group connecting the surface-modifying group to the Si atom to which R^{e} is attached.

P62. The nanoparticle of paragraph P61, wherein the C₁₋₆ linker group is present and connected to the surface-modifying group directly or via an amine, ether, amide, ester, urethane, urea, imine, or sulfide group.

P63. The nanoparticle of paragraph P61 or paragraph P62, wherein the C₁₋₆ linker group is present and is —NHC(O)NH—(C₂₋₅ alkylene)-, —NHC(O)—(C₂₋₅ alkylene)-, —C(O)NH—(C₂₋₅ alkylene)-, —NH—(C₂₋₅ alkylene)-, —O—(C₂₋₅ alkylene)-, —S—(C₂₋₅ alkylene)-, —OC(O)NH—(C₂₋₅ alkylene)-, or —NHC(O)O—(C₂₋₅ alkylene)-.

P64. The nanoparticle of any one of paragraphs P53-P63, wherein the surface-modifying groups are PEG or polysarcosine.

P65. The nanoparticle of paragraph P64, wherein the surface-modifying groups comprise PEG attached to a siloxy subunit having the structure (VII)

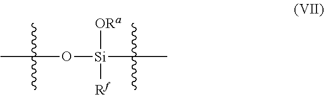

wherein R^{a} at each occurrence is selected from a bond to Si from another polysiloxane chain or a C₁₋₆ alkyl group, and R^{f} has the structure (E1):

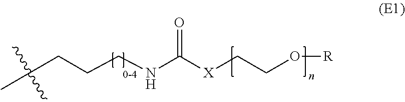

wherein X is O, NH, or CH₂O, and R is a C₁₋₆ alkyl, targeting ligand, a cell-penetrating peptide (CPP), or imaging agent.

P66. The nanoparticle of any one of paragraphs P53-P65, wherein the surface-modifying group is a polyzwitterion selected from poly(carboxybetaine methacrylate) (PCBMA), poly(sulfobetaine methacrylate) (PSBMA), poly(2-methacryloyloxyethyl phosphorylcholine) (PMPC), or combinations of two or more thereof; or
the surface-modifying group is a cationic polymer selected from polyethyleneimine (PEI), polylysine, polyarginine, polyamidoamine (PAMAM), or combinations of two or more thereof; or
the surface-modifying group is an anionic polymer selected from poly(glutamic acid) or poly(acrylic acid).

P67. A method of delivering a payload into a mammalian cell, the method comprising exposing the cell to a nanoparticle of any one of paragraphs P42-P68.

P68. The method of paragraph P67, wherein the mammalian cell is selected from a kidney cell, fibroblast cell, monocyte/macrophage cell, or melanoma cell.

P69. A method of treating a condition or disorder in a subject that may be ameliorated by a payload selected from RNA, DNA or RNP, the method comprising administering to the subject an effective amount of a nanoparticle of any one of paragraphs P42-P68.

Other embodiments are set forth in the following claims, along with the full scope of equivalents to which such claims are entitled.

1. A compound of Formula I, 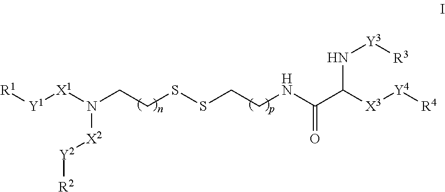
a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently absent, C(O)O, or C(O)NH;
Y³ is absent, C(O), C(O)O, or C(O)NH;
Y⁴ is C(O)O, C(O)NH, NHC(O)O, or NHC(O)NH;
R¹ and R² are independently selected from unsubstituted C₈₋₂₄ alkyl or C₈₋₂₄ alkenyl groups;
R³ and R⁴ are independently absent or selected from an amino acid residue, a peptide or isopeptide comprising 2-10 amino acid residues, or a C₁₋₁₂ alkyl group, each of which is optionally substituted with 1, 2, or 3 ionizable functional groups such that at least one ionizable functional group is present on at least one of R³ and R⁴, provided that Y³ is absent when R³ is an amino acid, peptide, or isopeptide, and Y⁴ is absent when R⁴ is an amino acid, peptide, or isopeptide; and further provided that if one of R³ and R⁴ is absent, the other is present; and
n and p are each independently 1, 2, 3, 4, or 5.

2. A compound of Formula IA, 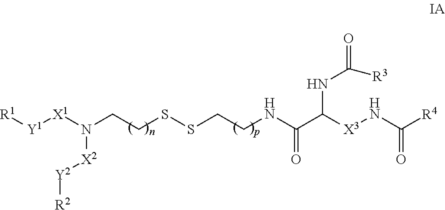
a stereoisomer thereof, and/or a pharmaceutically acceptable salt thereof,
wherein
X¹ and X² are independently absent or selected from unsubstituted C₁₋₆ alkylene or C₂₋₆ alkenylene;
X³ is selected from unsubstituted C₁₋₆ alkylene or C₂₋₆ alkenylene;
Y¹ and Y² are each independently selected from C(O)O or C(O)NH;
R¹ and R² are independently selected from unsubstituted C₈₋₂₄ alkyl or C₈₋₂₄ alkenyl groups;
R³ and R⁴ are independently selected from C₁₋₁₀ alkyl groups substituted with 1, 2, or 3 ionizable functional groups; and
n and p are each independently 1, 2, 3, 4, or 5.

3. The compound of claim 1, wherein the compound of Formula I is a compound of Formula II,

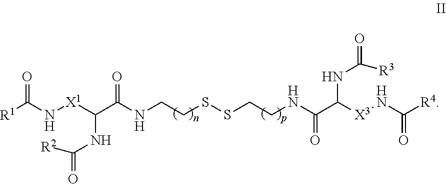

4. The compound of claim 3, wherein the compound of Formula II is a compound of Formula IIA, 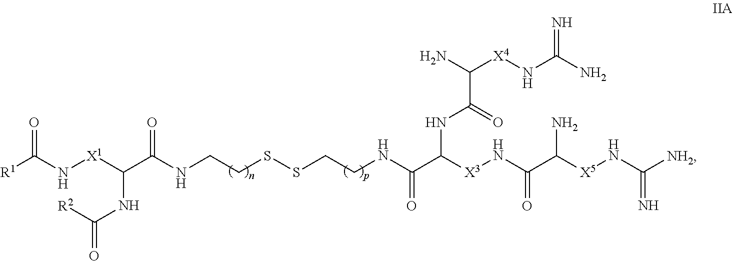
wherein
X⁴ and X⁵ are independently selected from C₁₋₆ alkylene or C₂₋₆ alkenylene groups.

5. The compound of claim 1, wherein the compound of Formula I is a compound of Formula III,

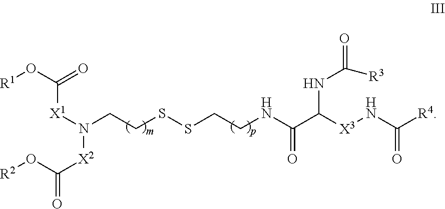

6. The compound of claim 1, wherein X¹ and X² are independently selected from unsubstituted C₁₋₄ alkylene or C₂₋₄ alkenylene.

7. The compound of claim 1, wherein X¹ and X² are each butylene.

8. The compound of claim 1, wherein R¹ and R² are independently selected from unsubstituted C₁₂₋₂₄ alkyl or C₁₂₋₂₄ alkenyl groups.

9. The compound of claim 1, wherein R¹ and R² are independently selected from unsubstituted C₁₆₋₂₀ alkyl or C₁₆₋₂₀ alkenyl groups.

10. The compound of claim 1, wherein R¹ and R² are independently a C₁₈ alkyl group or a C₁₈ alkenyl group having 1 or 2 carbon-carbon double bonds.

11. The compound of claim 1, wherein R³ and R⁴ are independently selected from C₂₋₄ alkyl groups substituted with 1, 2, or 3 ionizable functional groups.

12. The compound of claim 1, wherein R³ and R⁴ are substituted with 1 or 2 ionizable functional groups.

13. The compound of claim 1, wherein each ionizable functional group is independently selected from NH₂, NHR, NR₂, guanidine, imidazole, or amidine, wherein each R is independently an unsubstituted C₁₋₆ alkyl, phenyl, or benzyl group.

14. The compound of claim 13, wherein at least one ionizable functional group is guanidine.

15. The compound of claim 1, wherein R³ and R⁴ are each substituted with an NH₂ group and a guanidine group.

16. The compound of claim 1 selected from the group consisting of

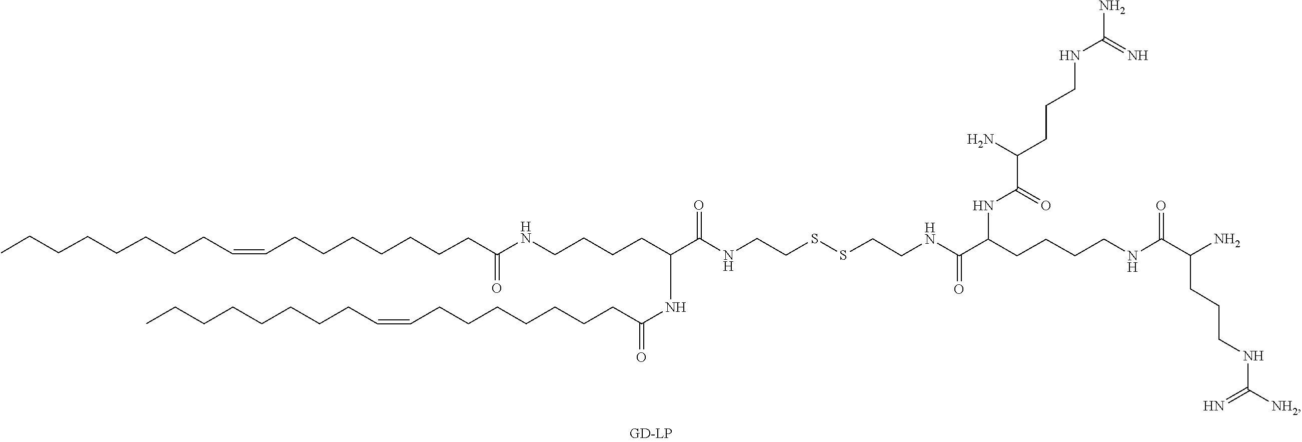 [SMILES: CCCCCCCC/C=C\CCCCCCCC(=O)NCCCCC(NC(=O)CCCCCCC/C=C\CCCCCCCC)C(=O)NCCSSCCNC(=O)C(CCCCNC(=O)C(N)CCCNC(=N)N)NC(=O)C(N)CCCNC(=N)N]

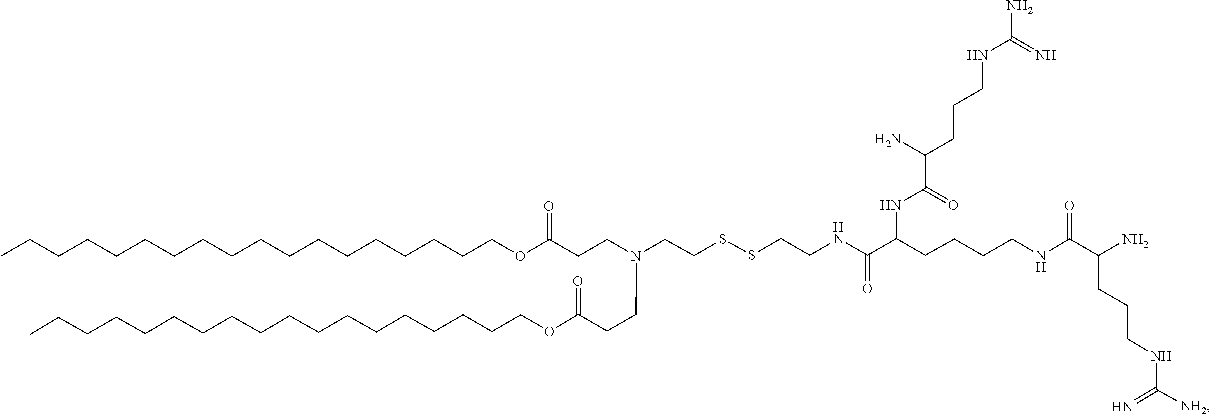 [SMILES: CCCCCCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCCCCCC]

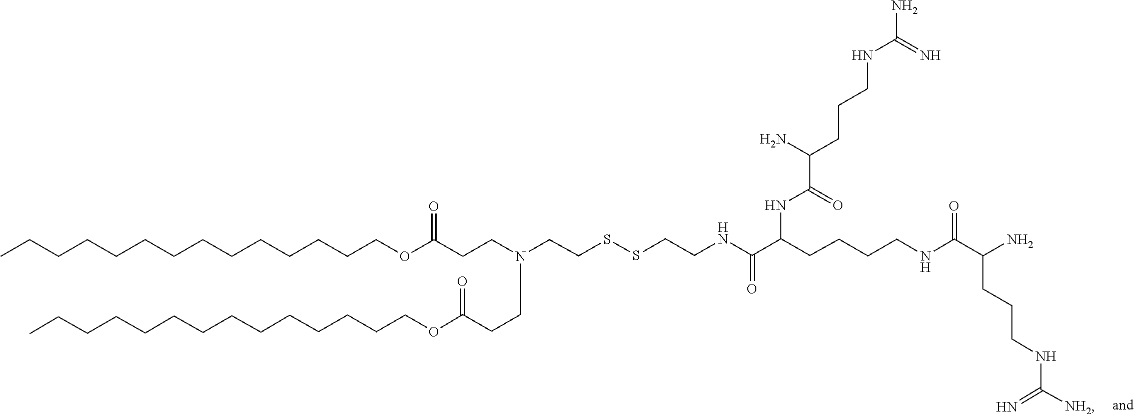 [SMILES: CCCCCCCCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCCCCCCCC]

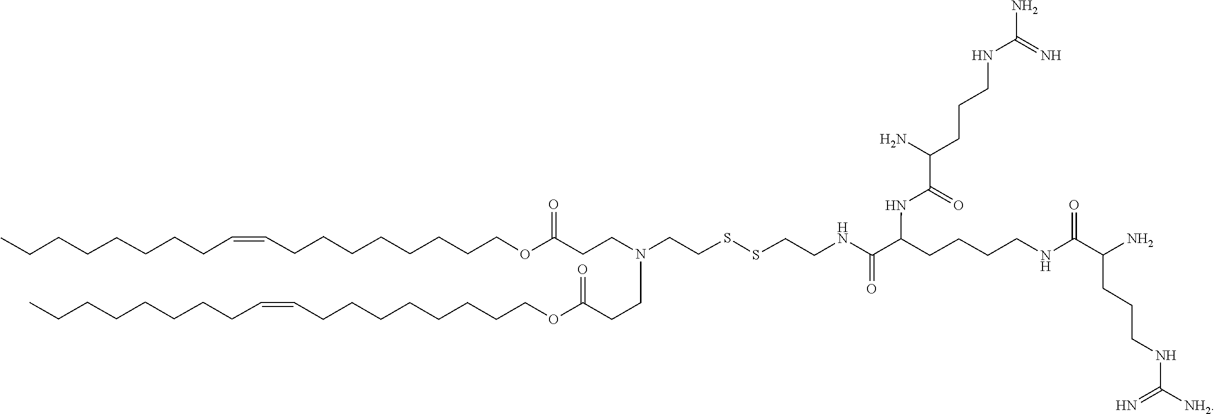 [SMILES: CCCCCCCC/C=C\CCCCCCCCOC(=O)CCN(CCSSCCNC(=O)C(CCCCNC(=O)C(N)CCCNC(=N)N)NC(=O)C(N)CCCNC(=N)N)CCC(=O)OCCCCCCCC/C=C\CCCCCCCC]

17. A nanoparticle comprising a compound of Formula I of claim 1.

18.-26. (canceled)

27. The nanoparticle of claim 1 further comprising a payload selected from DNA, RNA, a polypeptide, a ribonucleoprotein complex (RNP), or any combination of two or more thereof.

28.-51. (canceled)

52. A method of delivering a payload into a mammalian cell, the method comprising exposing the cell to a nanoparticle of claim 27.

53. (canceled)

54. A method of treating a condition or disorder in a subject that may be ameliorated by a payload selected from RNA, DNA or RNP, the method comprising administering to the subject an effective amount of a nanoparticle of claim 27.
